(19) United States
(12) Patent Application Publication
(10) Pub. No.: US 2022/0117993 A1
(43) Pub. Date: Apr. 21, 2022

(54) Compounds Useful in HIV Therapy

(71) Applicants: GlaxoSmithKline Intellectual Property (No. 2) Limited, Brentford, Middlesex (GB); VIIV Healthcare Company, Wilmington, DE (US)

(72) Inventors: John Franklin MILLER, Research Triangle Park, NC (US); David TEMELKOFF, Research Triangle Park, NC (US); Emile Johann VELTHUISEN, Research Triangle Park, NC (US); Martha Alicia DE LA ROSA, Research Triangle Park, NC (US); Lita SUWANDI, Research Triangle Park, NC (US); B. Narasimhulu NAIDU, Branford, CT (US)

(73) Assignees: GlaxoSmithKline Intellectual Property (No. 2) Limited, Brentford, Middlesex (GB); VIIV Healthcare Company, Wilmington, DE (US)

(21) Appl. No.: 17/266,675

(22) PCT Filed: Aug. 8, 2019
(86) PCT No.: PCT/IB2019/056761

§ 371 Date: Feb. 8, 2021

Related U.S. Application Data
(60) Provisional application No. 62/716,494, filed on Aug. 9, 2018.

(51) Int. Cl. A61K 31/7076; C07H 19/16; A61K 45/06; A61P 31/18

(52) U.S. Cl. CPC A61K 31/7076; A61P 31/18; A61K 45/06; C07H 19/16

(57) ABSTRACT

The invention relates to compounds of the Formula:

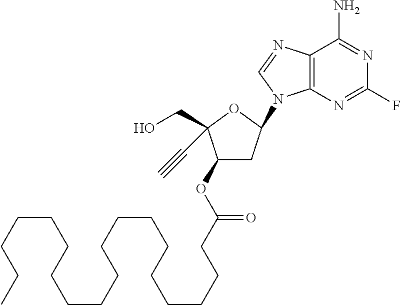 [SMILES: C#C[C@]1(CO)O[C@@H](n2cnc3c(N)nc(F)nc32)C[C@H]1OC(=O)CCCCCCCCCCCCCCCCCCC]

salts thereof, pharmaceutical compositions thereof, combinations thereof, as well as therapeutic methods of treatment and prevention.

### CROSS-REFERENCE TO RELATED APPLICATIONS

The present application claims priority to U.S. Provisional Application Ser. No. 62/716,494 filed Aug. 9, 2018, the disclosure of which is incorporated by reference herein in its entirety.

### FIELD OF THE INVENTION

The present invention relates to compounds, pharmaceutical compositions, and methods of use thereof in connection with individuals infected with HIV.

### BACKGROUND OF THE INVENTION

Human immunodeficiency virus type 1 (HIV-1) infection leads to the contraction of acquired immune deficiency disease (AIDS). The number of cases of HIV continues to rise, and currently an estimated over thirty-five million individuals worldwide suffer from HIV infection e.g., http://www.sciencedirect.com/science/article/pii/S235230181630087X?via %3Dihub

Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still believed to be required due to a number of issues including, but not limited to undesirable drug-drug interactions; drug-food interactions; non-adherence to therapy; drug resistance due to mutation of the enzyme target; and inflammation related to the immunologic damage caused by the HIV infection.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur and the survival and quality of life are not normalized as compared to uninfected persons [Lohse Ann Intern Med 2007 146; 87-95], Indeed, the incidence of several non-AIDS morbidities and mortalities, such as cardiovascular disease, frailty, and neurocognitive impairment, are increased in HAART-suppressed, HIV-infected subjects [Deeks Annu Rev Med 2011; 62:141-155], This increased incidence of non-AIDS morbidity/mortality occurs in the context of, and is potentially caused by, elevated systemic inflammation related to the immunologic damage caused by HIV infection [Hunt J Infect Dis 2014] [Byakagwa J Infect Dis 2014][Tenorio J Infect Dis 2014].

Modern antiretroviral therapy (ART) has the ability to effectively suppress HIV replication and improve health outcomes for HIV-infected persons, but is believed to not be capable of completely eliminating HIV viral reservoirs within the individual. HIV genomes can remain latent within mostly immune cells in the infected individual and may reactivate at anytime, such that after interruption of ART, virus replication typically resumes within weeks. In a handful of individuals, the size of this viral reservoir has been significantly reduced and upon cessation of ART, the rebound of viral replication has been delayed [Henrich T J J Infect Dis 2013] [Henrich T J Ann Intern Med 2014], In one case, the viral reservoir was eliminated during treatment of leukemia and no viral rebound was observed during several years of follow-up [Hutter G N Engl J Med 2009], These examples suggest the concept that reduction or elimination of the viral reservoir may be possible and can lead to viral remission or cure. As such, ways have been pursued to eliminate the viral reservoir, by direct molecular means, including excision of viral genomes with CRISPR/Cas9 systems, or to induce reactivation of the latent reservoir during ART so that the latent cells are eliminated. Induction of the latent reservoir typically results in either direct death of the latently infected cell or killing of the induced cell by the immune system after the virus is made visible. As this is performed during ART, viral genomes produced are believed to not result in the infection of new cells and the size of the reservoir may decay.

HAART therapies are often complex because a combination of different drugs must be administered often daily to the patient to avoid the rapid emergence of drug-resistant HIV-1 variants. Despite the positive impact of HAART on patient survival, drug resistance can still occur.

Current guidelines recommend that therapy includes three fully active drugs. See e.g. https://aidsinfo.nih.gov/guidelines Typically, first-line therapies combine two to three drugs targeting the viral enzymes reverse transcriptase and integrase. It is believed that sustained successful treatment of HIV-1-infected patients with antiretroviral drugs employ the continued development of new and improved drugs that are effective against HIV strains that have formed resistance to approved drugs. For example an individual on a regimen containing 3TC/FTC may select for the M184V mutation that reduces susceptibility to these drugs by >100 fold. See eg., https://hivdb.stanford.edu/dr-summary/resistance-notes/NRTI

Another way to potentially address preventing formation of mutations is to increase patient adherence to a drug regimen. One manner that may accomplish this is by reducing the dosing frequency. For parenteral administration, it is believed to be advantageous to have drug substances with high lipophilicity in order to reduce solubility and limit the release rate within interstitial fluid. However, most nucleoside reverse transcriptase inhibitors are hydrophilic thereby potentially limiting their use as long acting parenteral agents.

There remains a need for compounds which may the shortcomings set forth above.

### SUMMARY OF THE INVENTION

In one aspect, there is provide a compound of the formula (I):

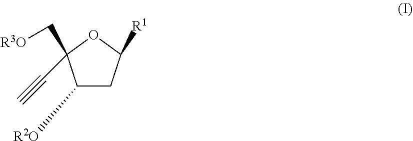

wherein:
R¹ is:

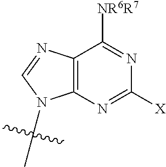

wherein:

X is selected from the group consisting of NH₂, F and Cl

R² is —C(═O)—R⁴ wherein R⁴ is selected from the group consisting of (C₁-C₂₅) alkyl, (C₂-C₂₅) alkenyl, (C₂-C₂₅) alkynyl, and (C₁-C₁₀) haloalkyl; wherein each of R⁴ may be optionally substituted by (C₁-C₆) alkyl, Cl, F, oxo, or (C₁-C₆) alkoxy;

R³ is selected from the group consisting of H and —(C═O)—O—R⁵ wherein R⁵ is selected from the group consisting of (C₁-C₁₀) alkyl, (C₂-C₁₀) alkenyl and (C₂-C₁₀) alkynyl; and

R⁶ and R⁷ are independently selected from the group consisting of H— and —C(═O)—OR⁸ wherein R⁸ is (C₁-C₁₀) alkyl;

or a pharmaceutically acceptable salt thereof.

In another aspect, there is provided a compound of the formula (II):

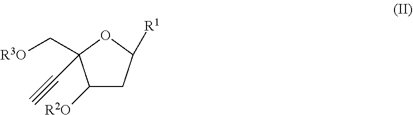

### R¹ is:

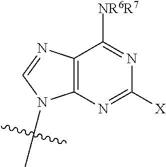

wherein:

X is selected from the group consisting of NH₂, F and Cl

R² is —C(═O)—R⁴ wherein R⁴ is selected from the group consisting of (C₁-C₂₅) alkyl, (C₂-C₂₅) alkenyl, (C₂-C₂₅) alkynyl, and (C₁-C₁₀) haloalkyl; wherein each of R⁴ may be optionally substituted by (C₁-C₆) alkyl, Cl, F, oxo, or (C₁-C₆) alkoxy;

R³ is selected from the group consisting of H and —(C═O)—O—R⁵ wherein R⁵ is selected from the group consisting of (C₁-C₁₀) alkyl, (C₂-C₁₀) alkenyl and (C₂-C₁₀) alkynyl; and

R⁶ and R⁷ are independently selected from the group consisting of H— and —C(═O)—OR⁸ wherein R⁸ is (C₁-C₁₀) alkyl;

or a pharmaceutically acceptable salt thereof.

In another aspect, the invention provides pharmaceutical compositions comprising a compound of Formulae (I)-(II) or a pharmaceutically acceptable salt thereof and an excipient

In another aspect, the invention provides a method of treating or preventing an HIV infection in a subject at risk for developing an HIV infection, comprising administering to the subject a compound of Formulae (I)-(II), or a pharmaceutically acceptable salt thereof.

In another aspect, there is provided a compound of Formulae (I)-(II) or a pharmaceutically acceptable salt thereof for use in therapy.

In another aspect, there is provided a compound of Formulae (I)-(II) or a pharmaceutically acceptable salt thereof for use in treating or preventing an HIV infection.

In another aspect, there is provided the use of a compound of Formulae (I)-(II) or a pharmaceutically acceptable salt thereof in the manufacture of a medicament for treating or preventing an HIV infection.

These and other aspects are encompassed by the invention as set forth herein.

### BRIEF DESCRIPTION OF THE DRAWINGS

FIG. 1 represents the mean concentration-time profiles for Example 6 and EFdA after single subcutaneous injection of the compound of Example 6 at 20 mg/kg in Wistar Han rats (N=3/time point).

FIG. 2 represents the mean concentration-time profiles for Example 6 and EFdA after single intramuscular injection of the compound of Example 6 at 20 mg/kg in Wistar Han rats (N=3/time point).

FIG. 3 represents the mean concentration-time profiles for Example 21 and EFdA after single intramuscular injection of the compound of Example 21 at 20 mg/kg in Wistar Han rats (N=3/time point).

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

Where used herein the terms such as “a compound of formula (I), (Ia), (II) and (IIa)” and “compounds of formulae (I), (Ia), (II) and (IIa)” are intended to refer to each and all of the compounds defined herein, i.e., the compounds of formulae (I), (Ia), (II) and (IIa).

As used herein, and unless otherwise specified, the following definitions are applicable:

“Alkyl” refers to a monovalent saturated aliphatic hydrocarbon group having from, e.g., 1 to 25 carbon, e.g., 1 to 10 carbon atoms atoms and, in some embodiments, from 1 to 6 carbon atoms. “(Cₓ-C_{y}) alkyl” refers to alkyl groups having from x to y carbon atoms. The term “alkyl” includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term alkyl may also be interpreted to encompass alkylene groups as defined below.

“Alkylene” refers to divalent saturated aliphatic hydrocarbon groups that may having e.g., from 1 to 25 carbon atoms. The alkylene groups include branched and straight chain hydrocarbyl groups. For example, “(C₁-C₆)alkylene” is meant to include methylene, ethylene, propylene, 2-methypropylene, dimethylethylene, pentylene, and so forth. As such, the term “propylene” could be exemplified by the following structure:

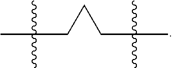 [SMILES: *CCC*]

Likewise, the term “dimethylbutylene” could be exemplified by any of the following three structures or more:

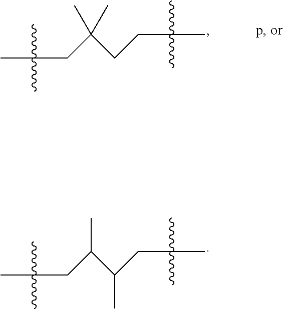

Furthermore, the term “(C₁-C₆)alkylene” is meant to include such branched chain hydrocarbyl groups as cyclopropylmethylene, which could be exemplified by the following structure:

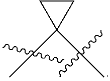 [SMILES: *C1(*)CC1]

“Alkenyl” refers to a linear or branched hydrocarbon group having, e.g., from 2 to 25, e.g., 2 to 20, e.g., 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, isopropylene, 1,3-butadienyl, and the like Polyalkenyl substituents are also encompassed by this definition.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₂₅), (C₂-C₂₀), or (C₂-C₆)alkynyl is meant to include ethynyl, propynyl, and the like. Polyalkynyl substituents are also encompassed by this definition.

“Alkoxy” refers to the group —O-alkyl wherein alkyl is defined herein, e.g., C₁ to C₆ alkoxy. Alkoxy includes, by way of example, methoxy, ethoxy, n-propoxy, isopropoxy, n-butoxy, t-butoxy, sec-butoxy, and n-pentoxy.

“AUC” refers to the area under the plot of plasma concentration of drug (not logarithm of the concentration) against time after drug administration.

“EC₅₀” refers to the concentration of a drug that gives half-maximal response.

“IC₅₀” refers to the half-maximal inhibitory concentration of a drug. Sometimes, it is also converted to the pIC₅₀ scale (−log IC₅₀), in which higher values indicate exponentially greater potency.

“Haloalkyl” refers to substitution of an alkyl group with 1 to 3 halo groups (e.g., bifluoromethyl or trifluoromethyl).

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

The term “heteroatom” means nitrogen, oxygen, or sulfur and includes any oxidized form of nitrogen, such as N(O) {N⁺—O⁻} and sulfur such as S(O) and S(O)₂, and the quaternized form of any basic nitrogen.

“Oxo” refers to a (═O) group.

“Polymorphism” refers to when two or more clearly different phenotypes exist in the same population of a species where the occurrence of more than one form or morph. In order to be classified as such, morphs must occupy the same habitat at the same time and belong to a panmictic population (one with random mating).

“Racemates” refers to a mixture of enantiomers. In an embodiment of the invention, the compounds of Formulae I, Ia, II, or IIa, or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

The term ‘atropisomer’ refers to a stereoisomer resulting from an axis of asymmetry. This can result from restricted rotation about a single bond where the rotational barrier is high enough to allow differentiation of the isomeric species up to and including complete isolation of stable non-interconverting diastereomer or enantiomeric species. One skilled in the art will recognize that upon installing a nonsymmetrical R^{x} to core, the formation of atropisomers is possible. In addition, once a second chiral center is installed in a given molecule containing an atropisomer, the two chiral elements taken together can create diastereomeric and enantiomeric stereochemical species. Depending upon the substitution about the Cx axis, interconversion between the atropisomers may or may not be possible and may depend on temperature. In some instances, the atropisomers may interconvert rapidly at room temperature and not resolve under ambient conditions. Other situations may allow for resolution and isolation but interconversion can occur over a period of seconds to hours or even days or months such that optical purity is degraded measurably overtime. Yet other species may be completely restricted from interconversion under ambient and/or elevated temperatures such that resolution and isolation is possible and yields stable species. When known, the resolved atropisomers were named using the helical nomenclature. For this designation, only the two ligands of highest priority in front and behind the axis are considered. When the turn priority from the front ligand 1 to the rear ligand 1 is clockwise, the configuration is P, if counterclockwise it is M.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” or “subject” refers to mammals and includes humans and non-human mammals.

Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Where specific compounds or generic formulae are drawn that have aromatic rings, such as aryl or heteroaryl rings, then it will be understood by one of still in the art that the particular aromatic location of any double bonds are a blend of equivalent positions even if they are drawn in different locations from compound to compound or from formula to formula. For example, in the two pyridine rings (A and B) below, the double bonds are drawn in different locations, however, they are known to be the same structure and compound:

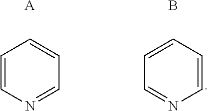

The present invention includes compounds as well as their pharmaceutically acceptable salts. Accordingly, the word “or” in the context of “a compound or a pharmaceutically acceptable salt thereof” is understood to refer to either: 1) a compound alone or a compound and a pharmaceutically acceptable salt thereof (alternative), or 2) a compound and a pharmaceutically acceptable salt thereof (in combination).

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—. In a term such as “—C(R^{x})₂”, it should be understood that the two R^{x} groups can be the same, or they can be different if R^{x} is defined as having more than one possible identity. In addition, certain substituents are drawn as —R^{x}R^{y}, where the indicates a bond adjacent to the parent molecule and R^{y} being the terminal portion of the functionality. Similarly, it is understood that the above definitions are not intended to include impermissible substitution patterns (e.g., methyl substituted with 5 fluoro groups). Such impermissible substitution patterns are well known to the skilled artisan.

In one aspect, there is provide a compound of the formula (I):

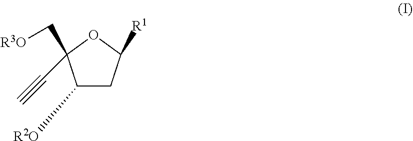

wherein:

R¹ is:

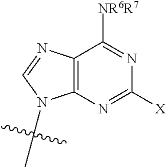

X is selected from the group consisting of NH₂, F and Cl

R² is —C(═O)—R⁴ wherein R⁴ is selected from the group consisting of (C₁-C₂₅) alkyl, (C₂-C₂₅) alkenyl, (C₂-C₂₅) alkynyl, and (C₁-C₁₀) haloalkyl; wherein each of R⁴ may be optionally substituted by (C₁-C₆) alkyl, Cl, F, oxo, or (C₁-C₆) alkoxy;

R³ is selected from the group consisting of H and —(C═O)—O—R⁵ wherein R⁵ is selected from the group consisting of (C₁-C₁₀) alkyl, (C₂-C₁₀) alkenyl and (C₂-C₁₀) alkynyl; and

R⁶ and R⁷ are independently selected from the group consisting of H— and —C(═O)—OR⁸ wherein R⁸ is (C₁-C₁₀) alkyl;

or a pharmaceutically acceptable salt thereof.

In another aspect, there is provide a compound of the formula (Ia):

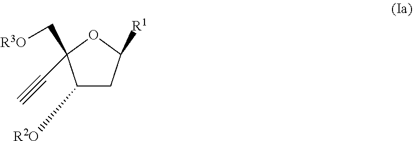

wherein:

R¹ is:

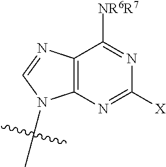

X is selected from the group consisting of NH₂, F and Cl

R² is —C(═O)—R⁴ wherein R⁴ is selected from the group consisting of (C₁-C₂₅) alkyl, (C₆)cycloalkyl and —(C₆)aryl; wherein each of (C₁-C₂₅) alkyl may be optionally substituted by (C₁-C₆) alkyl, Cl, F, oxo, (C₁-C₆) alkoxy; (C₆)cycloalkyl or —(C₆)aryl, wherein each (C₆)cycloalkyl may or be optionally substituted by (C₁-C₂₅) alkyl, and each (C₆)aryl may be optionally substituted by —O—(C═O)—(C₁-C₆ alkyl) or (C₁-C₆)alkyl;

R³ is selected from the group consisting of H and —(C═O)—O—R⁵ wherein R⁵ is selected from the group consisting of (C₁-C₁₀) alkyl, (C₂-C₁₀) alkenyl and (C₂-C₁₀) alkynyl; and

R⁶ and R⁷ are independently selected from the group consisting of H— and —C(═O)—R⁸ wherein R⁸ is (C₁-C₁₅) alkyl;

or a pharmaceutically acceptable salt thereof.

In another aspect, there is provided a compound of the formula (II):

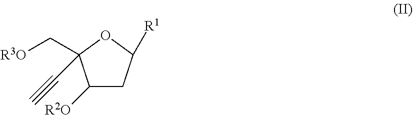

### R¹ is:

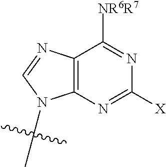

wherein:

X is selected from the group consisting of NH₂, F and Cl

R² is —C(═O)—R⁴ wherein R⁴ is selected from the group consisting of (C₁-C₂₅) alkyl, (C₂-C₂₅) alkenyl, (C₂-C₂₅) alkynyl, and (C₁-C₁₀) haloalkyl; wherein each of R⁴ may be optionally substituted by (C₁-C₆) alkyl, Cl, F, oxo, or (C₁-C₆) alkoxy;

R³ is selected from the group consisting of H and —(C═O)—O—R⁵ wherein R⁵ is selected from the group consisting of (C₁-C₁₀) alkyl, (C₂-C₁₀) alkenyl and (C₂-C₁₀) alkynyl; and

R⁶ and R⁷ are independently selected from the group consisting of H— and —C(═O)—OR⁸ wherein R⁸ is (C₁-C₁₀) alkyl;

or a pharmaceutically acceptable salt thereof.

In another aspect, there is provided a compound of the formula (IIa):

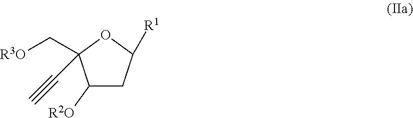

### R¹ is:

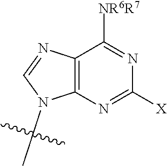

X is selected from the group consisting of NH₂, F and Cl

R² is —C(═O)—R⁴ wherein R⁴ is selected from the group consisting of (C₁-C₂₅) alkyl, (C₆)cycloalkyl and —(C₆)aryl; wherein each of (C₁-C₂₅) alkyl may be optionally substituted by (C₁-C₆) alkyl, Cl, F, oxo, (C₁-C₆) alkoxy; (C₆)cycloalkyl or (C₆)aryl, wherein each (C₆)cycloalkyl may or be optionally substituted by (C₁-C₂₅) alkyl, and each (C₆)aryl may be optionally substituted by —O—(C═O)—(C₁-C₆ alkyl) or (C₁-C₆)alkyl;

R³ is selected from the group consisting of H and —(C═O)—O—R⁵ wherein R⁵ is selected from the group consisting of (C₁-C₁₀) alkyl, (C₂-C₁₀) alkenyl and (C₂-C₁₀) alkynyl; and

R⁶ and R⁷ are independently selected from the group consisting of H— and —C(═O)—R⁸ wherein R⁸ is (C₁-C₁₅) alkyl;

or a pharmaceutically acceptable salt thereof.

Preferably, in embodiments of the formulae (I)-(II), R⁴ is selected from the group consisting of (C₁-C₂₅) alkyl, (C₂-C₂₅) alkenyl and (C₂-C₂₅) alkynyl.

Preferably, in embodiments of the formulae (I)-(II), R⁴ is selected from (C₁-C₂₅) alkyl.

Preferably, in embodiments of the formulae (I)-(II), R³ is H.

Preferably in embodiments of the formulae (I)-(II), R³ is —(C═O)—O—R⁵

Preferably in embodiments of the formulae (I)-(II), R⁵ is (C₁-C₁₀) alkyl.

Preferably in embodiments of the formulae (I)-(II), R⁵ is C₂ alkyl.

Preferably in embodiments of the formulae (I)-(II), X is F.

Preferably in embodiments of the formulae (Ia) and (IIa), R⁴—(C₆)cycloalkyl, In various embodiments, —C₆(cycloalkyl) may be optionally substituted by one or more of (C₁-C₆) alkyl, Cl, F, oxo, or (C₁-C₆) alkoxy. Preferred substitutents are (C₅)alkyl and —C(CH₃)₃.

Preferably in embodiments of the formulae (Ia) and (IIa), R4 is —(CH₂)ₑ—(C₆)aryl wherein e is 0 or an integer ranging from 1 to 6. In various embodiments, e is 1. In various embodiments, e is 4. In various embodiments, —(C₆)aryl may be optionally substituted by one or more of —O—(C═O)—(C₁-C₄ alkyl) or (C₁-C₆)alkyl. Preferred substituents include (CH₃) and —O—(C═O)—(C₁alkyl).

In another aspect of the present invention, the invention may encompass various individual compounds. As an example, such specific compounds may be selected from the group consisting of (Table 1):

TABLE 1

| Example | Structure | Chemical Name |
|---|---|---|
| 1 | 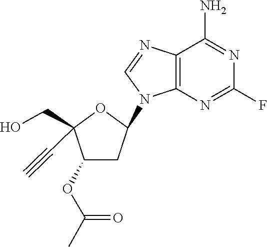 [SMILES: C#C[C@]1(CO)O[C@@H](n2cnc3c(N)nc(F)nc32)C[C@@H]1OC(C)=O] | (2R,3S,5R)-5-(6-amino-2-fluoro-9H- purin-9-yl)-2-ethynyl-2- (hydroxymethyl)tetrahydrofuran-3-yl acetate |
| 2 | 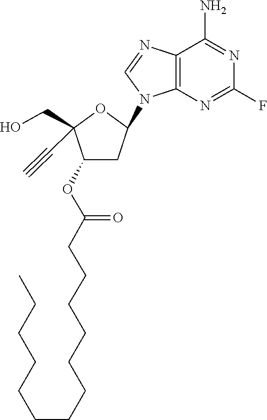 [SMILES: C#C[C@]1(CO)O[C@@H](n2cnc3c(N)nc(F)nc32)C[C@@H]1OC(=O)CCCCCCCCCCCCC] | (2R,3S,5R)-5-(6-amino-2-fluoro-9H- purin-9-yl)-2-ethynyl-2- (hydroxymethyl)tetrahydrofuran-3-yl tetradecanoate |
| 3 | 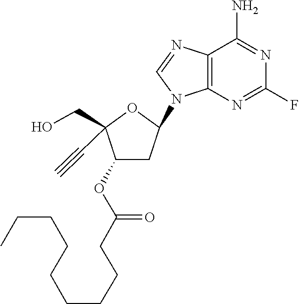 [SMILES: C#C[C@]1(CO)O[C@@H](n2cnc3c(N)nc(F)nc32)C[C@@H]1OC(=O)CCCCCCCCC] | (2R,3S,5R)-5-(6-amino-2-fluoro-9H- purin-9-yl)-2-ethynyl-2- (hydroxymethyl)tetrahydrofuran-3-yl decanoate |
| 4 | 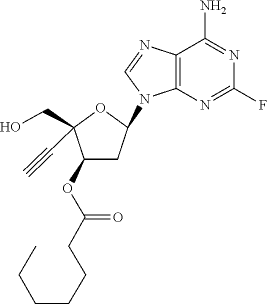 [SMILES: C#C[C@]1(CO)O[C@@H](n2cnc3c(N)nc(F)nc32)C[C@H]1OC(=O)CCCCCC] | (2R,3S,5R)-5-(6-amino-2-fluoro-9H- purin-9-yl)-2-ethynyl-2- (hydroxymethyl)tetrahydrofuran-3-yl heptanoate |
| 5 | 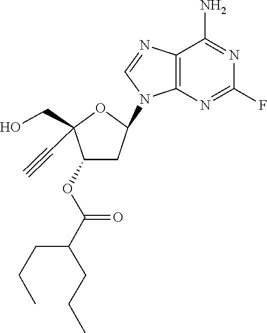 [SMILES: C#C[C@]1(CO)O[C@@H](n2cnc3c(N)nc(F)nc32)C[C@@H]1OC(=O)C(CCC)CCC] | (2R,3S,5R)-5-(6-amino-2-fluoro-9H- purin-9-yl)-2-ethynyl-2- (hydroxymethyl)tetrahydrofuran-3-yl 2-propylpentanoate |
| 6 | 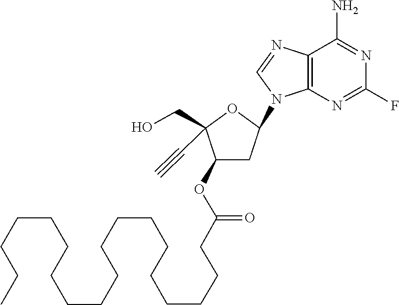 [SMILES: C#C[C@]1(CO)O[C@@H](n2cnc3c(N)nc(F)nc32)C[C@H]1OC(=O)CCCCCCCCCCCCCCCCCCC] | (2R,3S,5R)-5-(6-amino-2-fluoro-9H- purin-9-yl)-2-ethynyl-2- (hydroxymethyl)tetrahydrofuran-3-yl icosanoate |
| 7 | 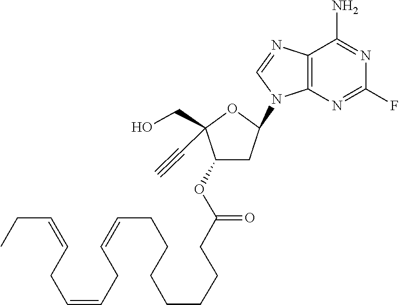 [SMILES: C#C[C@]1(CO)O[C@@H](n2cnc3c(N)nc(F)nc32)C[C@@H]1OC(=O)CCCCCCC/C=C\C/C=C\C/C=C\CC] | (9Z,12Z,15Z)-(2R,3S,5R)-5-(6-amino-2- fluoro-9H-purin-9-yl)-2-ethynyl-2- (hydroxymethyl)tetrahydrofuran-3-yl octadeca-9,12,15-trienoate |
| 8 | 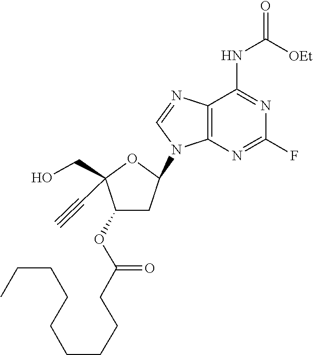 [SMILES: C#C[C@]1(CO)O[C@@H](n2cnc3c(NC(=O)OCC)nc(F)nc32)C[C@@H]1OC(=O)CCCCCCCCC] | (2R,3S,5R)-5-(6-((ethoxycarbonyl)amino)- 2-fluoro-9H-purin-9-yl)-2-ethynyl-2- (hydroxymethyl)tetrahydrofuran-3-yl decanoate |
| 9 | 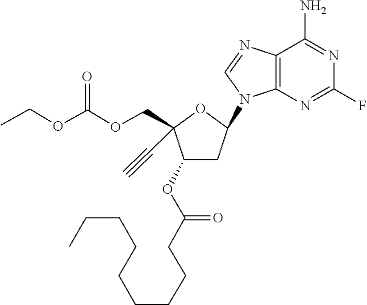 [SMILES: C#C[C@]1(COC(=O)OCC)O[C@@H](n2cnc3c(N)nc(F)nc32)C[C@@H]1OC(=O)CCCCCCCCC] | (2R,3S,5R)-5-(6-amino-2-fluoro-9H- purin-9-yl)-2- (((ethoxycarbonyl)oxy)methyl)-2- ethynyltetrahydrofuran-3-yl decanoate |
| 10 | 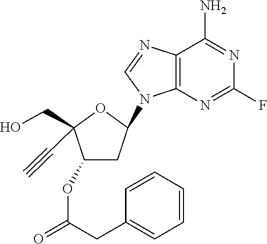 [SMILES: C#C[C@]1(CO)O[C@@H](n2cnc3c(N)nc(F)nc32)C[C@@H]1OC(=O)Cc1ccccc1] | (2R,3S,5R)-5-(6-amino-2-fluoro-9H- purin-9-yl)-2-ethynyl-2- (hydroxymethyl)tetrahydrofuran-3-yl 2-phenylacetate |
| 11 | 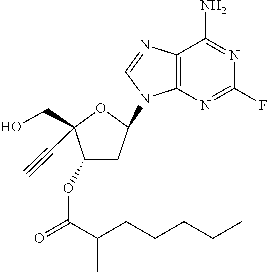 [SMILES: C#C[C@]1(CO)O[C@@H](n2cnc3c(N)nc(F)nc32)C[C@@H]1OC(=O)C(C)CCCCC] | (2R,3S,5R)-5-(6-amino-2-fluoro-9H- purin-9-yl)-2-ethynyl-2- (hydroxymethyl)tetrahydrofuran-3-yl 2- methylheptanoate |
| 12 | 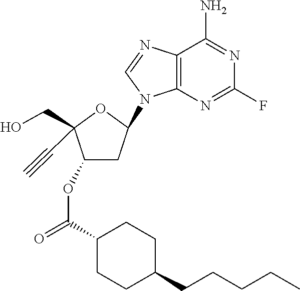 [SMILES: C#C[C@]1(CO)O[C@@H](n2cnc3c(N)nc(F)nc32)C[C@@H]1OC(=O)[C@H]1CC[C@H](CCCCC)CC1] | (2R,3S,5R)-5-(6-amino-2-fluoro-9H- purin-9-yl)-2-ethynyl-2- (hydroxymethyl)tetrahydrofuran-3-yl (1s,4S)-4-pentylcyclohexane-1-carboxylate |
| 13 | 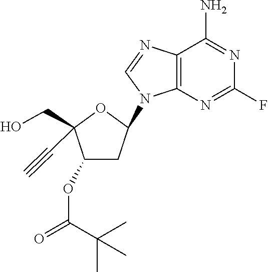 [SMILES: C#C[C@]1(CO)O[C@@H](n2cnc3c(N)nc(F)nc32)C[C@@H]1OC(=O)C(C)(C)C] | (2R,3S,5R)-5-(6-amino-2-fluoro-9H- purin-9-yl)-2-ethynyl-2- (hydroxymethyl)tetrahydrofuran-3-yl pivalate |
| 14 | 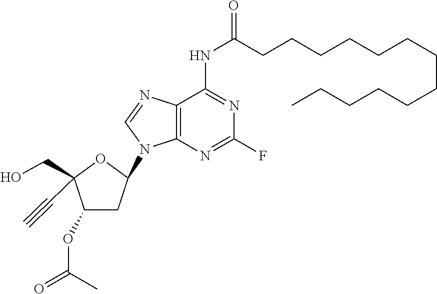 [SMILES: C#C[C@]1(CO)O[C@@H](n2cnc3c(NC(=O)CCCCCCCCCCCCC)nc(F)nc32)C[C@@H]1OC(C)=O] | (2R,3S,5R)-2-ethynyl-5-(2-fluoro-6- tetradecanamido-9H-purin-9-yl)-2- (hydroxymethyl)tetrahydrofuran-3-yl acetate |
| 15 | 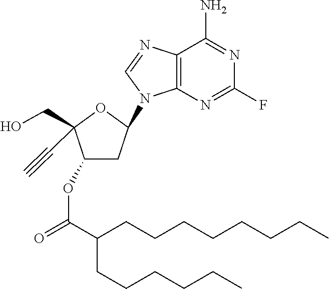 [SMILES: C#C[C@]1(CO)O[C@@H](n2cnc3c(N)nc(F)nc32)C[C@@H]1OC(=O)C(CCCCCC)CCCCCCCC] | (2R,3S,5R)-5-(6-amino-2-fluoro-9H- purin-9-yl)-2-ethynyl-2- (hydroxymethyl)tetrahydrofuran-3-yl 2- hexyldecanoate |
| 16 | 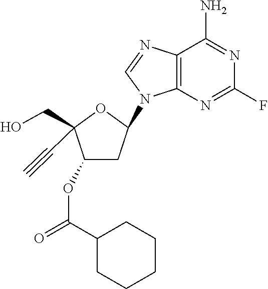 [SMILES: C#C[C@]1(CO)O[C@@H](n2cnc3c(N)nc(F)nc32)C[C@@H]1OC(=O)C1CCCCC1] | (2R,3S,5R)-5-(6-amino-2-fluoro-9H- purin-9-yl)-2-ethynyl-2- (hydroxymethyl)tetrahydrofuran-3-yl cyclohexanecarboxylate |
| 17 | 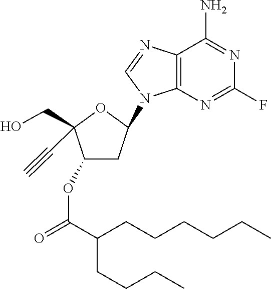 [SMILES: C#C[C@]1(CO)O[C@@H](n2cnc3c(N)nc(F)nc32)C[C@@H]1OC(=O)C(CCCC)CCCCCC] | (2R,3S,5R)-5-(6-amino-2-fluoro-9H- purin-9-yl)-2-ethynyl-2- (hydroxymethyl)tetrahydrofuran-3-yl 2-butyloctanoate |
| 18 | 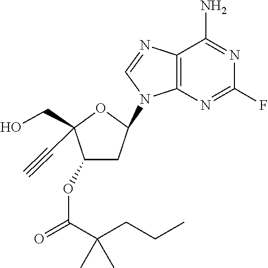 [SMILES: C#C[C@]1(CO)O[C@@H](n2cnc3c(N)nc(F)nc32)C[C@@H]1OC(=O)C(C)(C)CCC] | (2R,3S,5R)-5-(6-amino-2-fluoro-9H- purin-9-yl)-2-ethynyl-2- (hydroxymethyl)tetrahydrofuran-3-yl 2,2-dimethylpentanoate |
| 19 | 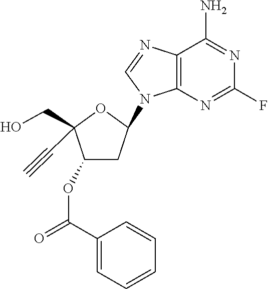 [SMILES: C#C[C@]1(CO)O[C@@H](n2cnc3c(N)nc(F)nc32)C[C@@H]1OC(=O)c1ccccc1] | (2R,3S,5R)-5-(6-amino-2-fluoro-9H- purin-9-yl)-2-ethynyl-2- (hydroxymethyl)tetrahydrofuran-3-yl benzoate |
| 20 | 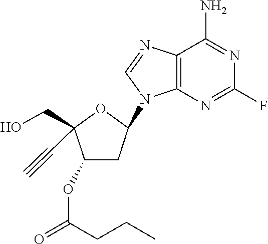 [SMILES: C#C[C@]1(CO)O[C@@H](n2cnc3c(N)nc(F)nc32)C[C@@H]1OC(=O)CCC] | (2R,3S,5R)-5-(6-amino-2-fluoro-9H- purin-9-yl)-2-ethynyl-2- (hydroxymethyl)tetrahydrofuran-3-yl butyrate |
| 21 | 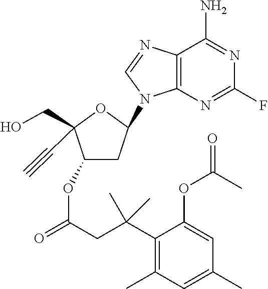 [SMILES: C#C[C@]1(CO)O[C@@H](n2cnc3c(N)nc(F)nc32)C[C@@H]1OC(=O)CC(C)(C)c1c(C)cc(C)cc1OC(C)=O] | (2R,3S,5R)-5-(6-amino-2-fluoro-9H- purin-9-yl)-2-ethynyl-2- (hydroxymethyl)tetrahydrofuran-3-yl 3-(2-acetoxy-4,6- dimethylphenyl)-3-methylbutanoate |
| 22 | 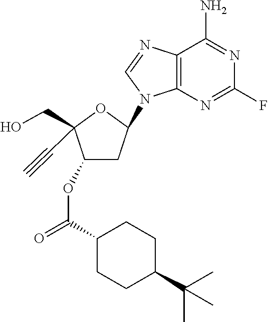 [SMILES: C#C[C@]1(CO)O[C@@H](n2cnc3c(N)nc(F)nc32)C[C@@H]1OC(=O)[C@H]1CC[C@H](C(C)(C)C)CC1] | (2R,3S,5R)-5-(6-amino-2-fluoro-9H- purin-9-yl)-2-ethynyl-2- (hydroxymethyl)tetrahydrofuran-3-yl (1r,4S)-4-(tert-butyl)cyclohexane- 1-carboxylate |
| 23 | 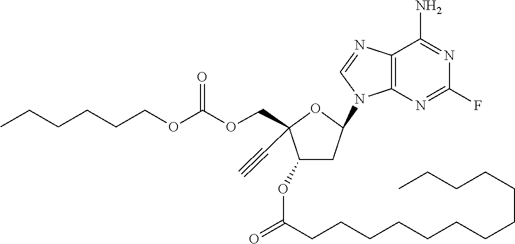 [SMILES: C#C[C@]1(COC(=O)OCCCCCC)O[C@@H](n2cnc3c(N)nc(F)nc32)C[C@@H]1OC(=O)CCCCCCCCCCCCC] | (2R,3S,5R)-5-(6-amino-2-fluoro-9H- purin-9-yl)-2-ethynyl-2- ((((hexyloxy)carbonyl)oxy)methyl) tetrahydrofuran-3-yl tetradecanoate |
| 24 | 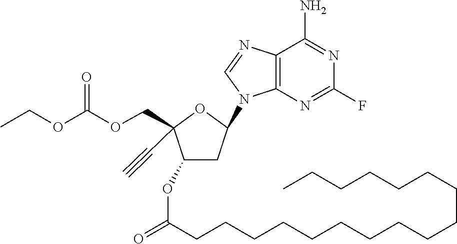 [SMILES: C#C[C@]1(COC(=O)OCC)O[C@@H](n2cnc3c(N)nc(F)nc32)C[C@@H]1OC(=O)CCCCCCCCCCCCCCCCC] | (2R,3S,5R)-5-(6-amino-2-fluoro-9H- purin-9-yl)-2- (((ethoxycarbonyl)oxy)methyl)- 2-ethynyltetrahydrofuran-3-yl stearate |
| 25 | 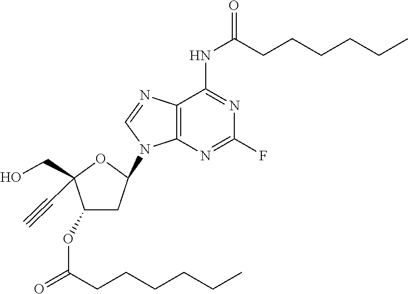 [SMILES: C#C[C@]1(CO)O[C@@H](n2cnc3c(NC(=O)CCCCCC)nc(F)nc32)C[C@@H]1OC(=O)CCCCCC] | (2R,3S,5R)-2-ethynyl-5-(2-fluoro-6- heptanamido-9H-purin-9-yl)-2- (hydroxymethyl)tetrahydrofuran-3-yl heptanoate |
| 26 | 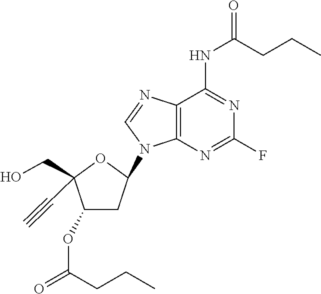 [SMILES: C#C[C@]1(CO)O[C@@H](n2cnc3c(NC(=O)CCC)nc(F)nc32)C[C@@H]1OC(=O)CCC] | (2R,3S,5R)-5-(6-butyramido-2-fluoro-9H- purin-9-yl)-2-ethynyl-2- (hydroxymethyl)tetrahydrofuran-3-yl butyrate |
| 27 | 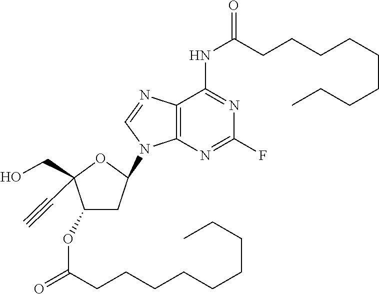 [SMILES: C#C[C@]1(CO)O[C@@H](n2cnc3c(NC(=O)CCCCCCCCC)nc(F)nc32)C[C@@H]1OC(=O)CCCCCCCCC] | (2R,3S,5R)-5-(6-decanamido-2-fluoro-9H- purin-9-yl)-2-ethynyl-2- (hydroxymethyl)tetrahydrofuran-3-yl decanoate |
| 28 | 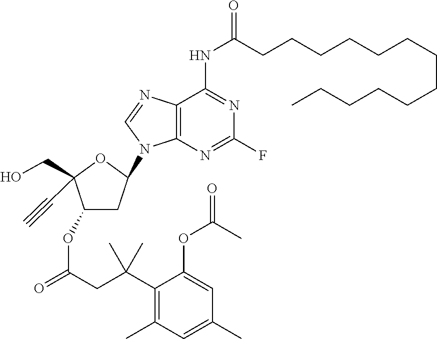 [SMILES: C#C[C@]1(CO)O[C@@H](n2cnc3c(NC(=O)CCCCCCCCCCCCC)nc(F)nc32)C[C@@H]1OC(=O)CC(C)(C)c1c(C)cc(C)cc1OC(C)=O] | (2R,3S,5R)-2-ethynyl-5- (2-fluoro-6-tetradecanamido-9H-purin-9-yl)- 2-(hydroxymethyl)tetrahydrofuran-3-yl 3- (2-acetoxy-4,6-dimethylphenyl)- 3-methylbutanoate |
| 29 | 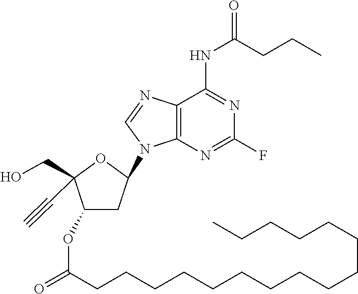 [SMILES: C#C[C@]1(CO)O[C@@H](n2cnc3c(NC(=O)CCC)nc(F)nc32)C[C@@H]1OC(=O)CCCCCCCCCCCCCCCC] | (2R,3S,5R)-5-(6-butyramido-2-fluoro-9H- purin-9-yl)-2-ethynyl-2- (hydroxymethyl)tetrahydrofuran-3-yl heptadecanoate |
| 30 | 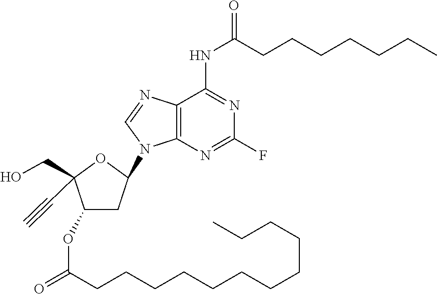 [SMILES: C#C[C@]1(CO)O[C@@H](n2cnc3c(NC(=O)CCCCCCC)nc(F)nc32)C[C@@H]1OC(=O)CCCCCCCCCCCC] | (2R,3S,5R)-2-ethynyl-5- (2-fluoro-6-octanamido-9H-purin-9-yl)- 2-(hydroxymethyl)tetrahydrofuran- 3-yl tridecanoate |
| 31 | 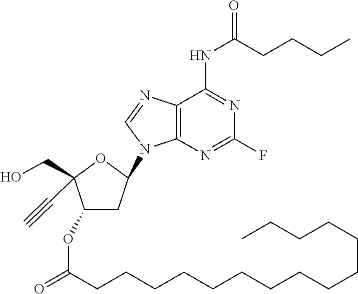 [SMILES: C#C[C@]1(CO)O[C@@H](n2cnc3c(NC(=O)CCCC)nc(F)nc32)C[C@@H]1OC(=O)CCCCCCCCCCCCCCC] | (2R,3S,5R)-2-ethynyl-5- (2-fluoro-6-pentanamido-9H-purin-9-yl)-2- (hydroxymethyl)tetrahydrofuran-3-yl palmitate |
| 32 | 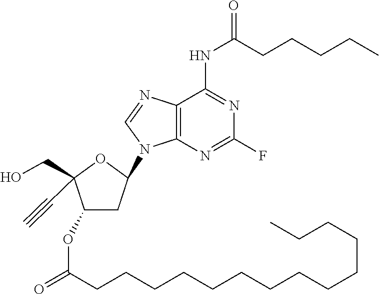 [SMILES: C#C[C@]1(CO)O[C@@H](n2cnc3c(NC(=O)CCCCC)nc(F)nc32)C[C@@H]1OC(=O)CCCCCCCCCCCCCC] | (2R,3S,5R)-2-ethynyl-5- (2-fluoro-6-hexanamido-9H-purin-9-yl)-2- (hydroxymethyl)tetrahydrofuran-3-yl pentadecanoate |
| 33 | 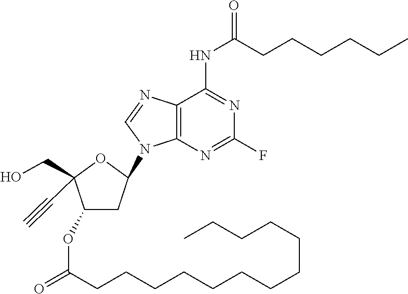 [SMILES: C#C[C@]1(CO)O[C@@H](n2cnc3c(NC(=O)CCCCCC)nc(F)nc32)C[C@@H]1OC(=O)CCCCCCCCCCCCC] | (2R,3S,5R)-2-ethynyl-5- (2-fluoro-6-heptanamido-9H-purin-9-yl)-2- (hydroxymethyl)tetrahydrofuran-3-yl tetradecanoate |

and a pharmaceutically acceptable salt thereof.

In one embodiment, the present invention encompasses each individual compound listed in the above Table 1, or a pharmaceutically acceptable salt thereof. As an example, in one preferred embodiment, the invention relates to a compound of the formula:

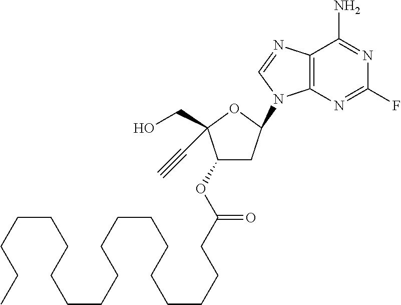 [SMILES: C#C[C@]1(CO)O[C@@H](n2cnc3c(N)nc(F)nc32)C[C@@H]1OC(=O)CCCCCCCCCCCCCCCCCCC]

which is (2R,3S,5R)-5-(6-amino-2-fluoro-9H-purin-9-yl)-2-ethynyl-2-(hydroxymethyl) tetrahydrofuran-3-yl icosanoate, and pharmaceutically acceptable salts thereof. Most preferably, (2R,3S,5R)-5-(6-amino-2-fluoro-9H-purin-9-yl)-2-ethynyl-2-(hydroxymethyl) tetrahydrofuran-3-yl icosanoate is present as a free base.

In accordance with one embodiment of the present invention, there is provided a pharmaceutical composition comprising a compound of Formulae (I), (Ia), (II) and (IIa) or a pharmaceutically acceptable salt thereof, and a pharmaceutically acceptable excipient. In a further embodiment, the compound is present in amorphous form. In a further embodiment, the pharmaceutical composition is in a tablet form. In a further embodiment, the pharmaceutical composition is in parenteral form. In a further embodiment, the compound is present as a spray dried dispersion.

In accordance with one embodiment of the present invention, there is provided a method of treating an HIV infection in a subject comprising administering to the subject a compound of Formulae (I), (Ia), (II) and (IIa) or a pharmaceutically acceptable salt thereof.

In accordance with one embodiment of the present invention, there is provided a method of treating an HIV infection in a subject comprising administering to the subject a pharmaceutical composition as described herein.

In accordance with one embodiment of the present invention, there is provided a method of preventing an HIV infection in a subject at risk for developing an HIV infection, comprising administering to the subject a compound of Formulae (I), (Ia), (II) and (IIa) or a pharmaceutically acceptable salt thereof.

In accordance with one embodiment of the present invention, there is provided the use of a compound of Formulae (I), (Ia), (II) and (IIa) in the manufacture of a medicament for treating an HIV infection.

In accordance with one embodiment of the present invention, there is provided the use of a compound of Formulae (I), (Ia), (II) and (IIa) in the manufacture of a medicament for preventing an HIV infection.

In accordance with one embodiment of the present invention, there is provided a compound according to Formulae (I), (Ia), (II) and (IIa) for use in treating an HIV infection.

In accordance with one embodiment of the present invention, there is provided a compound according to Formulae (I), (Ia), (II) and (IIa) for use in preventing an HIV infection.

In accordance with one embodiment of the present invention, there is provided a method of preventing an HIV infection in a subject at risk for developing an HIV infection, comprising administering to the subject a pharmaceutical composition as described herein.

Furthermore, the compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically ordiastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and I isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

In another embodiment of the invention, there is provided a compound of Formulae (I), (Ia),-(II) and (IIa) wherein the compound or salt of the compound is used in the manufacture of a medicament for use in the treatment of an HIV infection in a human.

In another embodiment of the invention, there is provided a compound of Formulae (I), (Ia),-(II) and (IIa) wherein the compound or salt of the compound is used in the manufacture of a medicament for use in the prevention of an HIV infection in a human.

In one embodiment, the pharmaceutical formulation containing a compound of Formulae (I), (Ia),-(II) and (IIa) or a salt thereof is a formulation adapted for parenteral administration. In another embodiment, the formulation is a long-acting parenteral formulation. In a further embodiment, the formulation is a nano-particle formulation.

The compounds of the present invention and their salts, solvates, or other pharmaceutically acceptable derivatives thereof, may be employed alone or in combination with other therapeutic agents. Therefore, in other embodiments, the methods of treating and/or preventing an HIV infection in a subject may in addition to administration of a compound of Formulae (I), (Ia),-(II) and (IIa) further comprise administration of one or more additional pharmaceutical agents active against HIV.

In such embodiments, the one or more additional agents active against HIV is selected from the group consisting of zidovudine, didanosine, lamivudine, zalcitabine, abacavir, stavudine, adefovir, adefovirdipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir.cabotegravir, vicriviroc (Sch-C), Sch-D, TAK779, maraviroc, TAK449, didanosine, tenofovir, lopinavir, and darunavir.

As such, the compounds of the present invention of Formulae (I), (Ia), (II) and (IIa) and any other pharmaceutically active agent(s) may be administered together or separately and, when administered separately, administration may occur simultaneously or sequentially, in any order. The amounts of the compounds of Formulae (I), (Ia), (II) and (IIa) of the present invention and the other pharmaceutically active agent(s) and the relative timings of administration will be selected in order to achieve the desired combined therapeutic effect. The administration in combination of a compound of the present invention of Formulae (I), (Ia), (II) and (IIa) and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of Formula (I) or (II) salts thereof and the other pharmaceutically active agent(s) and the relative timings of administration will be selected in order to achieve the desired combined therapeutic effect.

In addition, the compounds of the present invention of Formulae (I), (Ia), (II) and (IIa) may be used in combination with one or more other agents that may be useful in the prevention or treatment of HIV. Examples of such agents include:

Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, and similar agents;
Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, GSK2248761, TMC-278, TMC-125, etravirine, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Entry, attachment and fusion inhibitors such as enfuvirtide (T-20), T-1249, PRO-542, PRO-140, TNX-355, BMS-806, BMS-663068, BMS-626529, 5-Helix and similar agents;
Inteqrase inhibitors such as raltegravir, elvitegravir, dolutegravir, bictegravir, cabotegravir and similar agents;
Maturation inhibitors such as PA-344 and PA-457, and similar agents; and
CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

Other combinations may be used in conjuction with the compounds of the present invention, e.g., Biktarvy® (Bictegravir/Emtricitabine/Tenofovir/Alafenamide) made commercially available by Gilead Sciences

Further examples where the compounds of the present invention may be used in combination with one or more agents useful in the prevention or treatment of HIV are found in Table 2.

TABLE 2

| | | | |
|---|---|---|---|
| FDA | Brand | | |
| Approval | Name | Generic Name | Manufacturer |

Nucleoside
Reverse
Transcriptase
Inhibitors (NRTIs)

| | | | |
|---|---|---|---|
| 1987 | Retrovir | zidovudine, | GlaxoSmithKline |
| | | azidothymidine, | |
| | | AZT, ZDV | |
| 1991 | Videx | didanosine, | Bristol-Myers |
| | | dideoxyinosine, ddl | Squibb |
| 1992 | Hivid | zalcitabine, | Roche |
| | | dideoxycytidine, ddC | Pharmaceuticals |
| 1994 | Zerit | stavudine, d4T | Bristol-Myers |
| | | | Squibb |
| 1995 | Epivir | lamivudine, 3TC | GlaxoSmithKline |
| 1997 | Combivir | lamivudine + | GlaxoSmithKline |
| | | zidovudine | |
| 1998 | Ziagen | abacavir sulfate, ABC | GlaxoSmithKline |
| 2000 | Trizivir | abacavir + | GlaxoSmithKline |
| | | lamivudine + | |
| | | zidovudine | |
| 2000 | Videx EC | enteric coated | Bristol-Myers |
| | | didanosine, ddl EC | Squibb |
| 2001 | Viread | tenofovir disoproxil | Gilead Sciences |
| | | fumarate, TDF | |
| 2003 | Emtriva | emtricitabine, FTC | Gilead Sciences |
| 2004 | Epzicom | abacavir + lamivudine | GlaxoSmithKline |
| 2004 | Truvada | emtricitabine + | Gilead Sciences |
| | | tenofovir disoproxil | |
| | | fumarate | |

Non-Nucleosides
Reverse
Transcriptase
Inhibitors
(NNRTIs)

| | | | |
|---|---|---|---|
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | Etravirine | Tibotec |
| | | | Therapeutics |

Protease
Inhibitors (PIs)

| | | | |
|---|---|---|---|
| 1995 | Invirase | saquinavir mesylate, | Roche |
| | | SQV | Pharmaceuticals |
| 1996 | Norvir | ritonavir, RTV | Abbott |
| | | | Laboratories |
| 1996 | Crixivan | indinavir, IDV | Merck |
| 1997 | Viracept | nelfinavir mesylate, | Pfizer |
| | | NFV | |
| 1997 | Fortovase | saquinavir (no longer | Roche |
| | | marketed) | Pharmaceuticals |
| 1999 | Agenerase | amprenavir, APV | GlaxoSmithKline |
| 2000 | Kaletra | lopinavir + ritonavir, | Abbott |
| | | LPV/RTV | Laboratories |
| 2003 | Reyataz | atazanavir sulfate, | Bristol-Myers |
| | | ATV | Squibb |
| 2003 | Lexiva | fosamprenavir | GlaxoSmithKline |
| | | calcium, FOS-APV | |
| 2005 | Aptivus | tripranavir, TPV | Boehringer |
| | | | Ingelheim |
| 2006 | Prezista | Darunavir | Tibotec |
| | | | Therapeutics |

Fusion Inhibitors

| | | | |
|---|---|---|---|
| 2003 | Fuzeon | Enfuvirtide, T-20 | Roche |
| | | | Pharmaceuticals |
| | | | & Trimeris |

Entry Inhibitors

| | | | |
|---|---|---|---|
| 2007 | Selzentry | Maraviroc | Pfizer |

Integrase
Inhibitors

| | | | |
|---|---|---|---|
| 2007 | Isentress | Raltegravir | Merck |
| 2013 | Tivicay | Dolutegravir | ViiV Healthcare |
| — | — | Cabotegravir | |

The scope of combinations of compounds of this invention with HIV agents is not limited to those mentioned above, but includes in principle any combination with any pharmaceutical composition useful for the treatment and/or prevention of HIV. As noted, in such combinations the compounds of the present invention and other HIV agents may be administered separately or in conjunction. In addition, one agent may be prior to, concurrent to, or subsequent to the administration of other agent(s).

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir, GS-9350, and SPI-452. Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott park, Illinois, as Norvir. Ritonavir is an HIV protease inhibitor indicated with other antiretroviral agents for the treatment of HIV infection. Ritonavir also inhibits P450 mediated drug metabolism as well as the P-gycoprotein (Pgp) cell transport system, thereby resulting in increased concentrations of active compound within the organism.

GS-9350 is a compound being developed by Gilead Sciences of Foster City Calif. as a pharmacological enhancer.
SPI-452 is a compound being developed by Sequoia Pharmaceuticals of Gaithersburg, Md., as a pharmacological enhancer.

In one embodiment of the present invention, a compound of Formulae (I), (Ia), (II) and (IIa) are used in combination with ritonavir. In one embodiment, the combination is an oral fixed dose combination. In another embodiment, the compound of Formulae (I), (Ia), (II) and (IIa) are formulated as a long acting parenteral injection and ritonavir is formulated as an oral composition. In one embodiment, a kit containing the compounds of Formulae (I), (Ia), (II) and (IIa) are formulated as a long acting parenteral injection and ritonavir formulated as an oral composition. In another embodiment, the compounds of Formulae (I), (Ia), (II) and (IIa) are formulated as a long acting parenteral injection and ritonavir is formulated as an injectable composition. In one embodiment, a kit containing the compounds of Formulae (I), (Ia), (II) and (IIa) are formulated as a long acting parenteral injection and ritonavir formulated as an injectable composition.

In another embodiment of the present invention, a compound of Formulae (I), (Ia), (II) and (IIa) are in combination with GS-9350. In one embodiment, the combination is an oral fixed dose combination. In another embodiment, the compound of Formulae (I), (Ia), (II) and (IIa) are formulated as a long acting parenteral injection and GS-9350 is formulated as an oral composition. In one embodiment, there is provided a kit containing the compound of Formula (I), (Ia), (II) and (IIa) is formulated as a long acting parenteral injection and GS-9350 formulated as an oral composition. In another embodiment, the compound of Formulae (I), (Ia), (II) and (IIa) are formulated as a long acting parenteral injection and GS-9350 is formulated as an injectable composition. In one embodiment, is a kit containing the compound of Formulae (I), (Ia), (II) and (IIa) are formulated as a long acting parenteral injection and GS-9350 formulated as an injectable composition.

In one embodiment of the present invention, a compound of Formulae (I), (Ia), (II) and (IIa) are used in combination with SPI-452. In one embodiment, the combination is an oral fixed dose combination. In another embodiment, the compound of Formulae (I), (Ia), (II) and (IIa) are formulated as a long acting parenteral injection and SPI-452 is formulated as an oral composition. In one embodiment, there is provided a kit containing the compound of Formula (I), (Ia), (II) and (IIa) formulated as a long acting parenteral injection and SPI-452 formulated as an oral composition. In another embodiment, the compound of Formulae (I), (Ia), (II) and (IIa) are formulated as a long acting parenteral injection and SPI-452 is formulated as an injectable composition. In one embodiment, there is provided a kit containing the compound of Formulae (I), (Ia), (II) and (IIa) formulated as a long acting parenteral injection and SPI-452 formulated as an injectable composition.

In one embodiment of the present invention, a compound of Formulae (I), (Ia), (II) and (IIa) are used in combination with compounds which are found in previously filed PCT/CN2011/0013021, which is herein incorporated by reference.

The above other therapeutic agents, when employed in combination with the chemical entities described herein, may be used, for example, in those amounts indicated in the Physicians' Desk Reference (PDR) or as otherwise determined by one of ordinary skill in the art.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa).

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa), wherein said virus is an HIV virus. In some embodiments, the HIV virus is the HIV-1 virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa) further comprising administration of a therapeutically effective amount of one or more agents active against an HIV virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa), further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa).

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa), wherein said virus is an HIV virus. In some embodiments, the HIV virus is the HIV-1 virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa), further comprising administration of a therapeutically effective amount of one or more agents active against an HIV virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa) further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In further embodiments, the compound of the present invention of Formulae (I), (Ia), (II) and (IIa) or a pharmaceutically acceptable salt thereof, is selected from the group of compounds set forth in Table 1 above.

The compounds of Table 1 were synthesized according to the Synthetic Methods, General Schemes, and the Examples described below.

In another embodiment, there is provided a pharmaceutical composition comprising a pharmaceutically acceptable diluent and a therapeutically effective amount of a compound of Formulae (I), (Ia), (II) and (IIa) or a pharmaceutically acceptable salt thereof.

In certain embodiments, the compound(s) of the present invention, or a pharmaceutically acceptable salt thereof, is chosen from the compounds set forth in Table 1.

The compounds of the present invention can be supplied in the form of a pharmaceutically acceptable salt. The terms “pharmaceutically acceptable salt” refer to salts prepared from pharmaceutically acceptable inorganic and organic acids and bases. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds according to Formulae (I), (Ia), (II) and (IIa) may be prepared. These pharmaceutically acceptable salts may be prepared in situ during the final isolation and purification of the compound, or by separately reacting the purified compound in its free acid or free base form with a suitable base or acid, respectively.

Illustrative pharmaceutically acceptable acid salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, p-hydroxybutyric, galactaric and galacturonic acids. Preferred pharmaceutically acceptable salts include the salts of hydrochloric acid and trifluoroacetic acid.

Illustrative pharmaceutically acceptable inorganic base salts of the compounds of the present invention include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary base salts include aluminum, calcium, lithium, magnesium, potassium, sodium and zinc. Other exemplary base salts include the ammonium, calcium, magnesium, potassium, and sodium salts. Still other exemplary base salts include, for example, hydroxides, carbonates, hydrides, and alkoxides including NaOH, KOH, Na₂CO₃, K₂CO₃, NaH, and potassium-t-butoxide.

Salts derived from pharmaceutically acceptable organic non-toxic bases include salts of primary, secondary, and tertiary amines, including in part, trimethylamine, diethylamine, N, N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionisation in the salt may vary from completely ionised to almost non-ionised. Lists of suitable salts are found in Remington's Pharmaceutical Sciences. 17th ed., Mack Publishing Company, Easton, Pa., 1985, p. 1418, the disclosure of which is hereby incorporated by reference only with regards to the lists of suitable salts.

The compounds of Formula (I), (Ia), (II) and (IIa) of the invention may exist in both unsolvated and solvated forms. The term ‘solvate’ comprises the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

Compounds of Formulae (I), (Ia), (II) and (IIa) containing one or more asymmetric carbon atoms can exist as two or more stereoisomers. Where a compound of Formulae (I), (Ia), (II) and (IIa) contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formula (I), (II), (Ia) and (IIa) including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula (I) or (II) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of Formula (I), (Ia), (II) and (IIa) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formulae (I), (Ia), (II) and (IIa), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds of Formulae (I), (Ia), (II) and (IIa) can generally be prepared by conventional techniques known to those skilled in the art or by processes analogous to those described herein using an appropriate isotopically-labelled reagents in place of the non-labelled reagent previously employed.

The compounds of the present invention may be administered as prodrugs. In one embodiment, the compounds of the invention are prodrugs of 4′-ethynyl-2-fluoro-2′-deoxyadenosine (EFdA) disclosed e.g., in U.S. Pat. No. 7,339,053, which is a nucleoside reverse transcriptase inhibitor of the formula:

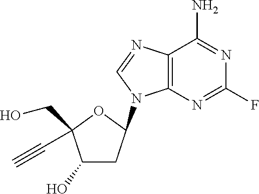 [SMILES: C#C[C@]1(CO)O[C@@H](n2cnc3c(N)nc(F)nc32)C[C@@H]1O]

One preferred prodrug is (2R,3S,5R)-5-(6-amino-2-fluoro-9H-purin-9-yl)-2-ethynyl-2-(hydroxymethyl) tetrahydrofuran-3-yl icosanoate, and pharmaceutically acceptable salts thereof. The prodrugs are useful in that they are capable of modulating physicochemical properties, facilitating multiple dosing paradigms and improving pharmacokinetic and/or pharmacodynamic profiles of the active parent (EfdA). More specifically, EFdA has a relatively high aqueous solubility, rendering it unsuitable for slow release, long acting, parenteral dosing. Advantageously, prodrugs of EFdA of the invention are capable of having substantially reduced aqueous solubilities, that in some cases, may facilitate a slow release, parenteral dosing modality. Additionally, prodrugs of EFdA, of the invention may also reduce or eliminate undesirable injection site reactions associated with high localized concentrations of EFdA that occur upon parenteral dosing of EFdA itself. Moreover, prodrugs of EFdA of the invention may also, in some cases, confer an enhancement in antiviral persistence as compared to EFdA.

Administration of the chemical entities and combinations of entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used. Examples of dosing include, without limitation, once every seven days for oral, once every eight weeks for intramuscular, or once every six months for subcutaneous.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition may comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used the route and form of administration, and other factors. The drug can be administered more than once a day, such as once or twice a day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %.

In various embodiments, pharmaceutical compositions of the present invention encompass compounds of Formulae (I), (Ia), (II) and (IIa), salts thereof, and combinations of the above.

### Synthetic Methods

The methods of synthesis may employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers ordiastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein may or take place at atmospheric pressure, generally within a temperature range from −78° C. to 200° C. Further, except as employed in the Example or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (ordichloromethane), diethyl ether, methanol, N-methylpyrrolidone (“NMP”), pyridine and the like.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### EXAMPLE AND GENERAL SYNTHESIS

The following examples and prophetic syntheses schemes serve to more fully describe the manner of making and using the above-described invention. It is understood that this in no way serve to limit the true scope of the invention, but rather is presented for illustrative purposes. Unless otherwise specified, the following abbreviations have the following meanings. If an abbreviation is not defined, it has its generally accepted meaning.
aq.=Aqueous
μL=Microliters
μM=Micromolar
NMR=nuclear magnetic resonance
Boc=tert-butoxycarbonyl
Br=Broad
Cbz=Benzyloxycarbonyl
D=Doublet
Δ=chemical shift
° C.=degrees celcius
DCM=Dichloromethane
Dd=doublet of doublets
   DMAP=4-dimethylaminopyridine
DMEM=Dulbeco's Modified Eagle's Medium
DMF=N,N-dimethylformamide
DMSO=Dimethylsulfoxide
EtOAc=ethyl acetate
G=Gram
h or hr=Hours
HCV=hepatitus C virus
HPLC=high performance liquid chromatography
Hz=Hertz
IU=International Units
IC₅₀=inhibitory concentration at 50% inhibition
J=coupling constant (given in Hz unless otherwise indicated)
M=Multiplet
M=Molar
M+H⁺=parent mass spectrum peak plus H⁺
Mg=Milligram
Min=Minutes
mL=Milliliter
mM=Millimolar
Mmol=Millimole
MS=mass spectrum
Nm=Nanomolar
Ppm=parts per million
q.s.=sufficient amount
S=Singlet
RT=room temperature
sat.=Saturated
T=Triplet
TBDPS=tert-butyldiphenylsilyl
TEA=triethylamine
TFA=trifluoroacetic acid
THF=tetrahydrofuran
TMS=trimethyl silyl
   Various compounds of the invention may be made, in certain embodiment, byway of the general prophetic syntheses schemes I through III set forth below:

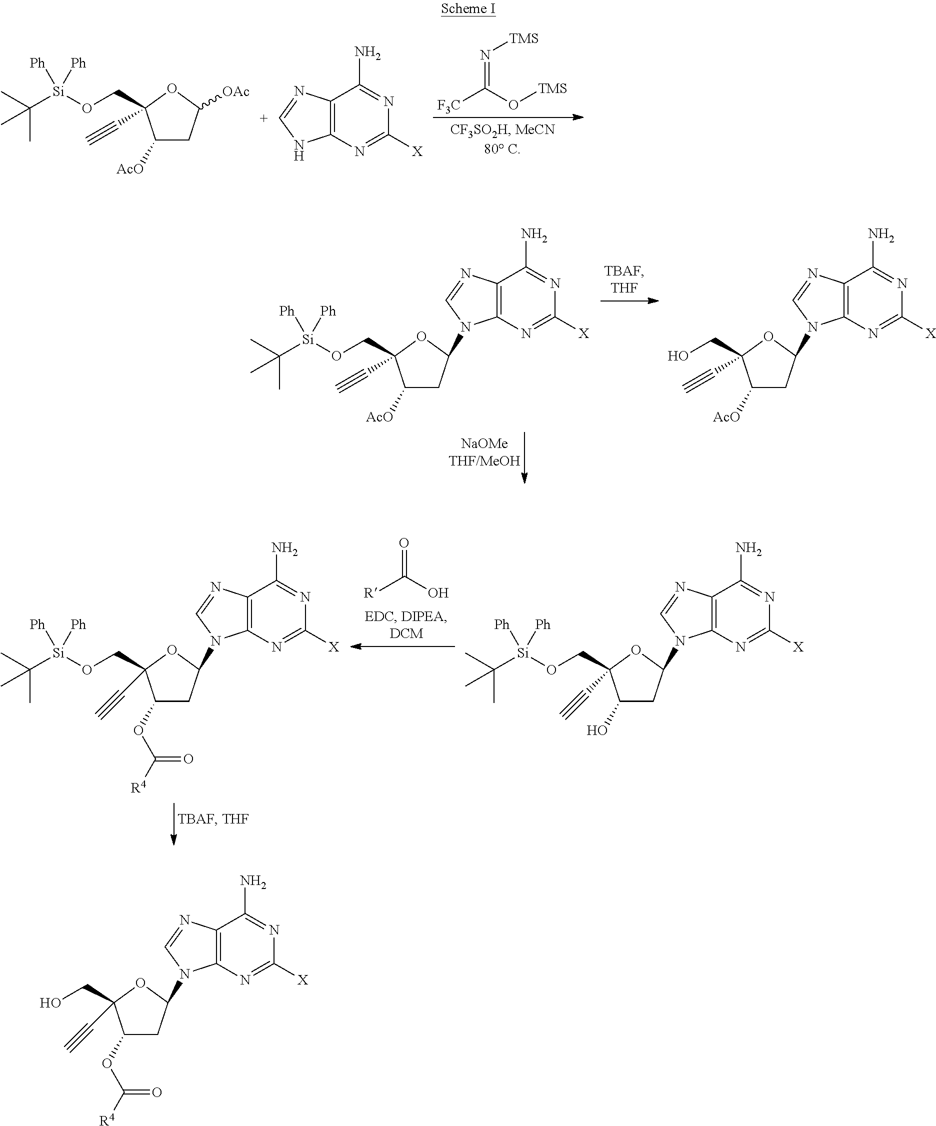

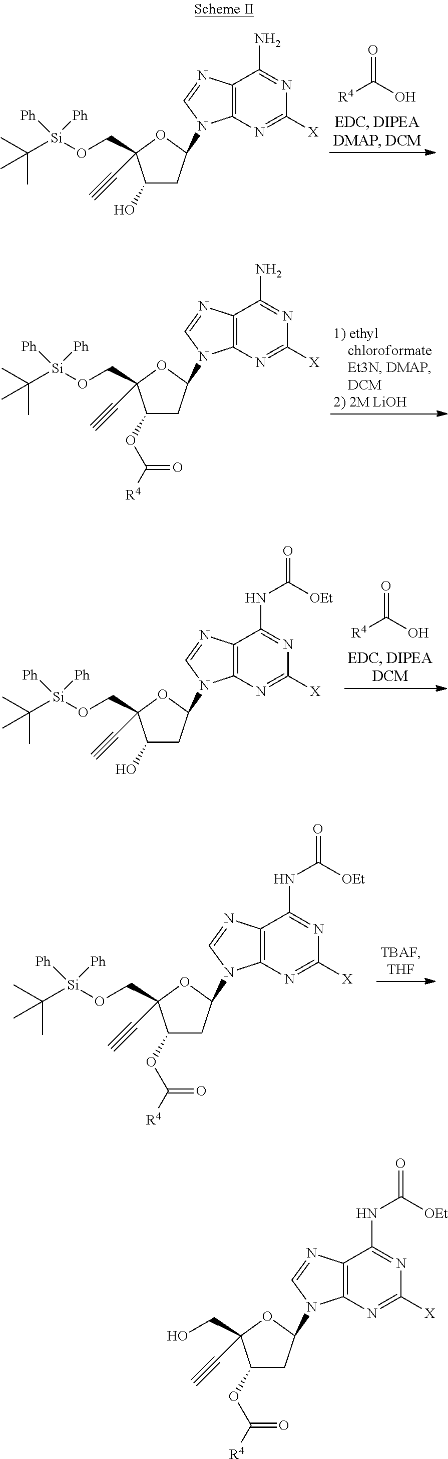

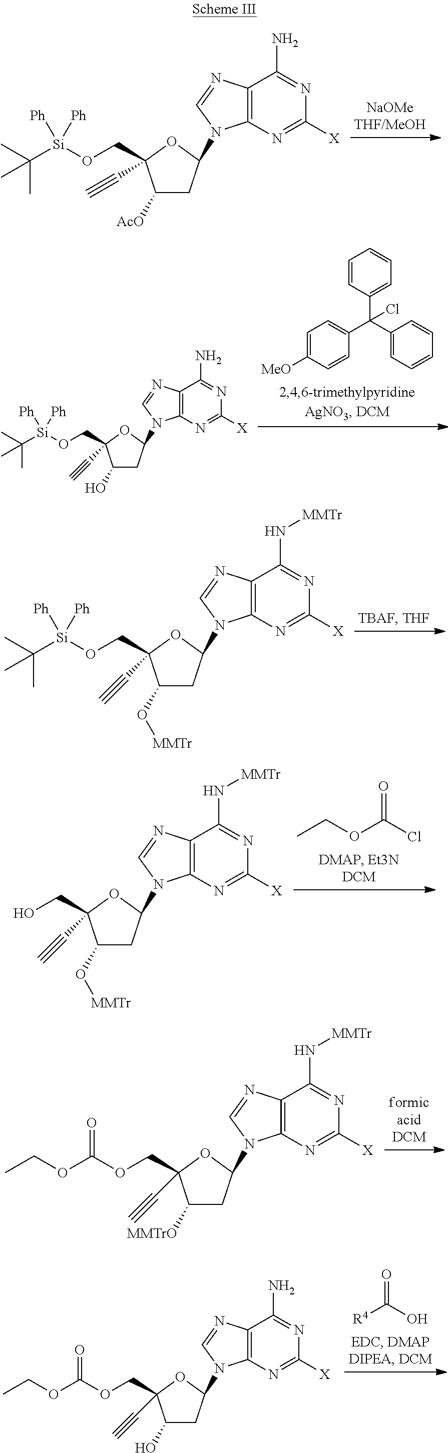

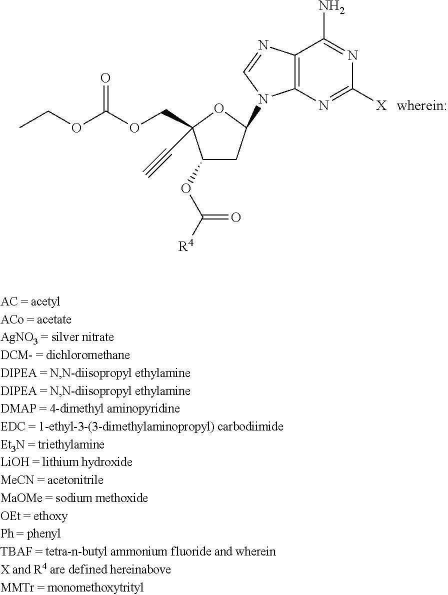

¹H NMR spectra were recorded on a Varian spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Waters (Acquity). The following conditions were employed described below.

### MS Conditions:

### Instrument: Waters SQD

### Serial Number: F06SQD018N

### Scan Mode: Alternating Positive/Negative Electrospray

### Scan Range: 125-1200 amu

Scan Time: 150 msec
InterScan Delay: 50 msec

### LC Conditions:

The UPLC analysis was conducted on a Phenomenex Kinetex 1.7 um
2.1×50 mm XB-C18 column at 40° C.
0.2 uL of sample was injected using PLNO (partial loop with needle overfill) injection mode.
The gradient employed was:

### Mobile Phase A: Water+0.2% v/v Formic Acid

### Mobile Phase B: Acetonitrile+0.15% v/v Formic Acid

| Time | % A | % B | Flow Rate |
|---|---|---|---|
| 0.00 min | 95 | 5 | 1 ml/min |
| 1.1 min | 1 | 99 | 1 ml/min |
| 1.5 min | 1 | 99 | 1 ml/min |

UV detection provided by summed absorbance signal from 210 to 350 nm scanning at 40 Hz.

### Example 1: (2R,3S,5R)-5-(6-amino-2-fluoro-9H-purin-9-yl)-2-ethynyl-2-(hydroxymethyl)tetrahydrofuran-3-yl acetate

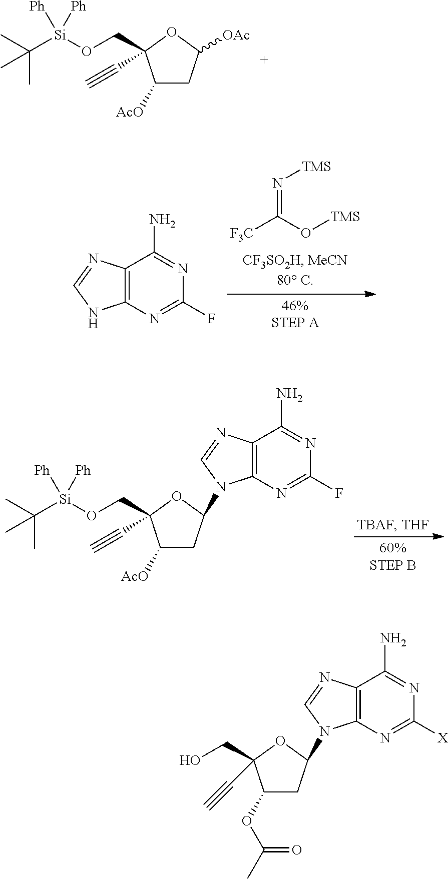

### Step A: (2R,3S,5R)-5-(6-amino-2-fluoro-9H-purin-9-yl)-2-(((tert-butyldiphenylsilyl)oxy)methyl)-2-ethynyltetrahydrofuran-3-yl acetate

A suspension of 2-fluoro-9H-purin-6-amine (0.545 g, 3.56 mmol) in anhydrous MeCN (10 mL) in a screw-capped glass pressure vessel under a nitrogen atmosphere was treated with trimethylsilyl 2,2,2-trifluoro-N-(trimethylsilyl)acetimidate (1.89 ml, 7.12 mmol) and heated to 80° C. with stirring in an oil bath. After 45 minutes most of the solid had dissolved. The solution was treated with (4S,5R)-5-(((tert-butyldiphenylsilyl)oxy)methyl)-5-ethynyltetrahydrofuran-2,4-diyl diacetate (1.14 g, 2.37 mmol, prepared according to Org. Lett., Vol. 13, No. 19, 2011) dissolved in MeCN (9 mL) followed by freshly prepared 0.2M trifluoromethanesulfonic acid/MeCN (2.37 ml, 0.474 mmol) (prepared by dissolving 44 μL of triflic acid in 2.5 mL of MeCN). The temperature was maintained at 80° C. After 1.5 hour at 80° C. LCMS indicated complete reaction. The solution was cooled to RT, quenched by addition of 1M aqueous HCl (3 mL). After stirring the mixture briefly, it was partitioned between saturated aqueous NaHCO₃ and EtOAc and the phases separated. The aqueous phase was extracted with EtOAc (2×). The combined EtOAc solutions were dried over Na₂SO₄ and concentrated at reduced pressure to give a tan solid. This material was subjected to flash chromatography (silica gel, 0-100% EtOAc/DCM) and the higher R_{f} component isolated to afford the title compound (0.63 g, 46%) as a white solid. LCMS (ESI) m/z calcd for C₃₀H₃₂FN₅O₄Si: 573.2. Found: 574.4 (M+1)⁺. ¹H NMR (400 MHz, METHANOL-d₄) δ 8.10 (s, 1H), 7.59-7.67 (m, 4H), 7.26-7.45 (m, 6H), 6.39 (t, J=6.6 Hz, 1H), 5.91 (dd, J=7.0, 5.5 Hz, 1H), 3.97 (d, J=10.9 Hz, 1H), 3.86 (d, J=10.9 Hz, 1H), 3.05-3.18 (m, 2H), 2.64-2.74 (m, 1H), 2.14 (s, 3H), 0.97-1.04 (m, 9H).

### Step B: (2R,3S,5R)-5-(6-amino-2-fluoro-9H-purin-9-yl)-2-ethynyl-2-(hydroxymethyl)tetrahydrofuran-3-yl acetate

To a solution of (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (29 mg, 0.051 mmol) in THF (0.5 mL) was added TBAF, 1M solution in THF (0.061 mL, 0.061 mmol) at ambient temperature and the mixture was allowed to stir for 45 minutes. The mixture was concentrated and then purified on silica gel (4 g column, 0-10% DCM/MeOH) to afford the title compound (10 mg, 60%) as a white solid. LCMS (ESI) m/z calcd for C₁₄H₁₄FN₅O₄: 335.1. Found: 336.2 (M+1)⁺. ¹H NMR (400 MHz, METHANOL-d₄) δ=8.26 (s, 1H), 6.46-6.40 (m, 1H), 5.70 (dd, J=3.1, 6.6 Hz, 1H), 3.89 (d, J=12.1 Hz, 1H), 3.82 (d, J=12.0 Hz, 1H), 3.16 (s, 1H), 3.05-2.96 (m, 1H), 2.65-2.58 (m, 1H), 2.15 (s, 3H).

### Example 2: (2R,3S,5R)-5-(6-amino-2-fluoro-9H-purin-9-yl)-2-ethynyl-2-(hydroxymethyl)tetrahydrofuran-3-yl tetradecanoate

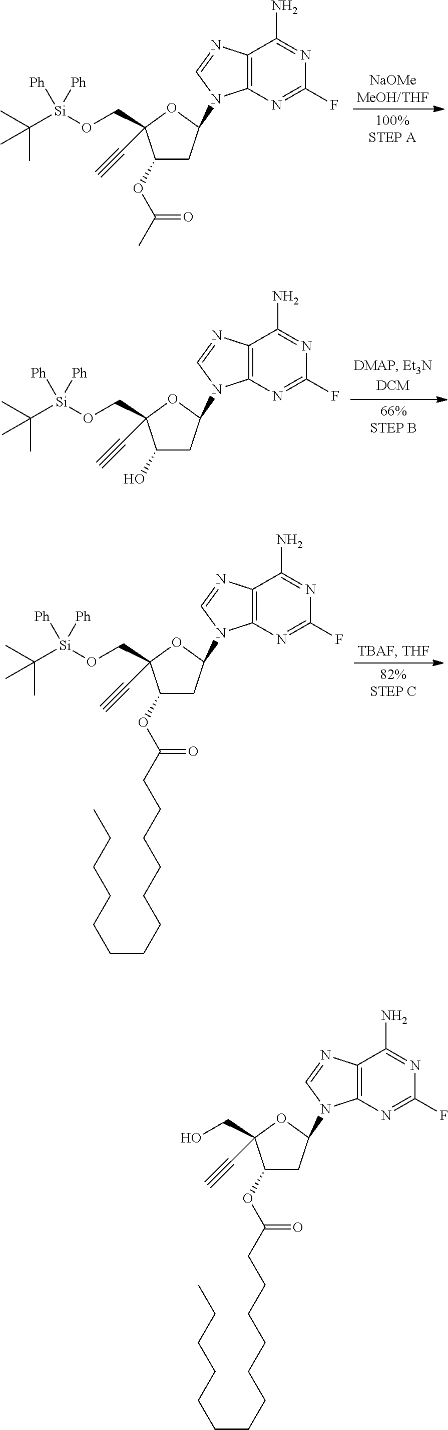 [SMILES: C#C[C@]1(CO[Si](c2ccccc2)(c2ccccc2)C(C)(C)C)O[C@@H](n2cnc3c(N)nc(F)nc32)C[C@@H]1OC(C)=O ; C#C[C@]1(CO[Si](c2ccccc2)(c2ccccc2)C(C)(C)C)O[C@@H](n2cnc3c(N)nc(F)nc32)C[C@@H]1O ; C#C[C@]1(CO[Si](c2ccccc2)(c2ccccc2)C(C)(C)C)O[C@@H](n2cnc3c(N)nc(F)nc32)C[C@@H]1OC(=O)CCCCCCCCCCCCC ; C#C[C@]1(CO)O[C@@H](n2cnc3c(N)nc(F)nc32)C[C@@H]1OC(=O)CCCCCCCCCCCCC]

### Step A: (2R,3S,5R)-5-(6-amino-2-fluoro-9H-purin-9-yl)-2-(((tert-butyldiphenylsilyl)oxy)methyl)-2-ethynyltetrahydrofuran-3-ol

To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (2.30 g, 4.01 mmol) in 1:1 THF/MeOH (8 mL) was added 25% NaOMe/MeOH (0.130 g, 0.601 mmol). The resulting solution was stirred at RT. After 30 minutes LCMS indicated complete reaction. The solution was treated with acetic acid (0.459 ml, 8.02 mmol) and concentrated to dryness at reduced pressure. The residue was partitioned between 8:2 chloroform/iPrOH and half-saturated aqueous NaHCO₃ and the phases separated. The aqueous phase was extracted with three additional portions of 8:2 chloroform/iPrOH. The combined organic solutions were dried over Na₂SO₄ and concentrated to dryness at reduced pressure to afford the title compound (2.13 g, 100%) as a white solid. LCMS (ESI) m/z calcd for C₂₈H₃₀FN₅O₃Si: 531.2. Found: 532.3 (M+1)⁺. ¹H NMR (400 MHz, DMSO-d₆) δ=8.27 (s, 1H), 7.97-7.73 (m, 2H), 7.60-7.56 (m, 2H), 7.55-7.50 (m, 2H), 7.46-7.38 (m, 2H), 7.37-7.28 (m, 4H), 6.27 (dd, J=3.5, 8.2 Hz, 1H), 5.73-5.69 (m, 1H), 4.88-4.79 (m, 1H), 3.86-3.72 (m, 2H), 3.56 (s, 1H), 3.34 (br s, 1H), 2.94-2.85 (m, 1H), 0.88 (s, 9H).

### Step B: (2R,3S,5R)-5-(6-amino-2-fluoro-9H-purin-9-yl)-2-(((tert-butyldiphenylsilyl)oxy)methyl)-2-ethynyltetrahydrofuran-3-yl tetradecanoate

To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-ol (31 mg, 0.058 mmol) and DMAP (7.12 mg, 0.058 mmol) in DCM (0.5 mL) at 0° C. was added triethylamine (0.024 mL, 0.175 mmol), followed by a solution of tetradecanoyl chloride (28.8 mg, 0.117 mmol) in DCM (0.2 mL). The mixture was warmed to ambient temperature and stirred overnight. The mixture was concentrated and then purified on silica gel (4 g column, 0-50% DCM/EtOAc) to afford the title compound (28.6 mg, 66%) as a colorless residue. LCMS (ESI) m/z calcd for C₄₂H₅₆FN₅O₄Si: 741.4. Found: 742.5 (M+1)⁺. ¹H NMR (400 MHz, CHLOROFORM-d) δ=7.97 (s, 1H), 7.71-7.64 (m, 4H), 7.48-7.34 (m, 6H), 6.50 (dd, J=6.2, 7.4 Hz, 1H), 5.93 (brs, 2H), 5.82 (dd, J=4.1, 6.8 Hz, 1H), 4.05 (d, J=10.9 Hz, 1H), 3.95 (d, J=11.3 Hz, 1H), 2.89-2.81 (m, 1H), 2.70-2.63 (m, 1H), 2.56 (s, 1H), 2.44-2.37 (m, 2H), 1.73-1.61 (m, 2H), 1.42-1.19 (m, 20H), 1.09 (s, 9H), 0.92-0.84 (m, 3H).

### Step C: (2R,3S,5R)-5-(6-amino-2-fluoro-9H-purin-9-yl)-2-ethynyl-2-(hydroxymethyl)tetrahydrofuran-3-yl tetradecanoate

To a solution of (2R,3S,5R)-5-(6-amino-2-fluoro-9H-purin-9-yl)-2-(((tert-butyldiphenylsilyl)oxy)methyl)-2-ethynyltetrahydrofuran-3-yl tetradecanoate (28.6 mg, 0.039 mmol) in THF (0.500 mL) was added TBAF, 1M solution in THF (0.070 mL, 0.070 mmol) at ambient temperature. The mixture was stirred for 20 minutes, treated with AcOH (6 drops) and then concentrated to dryness. The residue was purified on silica gel (4 g column, 0-10% DCM/MeOH) to afford the title compound (16.2 mg, 82%) as a colorless residue. LCMS (ESI) m/z calcd for C₂₆H₃₈FN₅O₄: 503.3. Found: 504.4 (M+1)⁺. ¹H NMR (400 MHz, DMSO-d₆) δ=8.34 (s, 1H), 8.06-7.77 (m, 2H), 6.31 (dd, J=6.4, 8.0 Hz, 1H), 5.62-5.53 (m, 2H), 3.72-3.66 (m, 1H), 3.64 (s, 1H), 3.63-3.56 (m, 1H), 3.04-2.94 (m, 1H), 2.54-2.48 (m, 1H), 2.41-2.35 (m, 2H), 1.62-1.53 (m, 2H), 1.34-1.15 (m, 20H), 0.89-0.80 (m, 3H).

### Example 3: (2R,3S,5R)-5-(6-amino-2-fluoro-9H-purin-9-yl)-2-ethynyl-2-(hydroxymethyl)tetrahydrofuran-3-yl decanoate

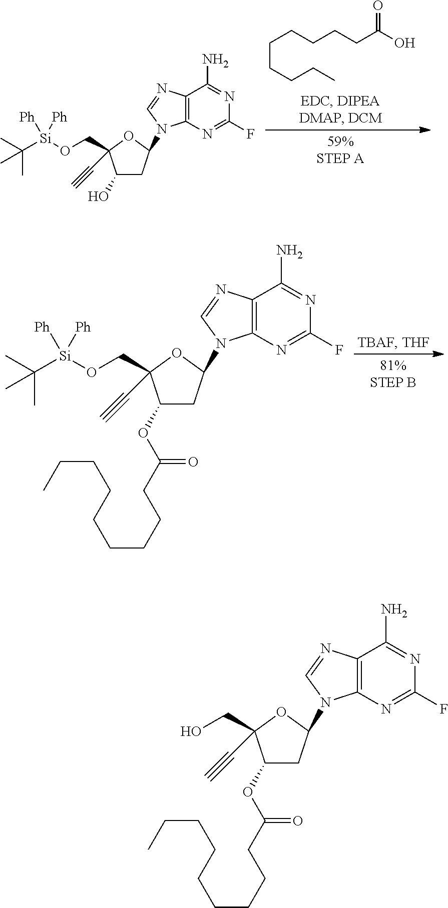 [SMILES: C#C[C@]1(CO[Si](c2ccccc2)(c2ccccc2)C(C)(C)C)O[C@@H](n2cnc3c(N)nc(F)nc32)C[C@@H]1O ; CCCCCCCCCC(=O)O ; C#C[C@]1(CO[Si](c2ccccc2)(c2ccccc2)C(C)(C)C)O[C@@H](n2cnc3c(N)nc(F)nc32)C[C@@H]1OC(=O)CCCCCCCCC ; C#C[C@]1(CO)O[C@@H](n2cnc3c(N)nc(F)nc32)C[C@@H]1OC(=O)CCCCCCCCC]

### Step A: (2R,3S,5R)-5-(6-amino-2-fluoro-9H-purin-9-yl)-2-(((tert-butyldiphenylsilyl)oxy)methyl)-2-ethynyltetrahydrofuran-3-yl decanoate

To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-ol (32.6 mg, 0.061 mmol), decanoic acid (21.13 mg, 0.123 mmol) and DMAP (7.49 mg, 0.061 mmol) in DCM (0.6 mL) was added EDC (35.3 mg, 0.184 mmol), followed by DIPEA (0.054 mL, 0.307 mmol) at ambient temperature and the mixture was allowed to stir overnight. The mixture was concentrated and then purified on silica gel (4 g column, 0-50% DCM/EtOAc) to afford the title compound (24.7 mg, 59%) as a colorless residue. LCMS (ESI) m/z calcd for C₃₈H₄₈FN₅O₄: 685.4. Found: 686.5 (M+1)⁺. ¹H NMR (400 MHz, CHLOROFORM-d) δ=7.97 (s, 1H), 7.71-7.64 (m, 4H), 7.48-7.34 (m, 6H), 6.50 (dd, J=6.2, 7.4 Hz, 1H), 5.91 (br s, 1H), 5.82 (dd, J=3.9, 7.0 Hz, 1H), 4.05 (d, J=10.9 Hz, 1H), 3.96 (d, J=11.3 Hz, 1H), 2.90-2.81 (m, 1H), 2.70-2.63 (m, 1H), 2.56 (s, 1H), 2.40-2.38 (m, 2H), 1.74-1.62 (m, 2H), 1.41-1.22 (m, 12H), 1.10 (s, 9H), 0.93-0.84 (m, 3H).

### Step B: (2R,3S,5R)-5-(6-amino-2-fluoro-9H-purin-9-yl)-2-ethynyl-2-(hydroxymethyl)tetrahydrofuran-3-yl decanoate

To a solution of (2R,3S,5R)-5-(6-amino-2-fluoro-9H-purin-9-yl)-2-(((tert-butyldiphenylsilyl)oxy)methyl)-2-ethynyltetrahydrofuran-3-yl decanoate (24.7 mg, 0.036 mmol) in THF (0.5 mL) at ambient temperature was added TBAF, 1M solution in THF (0.074 mL, 0.074 mmol). The mixture was stirred for 15 minutes, treated with AcOH (6 drops) and then concentrated to dryness. The residue was purified on silica gel (4 g column, 0-10% DCM/MeOH) to afford a colorless residue. LCMS (ESI) m/z calcd for C₂₂H₃₀FN₅O₄: 447.2. Found: 448.3 (M+1)⁺. ¹H NMR (400 MHz, DMSO-d₆) δ=8.34 (s, 1H), 8.10-7.73 (m, 2H), 6.31 (dd, J=6.4, 7.6 Hz, 1H), 5.64-5.50 (m, 2H), 3.75-3.54 (m, 3H), 3.05-2.95 (m, 1H), 2.57-2.44 (m, 1H, overlapping DMSO peak), 2.42-2.35 (m, 2H), 1.68-1.48 (m, 2H), 1.25 (m, 12H), 0.90-0.76 (m, 3H).

### Example 4: (2R,3S,5R)-5-(6-amino-2-fluoro-9H-purin-9-yl)-2-ethynyl-2-(hydroxymethyl)tetrahydrofuran-3-yl heptanoate

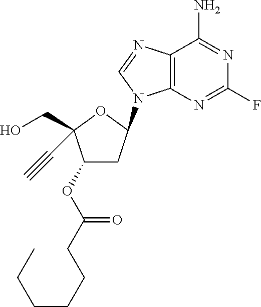 [SMILES: C#C[C@]1(CO)O[C@@H](n2cnc3c(N)nc(F)nc32)C[C@@H]1OC(=O)CCCCCC]

The title compound was made in a similar manner as Example 3 except using heptanoic acid in Step A and then purified using silica gel chromatography, followed by RP-HPLC (C18, 10-100% MeCN/water with 0.1% FA) in Step B. LCMS (ESI) m/z calcd for C₁₉H₂₄FN₅O₄: 405.2. Found: 406.3 (M+1)⁺. ¹H NMR (400 MHz, METHANOL-d₄) δ=8.27 (s, 1H), 6.46-6.40 (m, 1H), 5.71 (dd, J=3.5, 6.6 Hz, 1H), 3.89 (d, J=12.1 Hz, 1H), 3.83 (d, J=12.1 Hz, 1H), 3.16 (s, 1H), 3.05-2.96 (m, 1H), 2.65-2.57 (m, 1H), 2.47-2.41 (m, 2H), 1.73-1.63 (m, 2H), 1.45-1.24 (m, 6H), 0.96-0.86 (m, 3H).

### Example 5: (2R,3S,5R)-5-(6-amino-2-fluoro-9H-purin-9-yl)-2-ethynyl-2-(hydroxymethyl)tetrahydrofuran-3-yl 2-propylpentanoate

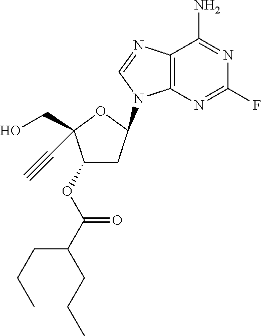 [SMILES: C#C[C@]1(CO)O[C@@H](n2cnc3c(N)nc(F)nc32)C[C@@H]1OC(=O)C(CCC)CCC]

The title compound was made in a similar manner as Example 3 except using 2-propylpentanoic acid in Step A. LCMS (ESI) m/z calcd for C₂₀H₂₆FN₅O₄: 419.2. Found: 420.3 (M+1)⁺. ¹H NMR (400 MHz, DMSO-d₆) δ=8.35 (s, 1H), 8.06-7.72 (m, 2H), 6.33-6.28 (m, 1H), 5.63-5.52 (m, 2H), 3.74-3.65 (m, 2H), 3.62-3.56 (m, 1H), 3.07-2.98 (m, 1H), 2.52-2.38 (m, 2H, overlapping DMSO peak), 1.66-1.53 (m, 2H), 1.50-1.39 (m, 2H), 1.36-1.20 (m, 4H), 0.91-0.85 (m, 6H).

### Example 6: (2R,3S,5R)-5-(6-amino-2-fluoro-9H-purin-9-yl)-2-ethynyl-2-(hydroxymethyl)tetrahydrofuran-3-yl icosanoate

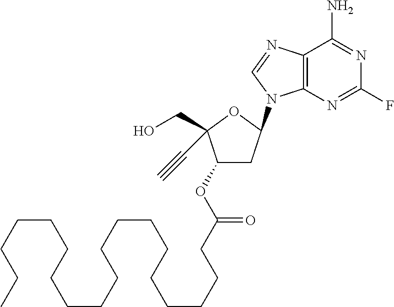 [SMILES: C#C[C@]1(CO)O[C@@H](n2cnc3c(N)nc(F)nc32)C[C@@H]1OC(=O)CCCCCCCCCCCCCCCCCCC]

The title compound was made in a similar manner as Example 3 except using icosanoic acid in Step A. LCMS (ESI) m/z calcd for C₃₂H₅₀FN₅O₄: 587.4. Found: 588.6 (M+1)⁺. ¹H NMR (400 MHz, DMSO-d₆) δ=8.34 (s, 1H), 8.06-7.76 (m, 2H), 6.31 (dd, J=6.2, 7.8 Hz, 1H), 5.62-5.54 (m, 2H), 3.72-3.66 (m, 1H), 3.65 (s, 1H), 3.63-3.57 (m, 1H), 3.06-2.90 (m, 1H), 2.54-2.47 (m, 1H, overlapping DMSO peak), 2.41-2.35 (m, 2H), 1.62-1.53 (m, 2H), 1.34-1.19 (m, 32H), 0.88-0.80 (m, 3H).

### Example 7: (9Z,12Z,15Z)-(2R,3S,5R)-5-(6-amino-2-fluoro-9H-purin-9-yl)-2-ethynyl-2-(hydroxymethyl)tetrahydrofuran-3-yl octadeca-9,12,15-trienoate

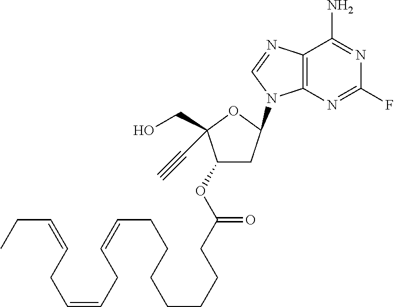 [SMILES: C#C[C@]1(CO)O[C@@H](n2cnc3c(N)nc(F)nc32)C[C@@H]1OC(=O)CCCCCCC/C=C\C/C=C\C/C=C\CC]

The title compound was made in a similar manner as Example 3 except using (9Z,12Z,15Z)-octadeca-9,12,15-trienoic acid in Step A. LCMS (ESI) m/z calcd for C₃₀H₄₀FN₅O₄: 553.3. Found: 554.3 (M+1)⁺. ¹H NMR (400 MHz, CHLOROFORM-d) δ=7.84 (s, 1H), 6.34 (dd, J=5.5, 9.4 Hz, 1H), 5.82-5.78 (m, 1H), 5.55 (dd, J=3.1, 11.3 Hz, 1H), 5.46-5.27 (m, 4H), 4.08-4.01 (m, 1H), 3.99-3.88 (m, 1H), 3.26-3.17 (m, 1H), 2.86-2.78 (m, 3H), 2.62 (s, 1H), 2.46-2.37 (m, 3H), 2.15-2.02 (m, 3H), 1.75-1.64 (m, 3H), 1.44-1.28 (m, 10H), 0.99 (t, J=7.6 Hz, 3H).

### Example 8: (2R,3S,5R)-5-(6-((ethoxycarbonyl)amino)-2-fluoro-9H-purin-9-yl)-2-ethynyl-2-(hydroxymethyl)tetrahydrofuran-3-yl decanoate

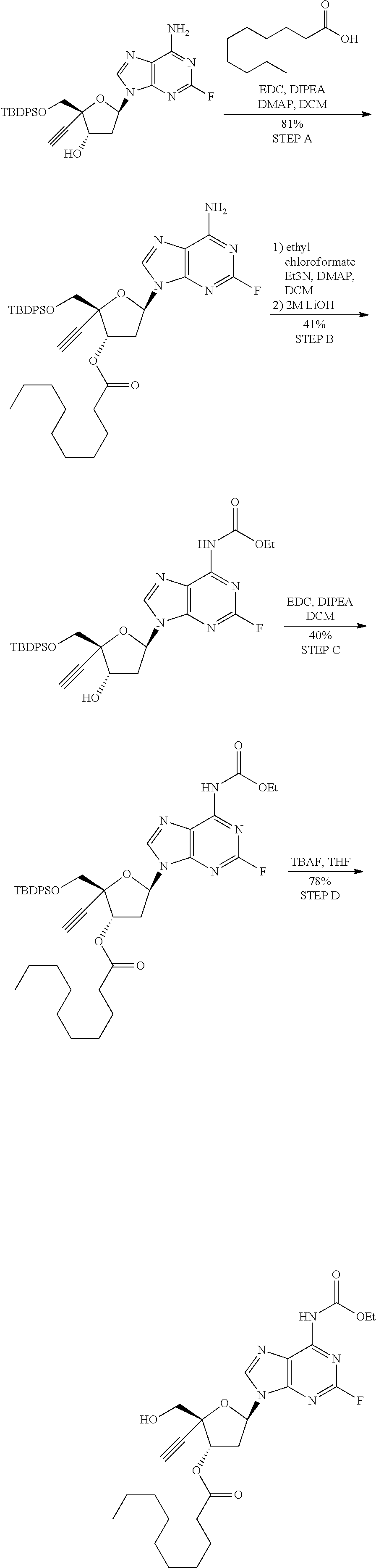

### Step A: (2R,3S,5R)-5-(6-amino-2-fluoro-9H-purin-9-yl)-2-(((tert-butyldiphenylsilyl)oxy)methyl)-2-ethynyltetrahydrofuran-3-yl decanoate

To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-ol (40.3 mg, 0.076 mmol), decanoic acid (26.1 mg, 0.152 mmol) and DMAP (9.26 mg, 0.076 mmol) in DCM (0.6 mL) was added EDC (43.6 mg, 0.227 mmol), followed by DIPEA (0.066 mL, 0.379 mmol) at ambient temperature and the mixture was stirred for 130 minutes. The mixture was concentrated to dryness and then purified on silica gel (4 g column, 0-50% DCM/EtOAc) to afford a colorless residue (42 mg, 81%). LCMS (ESI) m/z calcd for C₃₈H₄₈FN₅O₄Si: 685.4. Found: 686.4 (M+1)⁺.

### Step B: ethyl (9-((2R,4S,5R)-5-(((tert-butyldiphenylsilyl)oxy)methyl)-5-ethynyl-4-hydroxytetrahydrofuran-2-yl)-2-fluoro-9H-purin-6-yl)carbamate

To a solution of (2R,3S,5R)-5-(6-amino-2-fluoro-9H-purin-9-yl)-2-(((tert-butyldiphenylsilyl)oxy)methyl)-2-ethynyltetrahydrofuran-3-yl decanoate (42 mg, 0.061 mmol) and DMAP (7.45 mg, 0.061 mmol) in DCM (0.5 mL) at 0° C. was added triethylamine (0.026 mL, 0.183 mmol) followed by a solution of ethyl chloroformate (0.015 mL, 0.153 mmol) in DCM (85 uL). The mixture was stirred at 0° C. for 5 minutes, warmed to ambient temperature and then stirred for one hour. Saturated NaHCO₃ was added and the mixture was extracted with EtOAc. The extracts were washed with brine, dried over Na₂SO₄, filtered and concentrated to a yellow residue. The residue was dissolved in a mixture of THF (0.600 mL) and MeOH (0.2 mL) and then treated with 2M LiOH (0.061 mL, 0.122 mmol) at ambient temperature. The mixture was allowed to stir for 10 minutes. 1N HCl (120 uL) was added, the mixture was diluted with water and then extracted with EtOAc (brine was added to help clear emulsion). The extracts were washed with brine, dried over Na₂S04, filtered and concentrated. The residue was purified on silica gel (4 g column, 0-50% DCM/EtOAc) to afford the title compound (15.2 mg, 41%) as a colorless residue. LCMS (ESI) m/z calcd for C₃₁H₃₄FN₅O₅Si: 603.2. Found: 604.3 (M+1)⁺.

### Step C: (2R,3S,5R)-2-(((tert-butyldiphenylsilyl)oxy)methyl)-5-(6-((ethoxycarbonyl)amino)-2-fluoro-9H-purin-9-yl)-2-ethynyltetrahydrofuran-3-yl decanoate

To a stirred solution of ethyl (9-((2R,4S,5R)-5-(((tert-butyldiphenylsilyl)oxy)methyl)-5-ethynyl-4-hydroxytetrahydrofuran-2-yl)-2-fluoro-9H-purin-6-yl)carbamate (15.2 mg, 0.025 mmol), decanoic acid (8.61 mg, 0.05 mmol) and DMAP (3.05 mg, 0.025 mmol) in DCM (0.4 mL) at ambient temperature was added EDC (14.38 mg, 0.075 mmol) and DIPEA (0.022 mL, 0.125 mmol). The mixture was stirred for two hours, concentrated and then purified on silica gel (4 g column, 0-50% DCM/EtOAc) to afford a colorless residue (7.5 mg, 40%). LCMS (ESI) m/z calcd for C₄₁H₅₂FN₅O₆Si: 757.4. Found: 758.4 (M+1)⁺.

### Step D: (2R,3S,5R)-5-(6-((ethoxycarbonyl)amino)-2-fluoro-9H-purin-9-yl)-2-ethynyl-2-(hydroxymethyl)tetrahydrofuran-3-yl decanoate

To a solution of (2R,3S,5R)-2-(((tert-butyldiphenylsilyl)oxy)methyl)-5-(6-((ethoxycarbonyl)amino)-2-fluoro-9H-purin-9-yl)-2-ethynyltetrahydrofuran-3-yl decanoate (7.5 mg, 0.01 mmol) in THF (0.4 mL) at ambient temperature was added TBAF, 1M solution in THF (0.015 mL, 0.015 mmol) and the mixture was stirred for 5 minutes. The mixture was concentrated, then purified by RP-HPLC (C18, 10-100% MeCN/water with 0.1% FA) to afford the title compound (4 mg, 78%) as a colorless residue. LCMS (ESI) m/z calcd for C25H₃₄FN₅O₆: 519.3. Found: 520.8 (M+1)⁺. ¹H NMR (400 MHz, DMSO-d₆) δ=11.11-10.85 (m, 1H), 8.61 (s, 1H), 6.42-6.37 (m, 1H), 5.61 (dd, J=3.7, 6.4 Hz, 1H), 5.56-5.51 (m, 1H), 4.18 (q, J=7.3 Hz, 2H), 3.73-3.55 (m, 3H), 3.10-3.01 (m, 1H), 2.61-2.53 (m, 1H), 2.42-2.35 (m, 2H), 1.62-1.53 (m, 2H), 1.35-1.19 (m, 15H), 0.88-0.81 (m, 3H).

### Example 9: (2R,3S,5R)-5-(6-amino-2-fluoro-9H-purin-9-yl)-2-(((ethoxycarbonyl)oxy)methyl)-2-ethynyltetrahydrofuran-3-yl decanoate

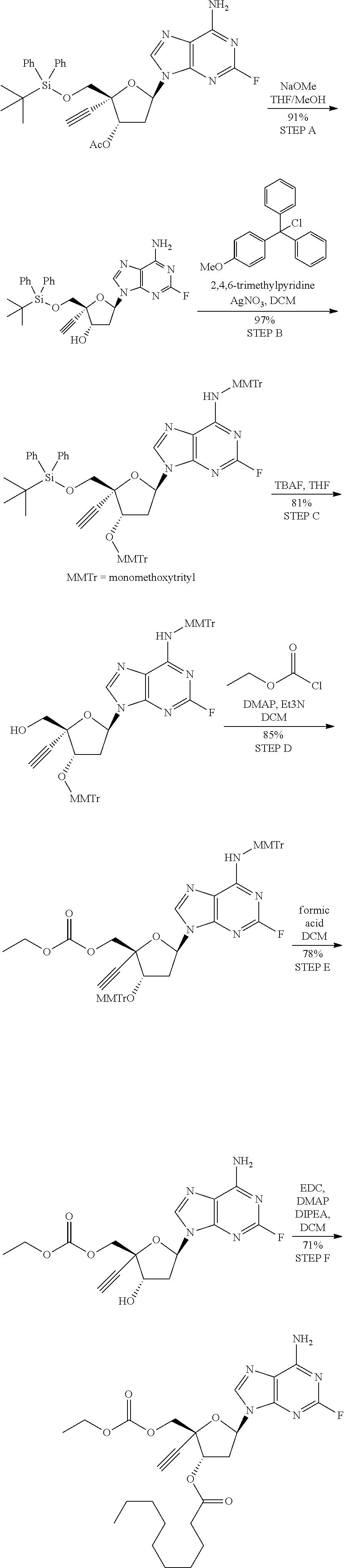

### Step A: (2R,3S,5R)-5-(6-amino-2-fluoro-9H-purin-9-yl)-2-(((tert-butyldiphenylsilyl)oxy)methyl)-2-ethynyltetrahydrofuran-3-ol

To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (0.62 g, 1.08 mmol) in 1:1 THF/MeOH (4 mL) was added 25% NaOMe/MeOH (3 drops). The resulting solution was stirred at RT. After 30 minutes LCMS indicated complete reaction. The solution was treated with glacial AcOH (5 drops) and concentrated to dryness at reduced pressure. The residue was partitioned between 8:2 chloroform/iPrOH and half-saturated aqueous NaHCO₃ and the phases separated. The aqueous phase was extracted with two additional portions of 8:2 chloroform/iPrOH. The combined organic solutions were dried over Na₂SO₄ and concentrated to dryness at reduced pressure to afford the title compound (0.52 g, 91%) as a white solid. LCMS (ESI) m/z calcd for C₂₈H₃₀FN₅O₃Si: 531.2. Found: 532.3 (M+1)⁺. ¹H NMR (400 MHz, METHANOL-d₄) δ 8.17 (s, 1H), 7.53-7.66 (m, 4H), 7.22-7.45 (m, 6H), 6.32 (dd, J=7.8, 3.1 Hz, 1H), 5.01 (t, J=7.8 Hz, 1H), 3.87 (q, J=11.3 Hz, 2H), 3.05 (s, 1H), 2.90-2.99 (m, 1H), 2.63-2.72 (m, 1H), 0.94 (s, 9H).

### Step B: 9-((2R,4S,5R)-5-(((tert-butyldiphenylsilyl)oxy)methyl)-5-ethynyl-4-((4-methoxyphenyl)diphenylmethoxy)tetrahydrofuran-2-yl)-2-fluoro-N-((4-methoxyphenyl)diphenylmethyl)-9H-purin-6-amine

To a stirred suspension of (2R,3S,5R)-5-(6-amino-2-fluoro-9H-purin-9-yl)-2-(((tert-butyldiphenylsilyl)oxy)methyl)-2-ethynyltetrahydrofuran-3-ol (0.510 g, 0.959 mmol) in DCM (8 mL) was added silver nitrate (0.489 g, 2.88 mmol), 2,4,6-trimethylpyridine (0.766 ml, 5.76 mmol), and (chloro(4-methoxyphenyl)methylene)dibenzene (0.889 g, 2.88 mmol). The resulting orange suspension was stirred at RT. After 2 hours LCMS indicated complete reaction. The mixture was diluted with EtOAc and filtered through celite to remove solids. The filtrate was washed with 10% aqueous citric acid (2×), saturated aqueous NaHCO₃ (2×), dried over Na₂SO₄ and concentrated at reduced pressure to give a pale yellow foam. This material was subjected to flash chromatography (silica gel, 0-100% EtOAc/hexanes) to afford the title compound (1.00 g, 97%) as a white foam. LCMS (ESI) m/z calcd for C₆₈H₆₂FN₅O₅Si: 1075.5. Found: 1076.7 (M+1)⁺. ¹H NMR (400 MHz, CDCl₃) δ 7.12-7.62 (m, 35H), 6.98 (s, 1H), 6.74-6.82 (m, 4H), 6.22 (t, J=6.6 Hz, 1H), 4.75 (t, J=5.9 Hz, 1H), 3.93 (d, J=11.3 Hz, 1H), 3.86 (d, J=11.3 Hz, 1H), 3.77 (s, 3H), 3.75 (s, 3H), 2.77 (s, 1H), 1.71 (t, J=6.3 Hz, 2H), 0.87 (s, 9H).

### Step C: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol

To a stirred solution of 9-((2R,4S,5R)-5-(((tert-butyldiphenylsilyl)oxy)methyl)-5-ethynyl-4-((4-methoxyphenyl)diphenylmethoxy)tetrahydrofuran-2-yl)-2-fluoro-N-((4-methoxyphenyl)diphenylmethyl)-9H-purin-6-amine (0.99 g, 0.92 mmol) in THF (8 mL) was added 1M TBAF/THF (1.38 ml, 1.38 mmol) by dropwise addition. The resulting solution was stirred at RT. After 1 hour LCMS indicated complete reaction. The solution was treated with glacial AcOH (0.10 mL) and concentrated at reduced pressure. The residue was dissolved in MeOH/DCM and again concentrated to dryness. The residue was subjected to flash chromatography (silica gel, 0-100% EtOAc/hexanes) to afford the title compound (0.623 g, 81%) as a white solid. LCMS (ESI) m/z calcd for C₅₂H₄₄FN₅O₅: 837.3. Found: 838.6 (M+1)⁺. ¹H NMR (400 MHz, DMSO-d₆) δ 8.04 (s, 1H), 8.00 (s, 1H), 7.47-7.54 m, 4H), 7.12-7.38 (m, 20H), 6.79-6.88 (m, 4H), 6.04 (t, J=6.3 Hz, 1H), 5.15 (t, J=6.1 Hz, 1H), 4.47 (t, J=6.1 Hz, 1H), 3.84 (s, 1H), 3.69 (s, 3H), 3.67 (s, 3H), 3.49-3.57 (m, 1H), 3.38-3.47 (m, 1H), 1.63-1.72 (m, 1H), 1.49-1.58 (m, 1H).

### Step D: ethyl (((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl) carbonate

To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (49 mg, 0.058 mmol) and DMAP (7.14 mg, 0.058 mmol) in DCM (0.7 mL) at 0° C. was added triethylamine (0.024 mL, 0.175 mmol), followed by a solution of ethyl carbonochloridate (0.011 mL, 0.117 mmol) in DCM (0.1 mL). The mixture was stirred at 0° C. for 5 minutes and then warmed to ambient temperature and stirred for 90 minutes. The mixture was concentrated and then purified on silica gel (4 g column, 0-50% DCM/EtOAc) to afford the title compound (45 mg, 85%) as a white solid. LCMS (ESI) m/z calcd for C55H₄₈FN₅O₇: 909.4. Found: 910.4 (M+1)⁺.

### Step E: ((2R,3S,5R)-5-(6-amino-2-fluoro-9H-purin-9-yl)-2-ethynyl-3-hydroxytetrahydrofuran-2-yl)methyl ethyl carbonate

To a solution of ethyl (((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl) carbonate (45 mg, 0.049 mmol) in DCM (1.0 mL) was added dropwise formic acid (0.5 mL, 13.04 mmol) and the mixture was allowed to stir at ambient temperature for 30 minutes. The mixture was concentrated and then purified on silica gel (4 g column, 0-10% DCM/MeOH) to afford the title compound (13.8 mg, 78%) as a white solid. LCMS (ESI) m/z calcd for C₁₅H₁₆FN₅O₅: 365.1. Found: 366.2 (M+1)⁺. ¹H NMR (400 MHz, METHANOL-d₄) δ=8.15 (s, 1H), 6.33 (dd, J=3.9, 7.8 Hz, 1H), 4.87-4.81 (m, 1H), 4.51 (d, J=11.7 Hz, 1H), 4.31 (d, J=12.1 Hz, 1H), 4.15-3.99 (m, 2H), 3.20 (s, 1H), 2.90-2.83 (m, 1H), 2.71-2.62 (m, 1H), 1.24-1.18 (m, 3H).

### Step F: (2R,3S,5R)-5-(6-amino-2-fluoro-9H-purin-9-yl)-2-(((ethoxycarbonyl)oxy)methyl)-2-ethynyltetrahydrofuran-3-yl decanoate

To a stirred solution of ((2R,3S,5R)-5-(6-amino-2-fluoro-9H-purin-9-yl)-2-ethynyl-3-hydroxytetrahydrofuran-2-yl)methyl ethyl carbonate (13.8 mg, 0.038 mmol) and DMAP (7.14 mg, 0.058 mmol) in DCM (0.5 mL) was added EDC (21.85 mg, 0.114 mmol) and DIPEA (0.033 mL, 0.19 mmol) at ambient temperature and the mixture was allowed to stir for approximately 3 hours. The mixture was concentrated and then purified on silica gel (0-50% DCM/EtOAc) to afford the title compound (14 mg, 71%) as a white solid. LCMS (ESI) m/z calcd for C₂₅H₃₄FN₅O₆: 519.3. Found: 520.3 (M+1)⁺. ¹H NMR (400 MHz, DMSO-d₆) δ=8.31 (s, 1H), 8.07-7.74 (m, 2H), 6.38-6.32 (m, 1H), 5.69 (dd, J=5.3, 6.8 Hz, 1H), 4.46 (d, J=11.3 Hz, 1H), 4.29 (d, J=11.3 Hz, 1H), 4.13-4.00 (m, 2H), 3.81 (s, 1H), 3.18-3.07 (m, 1H), 2.71-2.57 (m, 1H), 2.44-2.34 (m, 2H), 1.65-1.51 (m, 2H), 1.36-1.20 (m, 12H), 1.17 (t, J=7.0 Hz, 3H), 0.90-0.82 (m, 3H). 82 (m, 3H).

### Example 10: (2R,3S,5R)-5-(6-amino-2-fluoro-9H-purin-9-yl)-2-ethynyl-2-(hydroxymethyl)tetrahydrofuran-3-yl 2-phenylacetate

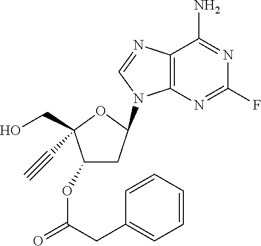 [SMILES: C#C[C@]1(CO)O[C@@H](n2cnc3c(N)nc(F)nc32)C[C@@H]1OC(=O)Cc1ccccc1]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting phenylacetic acid for 2-hexyldecanoic acid in step A. LCMS (ESI) m/z calcd for C₂₀H₁₈FN₅O₄: 411.1. Found: 412.3 (M+1)⁺. ¹H NMR (400 MHz, METHANOL-d₄) δ 8.25 (s, 1H), 7.44-7.17 (m, 5H), 6.41 (dd, J=6.2, 7.6 Hz, 1H), 5.71 (dd, J=3.6, 6.7 Hz, 1H), 3.91-3.70 (m, 4H), 3.10-2.90 (m, 2H), 2.60 (ddd, J=3.5, 6.2, 13.9 Hz, 1H).

### Example 11: (2R,3S,5R)-5-(6-amino-2-fluoro-9H-purin-9-yl)-2-ethynyl-2-(hydroxymethyl)tetrahydrofuran-3-yl 2-methylheptanoate

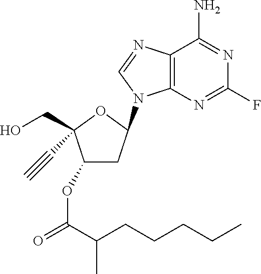 [SMILES: C#C[C@]1(CO)O[C@@H](n2cnc3c(N)nc(F)nc32)C[C@@H]1OC(=O)C(C)CCCCC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting 2-methylheptanoic acid for 2-hexyldecanoic acid in step A. LCMS (ESI) m/z calcd for C₂₀H₂₆FN₅O₄: 419.2. Found: 420.3 (M+1)⁺. ¹H NMR (400 MHz, CDCl₃) δ 7.88-7.84 (m, 1H), 6.34 (dd, J=5.7, 9.1 Hz, 1H), 5.98 (br s, 2H), 5.77 (dd, J=2.1, 6.2 Hz, 1H), 5.43-5.30 (m, 1H), 4.11-3.87 (m, 2H), 3.25-3.14 (m, 1H), 2.64-2.59 (m, 1H), 2.59-2.42 (m, 2H), 1.84-1.69 (m, 1H), 1.53-1.42 (m, 1H), 1.41-1.26 (m, 6H), 1.26-1.19 (m, 3H), 0.95-0.86 (m, 3H).

### Example 12: (2R,3S,5R)-5-(6-amino-2-fluoro-9H-purin-9-yl)-2-ethynyl-2-(hydroxymethyl)tetrahydrofuran-3-yl (1s,4S)-4-pentylcyclohexane-1-carboxylate

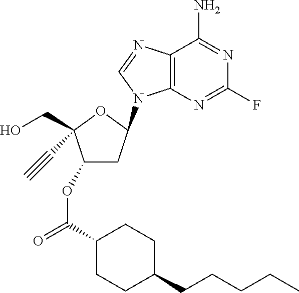 [SMILES: C#C[C@]1(CO)O[C@@H](n2cnc3c(N)nc(F)nc32)C[C@@H]1OC(=O)[C@H]1CC[C@H](CCCCC)CC1]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting trans-4-pentylcyclohexane-1-carboxylic acid for 2-hexyldecanoic acid in step A. LCMS (ESI) m/z calcd for C₂₄H₃₂FN₅O₄: 473.2. Found: 474.3 (M+1)⁺. ¹H NMR (400 MHz, CDCl₃) δ 7.85 (s, 1H), 6.34 (dd, J=5.6, 9.2 Hz, 1H), 5.96 (brs, 2H), 5.76 (dd, J=1.7, 6.2 Hz, 1H), 5.40 (br dd, J=2.9, 11.0 Hz, 1H), 4.08-3.88 (m, 2H), 3.19 (ddd, J=6.3, 9.2, 13.8 Hz, 1H), 2.62 (s, 1H), 2.45 (ddd, J=1.9, 5.6, 13.7 Hz, 1H), 2.39-2.27 (m, 1H), 2.13-1.99 (m, 2H), 1.90-1.81 (m, 2H), 1.49 (tq, J=3.3, 12.8 Hz, 2H), 1.37-1.17 (m, 9H), 1.02-0.92 (m, 2H), 0.92-0.86 (m, 3H).

### Example 13: (2R,3S,5R)-5-(6-amino-2-fluoro-9H-purin-9-yl)-2-ethynyl-2-(hydroxymethyl)tetrahydrofuran-3-yl pivalate

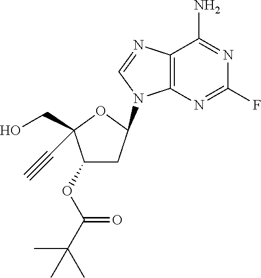 [SMILES: C#C[C@]1(CO)O[C@@H](n2cnc3c(N)nc(F)nc32)C[C@@H]1OC(=O)C(C)(C)C]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting pivalic acid for 2-hexyldecanoic acid in step A. LCMS (ESI) m/z calcd for C₁₇H₂₀FN₅O₄: 377.2. Found: 378.5 (M+1)⁺. ¹H NMR (400 MHz, DMSO-d₆) δ 8.35 (s, 1H), 8.09-7.77 (m, 2H), 6.31 (dd, J=6.2, 7.8 Hz, 1H), 5.63-5.57 (m, 1H), 5.54 (dd, J=3.1, 6.2 Hz, 1H), 3.74-3.66 (m, 2H), 3.64-3.55 (m, 1H), 3.19-3.13 (m, 1H), 3.08-2.95 (m, 1H), 1.22 (s, 9H).

### Example 14: (2R,3S,5R)-2-ethynyl-5-(2-fluoro-6-tetradecanamido-9H-purin-9-yl)-2-(hydroxymethyl)tetrahydrofuran-3-yl acetate

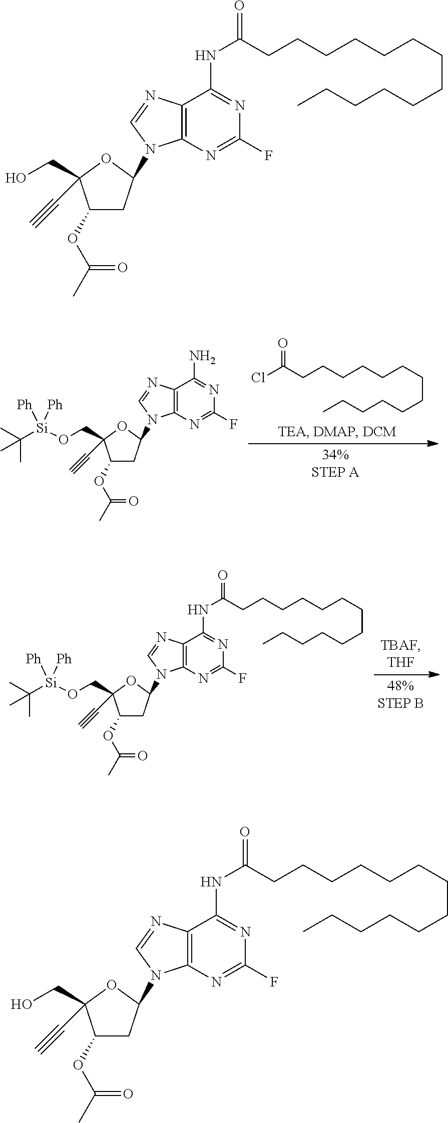 [SMILES: C#C[C@]1(CO)O[C@@H](n2cnc3c(NC(=O)CCCCCCCCCCCCC)nc(F)nc32)C[C@@H]1OC(C)=O ; C#C[C@]1(CO[Si](c2ccccc2)(c2ccccc2)C(C)(C)C)O[C@@H](n2cnc3c(N)nc(F)nc32)C[C@@H]1OC(C)=O ; CCCCCCCCCCCCCC(=O)Cl ; C#C[C@]1(CO[Si](c2ccccc2)(c2ccccc2)C(C)(C)C)O[C@@H](n2cnc3c(NC(=O)CCCCCCCCCCCCC)nc(F)nc32)C[C@@H]1OC(C)=O ; C#C[C@]1(CO)O[C@@H](n2cnc3c(NC(=O)CCCCCCCCCCCCC)nc(F)nc32)C[C@@H]1OC(C)=O]

### Step A: (2R,3S,5R)-2-(((tert-butyldiphenylsilyl)oxy)methyl)-2-ethynyl-5-(2-fluoro-6-tetradecanamido-9H-purin-9-yl)tetrahydrofuran-3-yl acetate

An ice cold solution of (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (200 mg, 0.349 mmol) and DMAP (42.6 mg, 0.349 mmol) in DCM (3.5 mL) was treated with TEA (0.146 mL, 1.05 mmol), followed by a solution of tetradecanoyl chloride (0.142 mL, 0.523 mmol) in DCM (1 mL). The reaction was stirred at 0° C. for 10 min, and then at RT for 18 h. The reaction mixture was concentrated to to dryness at reduced pressure. The residue was subjected to flash chromatography (silica gel, 0-30% EtOAc/DCM) to afford the title compound (93 mg, 34%) as clear film. LCMS (ESI) m/z calcd for C₄₄H₅₈FN₅O₅Si: 783.4. Found: 784.7 (M+1)⁺. ¹H NMR (400 MHz, CDCl₃) δ 8.51 (s, 1H), 8.09 (s, 1H), 7.72-7.59 (m, 4H), 7.51-7.33 (m, 6H), 6.52 (t, J=6.6 Hz, 1H), 5.82 (dd, J=3.9, 7.0 Hz, 1H), 4.12-3.89 (m, 2H), 3.00-2.80 (m, 3H), 2.77-2.64 (m, 1H), 2.60 (s, 1H), 2.17 (s, 3H), 1.76 (quin, J=7.5 Hz, 2H), 1.46-1.17 (m, 20H), 1.09 (s, 9H), 0.89 (t, J=6.8 Hz, 3H).

### Step B: (2R,3S,5R)-2-ethynyl-5-(2-fluoro-6-tetradecanamido-9H-purin-9-yl)-2-(hydroxymethyl)tetrahydrofuran-3-yl acetate

A solution of (2R,3S,5R)-2-(((tert-butyldiphenylsilyl)oxy)methyl)-2-ethynyl-5-(2-fluoro-6-tetradecanamido-9H-purin-9-yl)tetrahydrofuran-3-yl acetate (177 mg, 0.226 mmol) in THF (2.2 mL) was treated with TBAF (1M in THF) (0.293 mL, 0.293 mmol) and stirred at RT for 85 min. The reaction mixture was concentrated to dryness and the residue subjected to flash chromatography (silica gel, 0-100% EtOAc/DCM, then 0-20% MeOH/EtOAc) to give the title compound (62 mg, 48%) as a white solid. LCMS (ESI) m/z calcd for C₂₈H₄₀FN₅O₅: 545.3. Found: 546.5 (M+1)⁺. ¹H NMR (400 MHz, CDCl₃) δ 8.51 (s, 1H), 8.02 (s, 1H), 6.40 (dd, J=5.5, 9.0 Hz, 1H), 5.80 (dd, J=2.0, 6.2 Hz, 1H), 4.86 (dd, J=3.7, 11.1 Hz, 1H), 4.12-3.89 (m, 2H), 3.17 (ddd, J=6.4, 9.0, 13.9 Hz, 1H), 2.96 (t, J=7.6 Hz, 2H), 2.66 (s, 1H), 2.53 (ddd, J=2.0, 5.7, 13.9 Hz, 1H), 2.19 (s, 3H), 1.76 (quin, J=7.4 Hz, 2H), 1.49-1.15 (m, 20H), 0.89 (t, J=6.8 Hz, 3H).

### Example 15: (2R,3S,5R)-5-(6-amino-2-fluoro-9H-purin-9-yl)-2-ethynyl-2-(hydroxymethyl)tetrahydrofuran-3-yl 2-hexyldecanoate

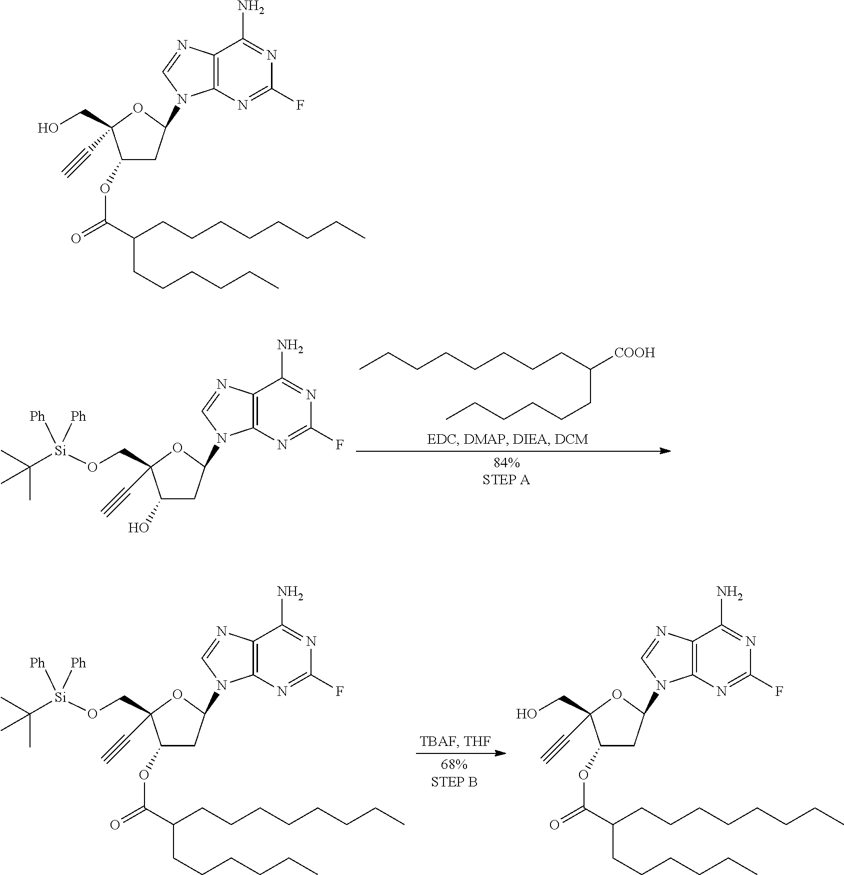 [SMILES: C#C[C@]1(CO)O[C@@H](n2cnc3c(N)nc(F)nc32)C[C@@H]1OC(=O)C(CCCCCC)CCCCCCCC ; C#C[C@]1(CO[Si](c2ccccc2)(c2ccccc2)C(C)(C)C)O[C@@H](n2cnc3c(N)nc(F)nc32)C[C@@H]1O ; CCCCCCCCC(CCCCCC)C(=O)O ; C#C[C@]1(CO[Si](c2ccccc2)(c2ccccc2)C(C)(C)C)O[C@@H](n2cnc3c(N)nc(F)nc32)C[C@@H]1OC(=O)C(CCCCCC)CCCCCCCC ; C#C[C@]1(CO)O[C@@H](n2cnc3c(N)nc(F)nc32)C[C@@H]1OC(=O)C(CCCCCC)CCCCCCCC]

### Step A: (2R,3S,5R)-5-(6-amino-2-fluoro-9H-purin-9-yl)-2-(((tert-butyldiphenylsilyl)oxy)methyl)-2-ethynyltetrahydrofuran-3-yl 2-hexyldecanoate

A suspension of (2R,3S,5R)-5-(6-amino-2-fluoro-9H-purin-9-yl)-2-(((tert-butyldiphenylsilyl)oxy)methyl)-2-ethynyltetrahydrofuran-3-ol (50 mg, 0.094 mmol) in DCM (1.5 mL) was treated with 2-hexyldecanoic acid (0.055 mL, 0.19 mmol), DMAP (11.5 mg, 0.094 mmol), EDC (54.1 mg, 0.282 mmol), DIEA (0.082 mL, 0.47 mmol), and stirred at RT for 18 h. The reaction was concentrated and the residue purified by flash chromatography (silica gel, 0-75% EtOAc/hexanes) to give mix (2R,3S,5R)-5-(6-amino-2-fluoro-9H-purin-9-yl)-2-(((tert-butyldiphenylsilyl)oxy)methyl)-2-ethynyltetrahydrofuran-3-yl 2-hexyldecanoate (61 mg, 84%) as clear film. LCMS (ESI) m/z calcd for C₄₄H₆₀FN₅O₄Si: 769.4. Found: 770.7 (M+1)⁺.

### Step B: (2R,3S,5R)-5-(6-amino-2-fluoro-9H-purin-9-yl)-2-ethynyl-2-(hydroxymethyl)tetrahydrofuran-3-yl 2-hexyldecanoate

An ice cold solution of mix (2R,3S,5R)-5-(6-amino-2-fluoro-9H-purin-9-yl)-2-(((tert-butyldiphenylsilyl)oxy)methyl)-2-ethynyltetrahydrofuran-3-yl 2-hexyldecanoate (61 mg, 0.079 mmol) in THF (1.6 mL) was treated with TBAF (1M in THF) (0.119 mL, 0.118 mmol) and stirred at 0° C. for 2 h. The reaction was quenched with AcOH (˜0.5 mL), diluted with water, and extracted with EtOAc. The combined organics were washed with brine (5×), dried over Na₂SO₄, filtered, and concentrated. Purification by flash chromatography (silica gel, 0-100% EtOAc/DCM) afforded (2R,3S,5R)-5-(6-amino-2-fluoro-9H-purin-9-yl)-2-ethynyl-2-(hydroxymethyl)tetrahydrofuran-3-yl 2-hexyldecanoate (29.4 mg, 68%) as white solid. LCMS (ESI) m/z calcd for C₂₈H₄₂FN₅O₄: 531.3. Found: 532.5 (M+1)⁺. ¹H NMR (400 MHz, CDCl₃) δ 7.86 (s, 1H), 6.33 (dd, J=5.7, 8.6 Hz, 1H), 6.12-5.83 (m, 2H), 5.77 (dd, J=2.5, 6.3 Hz, 1H), 5.29 (dd, J=3.7, 11.1 Hz, 1H), 4.13-4.00 (m, 1H), 3.98-3.85 (m, 1H), 3.18 (ddd, J=6.4, 8.6, 13.8 Hz, 1H), 2.60 (s, 1H), 2.53-2.38 (m, 2H), 1.80-1.66 (m, 2H), 1.59-1.43 (m, 2H), 1.41-1.17 (m, 20H), 1.00-0.79 (m, 6H).

### Example 16: (2R,3S,5R)-5-(6-amino-2-fluoro-9H-purin-9-yl)-2-ethynyl-2-(hydroxymethyl)tetrahydrofuran-3-yl cyclohexanecarboxylate

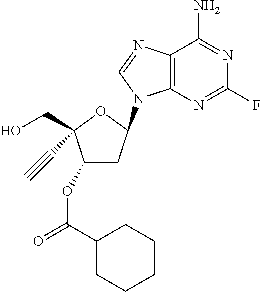 [SMILES: C#C[C@]1(CO)O[C@@H](n2cnc3c(N)nc(F)nc32)C[C@@H]1OC(=O)C1CCCCC1]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting cyclohexanecarboxylic acid for 2-hexyldecanoic acid in step A. LCMS (ESI) m/z calcd for C₂₀H₂₄FN₅O₄: 403.2. Found: 404.3 (M+1)⁺. ¹H NMR (400 MHz, METHANOL-d₄) δ 8.26 (s, 1H), 6.43 (dd, J=6.4, 7.6 Hz, 1H), 5.68 (dd, J=3.6, 6.7 Hz, 1H), 3.94-3.74 (m, 2H), 3.14 (s, 1H), 3.09-2.90 (m, 1H), 2.60 (ddd, J=3.6, 6.3, 13.9 Hz, 1H), 2.43 (tt, J=3.7, 11.1 Hz, 1H), 2.11-1.90 (m, 2H), 1.87-1.74 (m, 2H), 1.72-1.62 (m, 1H), 1.60-1.43 (m, 2H), 1.42-1.20 (m, 3H).

### Example 17: (2R,3S,5R)-5-(6-amino-2-fluoro-9H-purin-9-yl)-2-ethynyl-2-(hydroxymethyl)tetrahydrofuran-3-yl 2-butyloctanoate

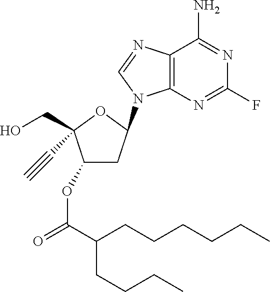 [SMILES: C#C[C@]1(CO)O[C@@H](n2cnc3c(N)nc(F)nc32)C[C@@H]1OC(=O)C(CCCC)CCCCCC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting 2-butyloctanoic acid for 2-hexyldecanoic acid in step A. LCMS (ESI) m/z calcd for C₂₄H₃₄FN₅O₄: 475.3. Found: 476.4 (M+1)⁺. ¹H NMR (400 MHz, CDCl₃) δ 7.87 (s, 1H), 6.33 (dd, J=5.7, 8.6 Hz, 1H), 6.21-5.83 (m, 2H), 5.77 (dd, J=2.5, 6.3 Hz, 1H), 5.45-5.15 (m, 1H), 4.20-3.80 (m, 2H), 3.18 (ddd, J=6.4, 8.6, 13.8 Hz, 1H), 2.60 (d, J=0.7 Hz, 1H), 2.55-2.36 (m, 2H), 1.81-1.67 (m, 2H), 1.60-1.46 (m, 2H), 1.44-1.19 (m, 12H), 0.98-0.83 (m, 6H).

### Example 18: (2R,3S,5R)-5-(6-amino-2-fluoro-9H-purin-9-yl)-2-ethynyl-2-(hydroxymethyl)tetrahydrofuran-3-yl 2,2-dimethylpentanoate

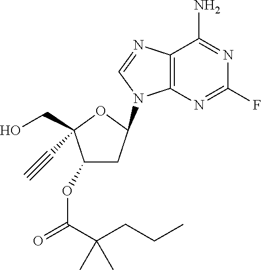 [SMILES: C#C[C@]1(CO)O[C@@H](n2cnc3c(N)nc(F)nc32)C[C@@H]1OC(=O)C(C)(C)CCC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting 2,2-dimethylpentanoic acid for 2-hexyldecanoic acid in step A. LCMS (ESI) m/z calcd for C₁₉H₂₄FN₅O₄: 405.2. Found: 406.3 (M+1)⁺. ¹H NMR (400 MHz, DMSO-d₆) δ 8.34 (s, 1H), 7.87 (br s, 2H), 6.31 (t, J=7.0 Hz, 1H), 5.59-5.45 (m, 2H), 3.77-3.64 (m, 2H), 3.64-3.54 (m, 1H), 3.02 (td, J=6.9, 14.3 Hz, 1H), 2.48-2.41 (m, 1H), 1.59-1.45 (m, 2H), 1.41-1.07 (m, 8H), 0.88 (t, J=7.2 Hz, 3H).

### Example 19: (2R,3S,5R)-5-(6-amino-2-fluoro-9H-purin-9-yl)-2-ethynyl-2-(hydroxymethyl)tetrahydrofuran-3-yl benzoate

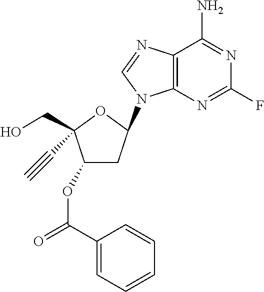 [SMILES: C#C[C@]1(CO)O[C@@H](n2cnc3c(N)nc(F)nc32)C[C@@H]1OC(=O)c1ccccc1]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting benzoic acid for 2-hexyldecanoic acid in step A. LCMS (ESI) m/z calcd for C₁₉H₁₆FN₅O₄: 397.1. Found: 398.2 (M+1)⁺. ¹H NMR (400 MHz, DMSO-d₆) δ 8.37 (s, 1H), 8.13-8.04 (m, 2H), 7.88 (brs, 2H), 7.75-7.66 (m, 1H), 7.62-7.52 (m, 2H), 6.46 (dd, J=6.3, 8.0 Hz, 1H), 5.83 (dd, J=3.1, 6.4 Hz, 1H), 5.66-5.57 (m, 1H), 3.82-3.65 (m, 2H), 3.62 (s, 1H), 3.19-3.07 (m, 1H), 2.72 (ddd, J=3.1, 6.3, 14.0 Hz, 1H).

### Example 20: (2R,3S,5R)-5-(6-amino-2-fluoro-9H-purin-9-yl)-2-ethynyl-2-(hydroxymethyl)tetrahydrofuran-3-yl butyrate

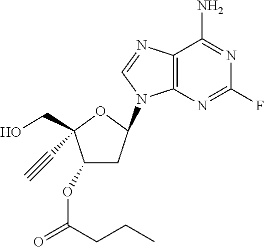 [SMILES: C#C[C@]1(CO)O[C@@H](n2cnc3c(N)nc(F)nc32)C[C@@H]1OC(=O)CCC]

The title compound was isolated as a by-product in the purification of (2R,3S,5R)-5-(6-butyramido-2-fluoro-9H-purin-9-yl)-2-ethynyl-2-(hydroxymethyl)tetrahydrofuran-3-yl butyrate, described herein. LCMS (ESI) m/z calcd for C₁₆H₁₈FN₅O₄: 363.1. Found: 364.1 (M+1)⁺. ¹H NMR (400 MHz, DMSO-d₆) δ 8.33 (s, 1H), 7.87 (brs, 2H), 6.32 (dd, J=6.2, 7.9 Hz, 1H), 5.60 (dd, J=3.2, 6.6 Hz, 1H), 5.54 (dd, J=5.5, 6.9 Hz, 1H), 3.75-3.56 (m, 3H), 3.00 (ddd, J=6.8, 7.7, 14.1 Hz, 1H), 2.57-2.52 (m, 1H), 2.43-2.33 (m, 2H), 1.62 (m, 2H), 0.94 (t, J=7.4 Hz, 3H).

### Example 21: (2R,3S,5R)-5-(6-amino-2-fluoro-9H-purin-9-yl)-2-ethynyl-2-(hydroxymethyl)tetrahydrofuran-3-yl 3-(2-acetoxy-4,6-dimethylphenyl)-3-methylbutanoate

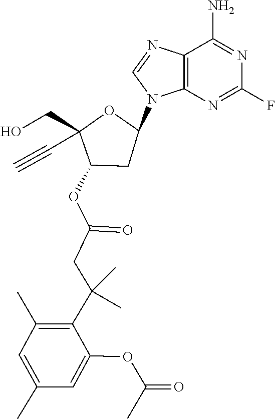 [SMILES: C#C[C@]1(CO)O[C@@H](n2cnc3c(N)nc(F)nc32)C[C@@H]1OC(=O)CC(C)(C)c1c(C)cc(C)cc1OC(C)=O]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting 3-(2-acetoxy-4,6-dimethylphenyl)-3-methylbutanoic acid for 2-hexyldecanoic acid in step A. LCMS (ESI) m/z calcd for C₂₇H₃₀FN₅O₆: 539.2. Found: 540.3 (M+1)⁺. ¹H NMR (400 MHz, DMSO-d₆) δ 8.29 (s, 1H), 7.86 (br s, 2H), 6.86-6.83 (m, 1H), 6.63-6.60 (m, 1H), 6.13 (dd, J=6.3, 7.7 Hz, 1H), 5.48 (dd, J=5.7, 6.7 Hz, 1H), 5.43 (dd, J=3.5, 6.3 Hz, 1H), 3.67-3.59 (m, 2H), 3.58-3.47 (m, 1H), 3.05 (d, J=15.7 Hz, 1H), 2.92-2.73 (m, 2H), 2.54 (s, 3H), 2.29 (s, 3H), 2.17-2.05 (m, 4H), 1.53 (d, J=12.4 Hz, 6H).

### Example 22: (2R,3S,5R)-5-(6-amino-2-fluoro-9H-purin-9-yl)-2-ethynyl-2-(hydroxymethyl)tetrahydrofuran-3-yl (1r,4S)-4-(tert-butyl)cyclohexane-1-carboxylate

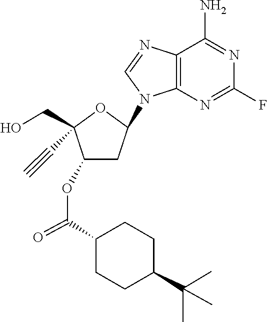 [SMILES: C#C[C@]1(CO)O[C@@H](n2cnc3c(N)nc(F)nc32)C[C@@H]1OC(=O)[C@H]1CC[C@H](C(C)(C)C)CC1]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl 2-hexyldecanoate, substituting trans-4-(tert-butyl)cyclohexane-1-carboxylic acid for 2-hexyldecanoic acid in step A. LCMS (ESI) m/z calcd for C₂₃H₃₀FN₅O₄: 459.2. Found: 460.4 (M+1)⁺. ¹H NMR (400 MHz, CDCl₃) δ 7.85 (s, 1H), 6.33 (dd, J=5.7, 9.1 Hz, 1H), 5.98 (brs, 2H), 5.76 (dd, J=1.9, 6.2 Hz, 1H), 5.40 (br dd, J=2.9, 11.0 Hz, 1H), 4.09-3.88 (m, 2H), 3.19 (ddd, J=6.2, 9.1, 13.8 Hz, 1H), 2.62 (s, 1H), 2.45 (ddd, J=1.9, 5.5, 13.8 Hz, 1H), 2.32 (tt, J=3.6, 12.3 Hz, 1H), 2.19-2.06 (m, 2H), 1.95-1.83 (m, 2H), 1.55-1.41 (m, 2H), 1.12-0.98 (m, 3H), 0.88 (s, 9H).

### Example 23: (2R,3S,5R)-5-(6-amino-2-fluoro-9H-purin-9-yl)-2-ethynyl-2-((((hexyloxy)carbonyl)oxy)methyl)tetrahydrofuran-3-yl tetradecanoate

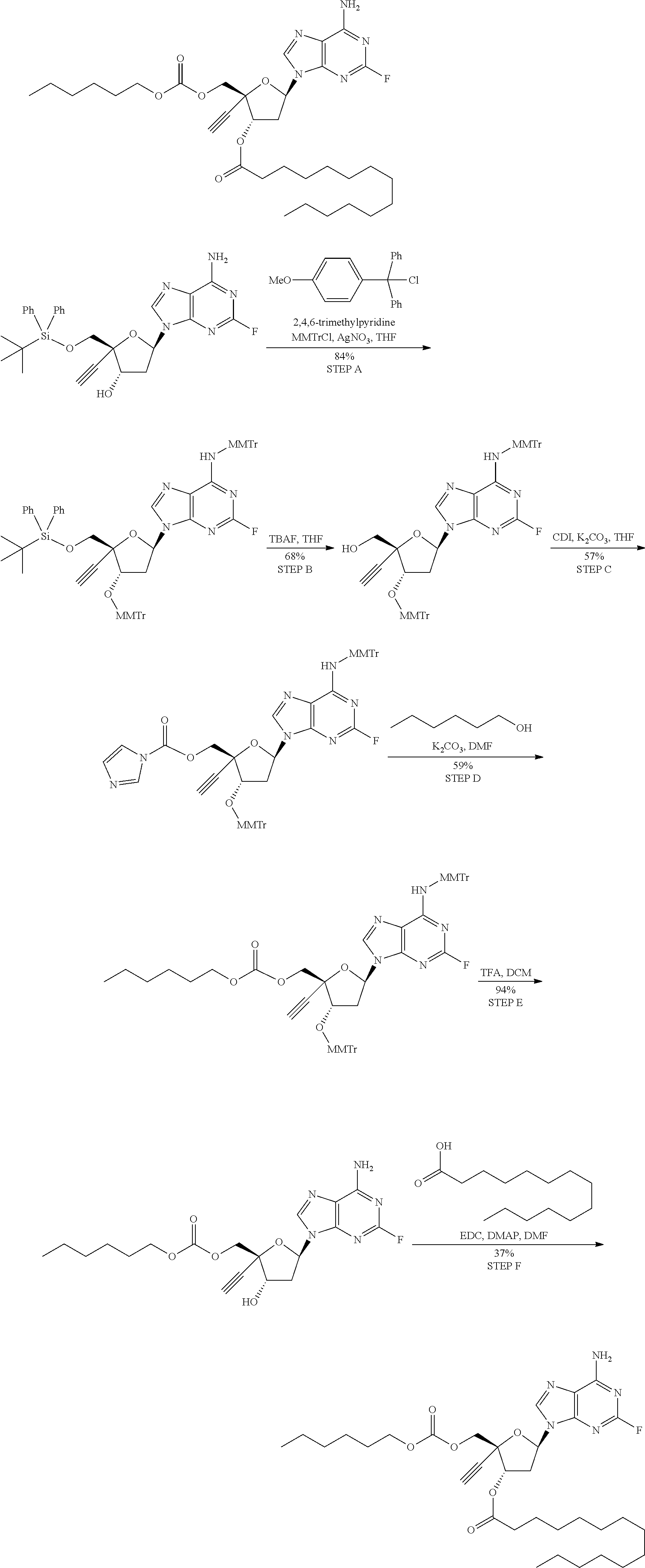

### Step A: 9-((2R,4S,5R)-5-(((tert-butyldiphenylsilyl)oxy)methyl)-5-ethynyl-4-((4-methoxyphenyl)diphenylmethoxy)tetrahydrofuran-2-yl)-2-fluoro-N-((4-methoxyphenyl)diphenylmethyl)-9H-purin-6-amine

To a mixture of (2R,3S,5R)-5-(6-amino-2-fluoro-9H-purin-9-yl)-2-(((tert-butyldiphenylsilyl)oxy)methyl)-2-ethynyltetrahydrofuran-3-ol (10.0 g, 18.8 mmol) in DCM (100 mL) was added 2,4,6-trimethylpyridine (45.6 g, 376 mmol), silver nitrate (32.0 g, 188 mmol) and (chloro(4-methoxyphenyl)methylene)dibenzene (58.1 g, 188 mmol) in portions at 0° C. The reaction mixture was stirred for 2 h at 0° C. LCMS indicated complete reaction. The reaction mixture was filtered, the solid was washed with DCM (200 mL). The filtrate was washed with aqueous NaHCO₃ (2×100 mL), brine (100 mL) and dried over Na₂SO₄. After filtration, the solvent was removed under vacuum. The residue was subjected to flash chromatography (silica gel, 1:1 EtOAc/petroleum ether) to give the desired product (17.0 g, 84%) as a white solid. LCMS (ESI) m/z calcd for C₆₆H₆₂FN₅O₅Si: 1075. Found: 1076 (M+1)⁺. ¹H NMR (300 MHz, CDCl₃) δ 7.59-7.55 (m, 4H), 7.52-7.41 (m, 6H), 7.40-7.33 (m, 2H), 7.30-7.18 (m, 24H), 7.04 (br, 1H), 6.82-6.73 (m, 4H), 6.22 (t, J=6 Hz, 1H), 4.75 (t, J=6 Hz, 1H), 3.96-3.85 (m, 2H), 3.76 (d, J=6 Hz, 6H), 1.76-1.70 (m, 2H), 0.88 (s, 9H).

### Step B: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol

To a mixture of 9-((2R,4S,5R)-5-(((tert-butyldiphenylsilyl)oxy)methyl)-5-ethynyl-4-((4-methoxyphenyl)diphenylmethoxy)tetrahydrofuran-2-yl)-2-fluoro-N-((4-methoxyphenyl)diphenylmethyl)-9H-purin-6-amine (17.0 g, 15.8 mmol) in THF (100 mL) was added TBAF (19 mL, 19 mmol, 1N in THF) at 0° C. The reaction mixture was stirred for 1 hour at 25° C. LCMS indicated complete reaction. The reaction mixture was diluted with water (100 mL), extracted with EtOAc (2×100 mL). The combined organic layer was washed with water (100 mL), aqueous NH₄Cl (100 mL), and dried over Na₂SO₄. After filtration, the filtrate was concentrated to dryness under vacuum. The residue was subjected to flash chromatography (silica gel, 1:1 EtOAc/petroleum ether) to give the title compound (12.0 g, 68%) as a white solid. LCMS (ESI) m/z calcd for C₅₂H₄₄FN₅O₅: 837. Found: 838 (M+1)⁺. ¹H NMR (300 MHz, DMSO-d₆) δ 8.05 (d, J=15 Hz, 2H), 7.59-7.51 (m, 4H), 7.40-7.19 (m, 20H), 6.92-6.83 (m, 4H), 6.08 (t, J=6 Hz, 1H), 5.18 (t, J=6 Hz, 1H), 4.55-4.43 (m, 1H), 3.86 (s, 1H), 3.71 (d, J=3 Hz, 6H), 3.64-3.46 (m, 2H), 1.78-1.53 (m, 2H).

### Step C: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl 1H-imidazole-1-carboxylate

((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (2.50 g, 2.98 mmol) was dissolved in THF (25 mL), and the solution treated with CDI (1.45 g, 8.95 mmol) followed by K₂CO₃ (1.24 g, 8.95 mmol). The resulting mixture was stirred for 30 m at RT. LCMS indicated complete reaction. The mixture was filtered, the solid rinsed with THF, and the filtrate was concentrated. The residue was subjected to flash chromatography (silica gel, 1:1 EtOAc/petroleum ether) to give the desired product (1.6 g, 57%) as a white solid. LCMS (ESI) m/z calcd for C₅₆H₄₆FN₇O₆: 931. Found: 932 (M+1)⁺. ¹H NMR (300 MHz, CDCl₃) δ 7.90 (s, 1H), 7.69 (s, 2H), 7.53 (t, J=15 Hz, 4H), 7.40 (d, J=9 Hz, 2H), 7.29-7.19 (m, 10H), 7.15-7.10 (m, 8H), 7.01 (s, 1H), 6.88 (s, 1H), 6.77 (t, J=15 Hz, 4H), 6.07-6.03 (m, 1H), 4.90 (t, J=18 Hz, 1H), 4.45 (t, J=12 Hz, 1H), 4.24 (t, J=12 Hz, 1H), 3.78 (s, 3H), 3.68 (s, 3H), 2.86 (s, 1H), 2.46-2.36 (m, 1H), 2.11-2.06 (m, 1H).

### Step D: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl 1H-imidazole-1-carboxylate

((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl 1H-imidazole-1-carboxylate (1 g, 1.07 mmol) was dissolved in DMF (10 mL) and the solution treated with hexan-1-ol (0.27 mL, 2.15 mmol) followed by K₂CO₃ (0.300 g, 2.15 mmol) and the resulting mixture was stirred for 2 h at RT. LCMS indicated complete reaction. The reaction mixture was quenched with water (20 mL), and extracted with EtOAc (3×10 mL). The organic phases were combined, washed with brine (20 mL), dried over Na₂SO₄ and concentrated under vacuum. The mixture was subjected to preparative TLC (silica gel, 1:1 EtOAc/petroleum ether) to give the desired product (610 mg, 59%) as a white solid. LCMS (ESI) m/z calcd for C₅₉H₅₆FN₅O₇: 965. Found: 966 (M+1)⁺. ¹H NMR (300 MHz, CDCl₃) δ 7.68 (s, 1H), 7.53 (d, J=6 Hz, 4H), 7.41-7.37 (m, 2H), 7.32-7.28 (m, 10H), 7.23-7.15 (m, 8H), 6.81-6.77 (m, 4H), 6.17-6.13 (m, 1H), 4.55 (t, J=15 Hz, 1H), 4.30 (d, J=12 Hz, 1H), 4.12 (d, J=6 Hz, 1H), 3.99-3.96 (m, 1H), 3.76 (d, J=12 Hz, 6H), 2.83 (s, 1H), 2.23-2.14 (m, 1H), 1.80-1.72 (m, 1H), 1.63-1.56 (m, 2H), 1.33-1.23 (m, 8H), 0.89-0.85 (m, 3H).

### Step E: ((2R,3S,5R)-5-(6-amino-2-fluoro-9H-purin-9-yl)-2-ethynyl-3-hydroxytetrahydrofuran-2-yl)methyl hexyl carbonate

((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl hexyl carbonate (600 mg, 0.620 mmol) was dissolved in DCM (5 mL), and the solution was treated with TFA (0.62 mL). The resulting mixture was stirred for 1 h at RT. LCMS indicated complete reaction. The reaction mixture was diluted with MeOH (5 mL), and concentrated to dryness at reduced pressure. The mixture was subjected to preparative TLC (silica gel, 20:1 DCM/MeOH) to give the desired product (250 mg, 94%) as a white solid. LCMS (ESI) m/z calcd for C₁₉H₂₄FN₅O₅: 421. Found: 422 (M+1)⁺. ¹H NMR (300 MHz, METHANOL-d₄) δ 8.14 (s, 1H), 6.34-6.30 (m, 1H), 4.96-4.87 (m, 1H), 4.51 (d, J=12 Hz, 1H), 4.31 (d, J=12 Hz, 1H), 4.07-3.97 (m, 2H), 3.17 (s, 1H), 2.89-2.81 (m, 1H), 2.70-2.61 (m, 1H), 1.61-1.53 (m, 2H), 1.35-1.23 (m, 6H), 0.91-0.86 (m, 3H).

### Step F: (2R,3S,5R)-5-(6-amino-2-fluoro-9H-purin-9-yl)-2-ethynyl-2-((((hexyloxy)carbonyl)oxy)methyl)tetrahydrofuran-3-yl tetradecanoate

Tetradecanoic acid (249 mg, 1.09 mmol) was dissolved in DMF (4 mL) and the solution was treated with DMAP (400 mg, 3.27 mmol) followed by EDC (628 mg, 3.27 mmol). After stirring the solution at RT for 2 h, ((2R,3S,5R)-5-(6-amino-2-fluoro-9H-purin-9-yl)-2-ethynyl-3-hydroxytetrahydrofuran-2-yl)methyl hexyl carbonate (230 mg, 0.55 mmol) was added, and the resulting mixture was stirred overnight at RT. LCMS indicated complete reaction. The reaction mixture was quenched with water (10 mL), extracted with EtOAc (3×5 mL). The organic phases were combined, washed with brine (10 mL), dried over Na₂SO₄ and concentrated under vacuum. The mixture was subjected to preparative RP-HPLC (C18, MeCN/water, 0.05% TFA) to give the desired product (129 mg, 37%) as a white solid. LCMS (ESI) m/z calcd for C₃₃H₅₀FN₅O₆: 631. Found: 632 (M+1)⁺. ¹H NMR (400 MHz, METHANOL-d₄) δ 8.17 (s, 1H), 6.40 (t, J=12 Hz, 1H), 5.82-5.78 (m, 1H), 4.50 (d, J=12 Hz, 1H), 4.39 (d, J=12 Hz, 1H), 4.12-4.01 (m, 2H), 3.27 (s, 1H), 3.11-3.04 (m, 1H), 2.76-2.70 (m, 1H), 2.43 (t, J=16 Hz, 2H), 1.71-1.57 (m, 4H), 1.38-1.23 (m, 26H), 0.90-0.87 (m, 6H).

### Example 24: (2R,3S,5R)-5-(6-amino-2-fluoro-9H-purin-9-yl)-2-(((ethoxycarbonyl)oxy)methyl)-2-ethynyltetrahydrofuran-3-yl stearate

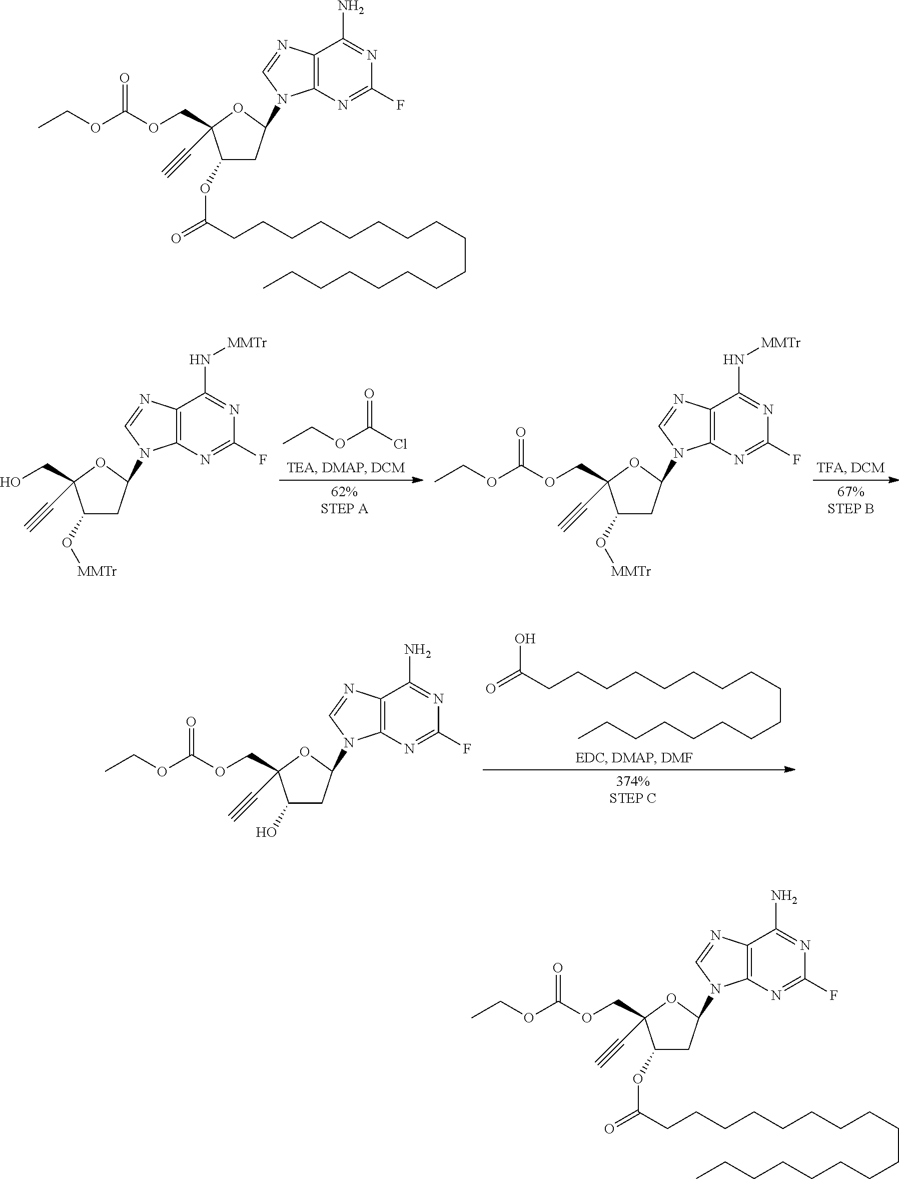

### Step A: ethyl (((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl) carbonate

To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (1.00 g, 1.19 mmol), TEA (1.00 mL, 7.16 mmol) and DMAP (72.9 mg, 0.60 mmol) in DCM (10 mL) was added ethyl chloroformate (1.17 g, 10.7 mmol) dropwise at 25° C. The reaction mixture was stirred at 25° C. for 3 h. LCMS indicated complete reaction. The reaction was quenched by the addition of water (30 mL), and extracted with DCM (3×20 ml). The combined organic layer was dried over Na₂SO₄ and evaporated to dryness in vacuum. The residue was subjected to flash chromatography (silica gel, 2:5 EtOAc/petroleum ether) to give the desired product (730 mg, 62%) as a white solid. LCMS (ESI) m/z calcd for: C₅₅H₄₈FN₅O₇: 909. Found: 910 (M+1)⁺. ¹H NMR (400 MHz, DMSO-d₆) δ 8.08 (s, 1H), 7.99 (s, 1H), 7.60-7.45 (m, 4H), 7.38-7.18 (m, 20H), 6.91-6.77 (m, 4H), 6.14 (dd, J=8.0, 3.6 Hz, 1H), 4.65 (t, J=7.2 Hz, 1H), 4.15 (d, J=11.6 Hz, 1H), 3.96 (s, 1H), 3.71 (d, J=11.6 Hz, 1H), 3.69 (s, 3H), 3.67 (s, 3H), 2.07-2.00 (m, 1H), 1.91-1.87 (m, 3H), 0.75 (t, J=7.2 Hz, 3H).

### Step B: ((2R,3S,5R)-5-(6-amino-2-fluoro-9H-purin-9-yl)-2-ethynyl-3-hydroxytetrahydrofuran-2-yl)methyl ethyl carbonate

To a stirred solution of ethyl (((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl) carbonate (730 mg, 0.80 mmol) in DCM (8 mL) was added TFA (0.80 mL). The reaction mixture was stirred at 25° C. for 30 m. LCMS indicated complete reaction. The reaction was quenched by the addition of methanol until the yellow solution turned to colorless. The resulting mixture was evaporated to dryness in vacuum to give the crude product. The residue was subjected to RP-HPLC purification (C18, MeCN/water, 0.1% formic acid) to give the desired product (197 mg, 67%) as a white solid. LCMS (ESI) m/z calcd for C₁₅H₁₆FN₅O₅: 365. Found: 366 (M+1)⁺. ¹H NMR (400 MHz, DMSO-d₆) δ 8.26 (s, 1H), 7.86 (br s, 2H), 6.26-6.23 (m, 1H), 5.80 (d, J=5.6 Hz, 1H), 4.74-4.68 (m, 1H), 4.42 (d, J=12.0 Hz, 1H), 4.11 (d, J=12.0 Hz, 1H), 3.64 (s, 1H), 2.82-2.76 (m, 1H), 2.49-2.44 (m, 1H), 2.35-2.15 (m, 2H), 1.15 (t, J=7.2 Hz, 3H).

### Step C: (2R,3S,5R)-5-(6-amino-2-fluoro-9H-Dunn-9-yl)-2-(((ethoxycarbonyl)oxy)methyl)-2-ethynyltetrahydrofuran-3-yl stearate

A solution of stearic acid (413 mg, 1.45 mmol), EDC (556 mg, 2.90 mmol) and DMAP (354 mg, 2.90 mmol) in DMF (9 mL) was stirred at 25° C. for 30 m. Then ((2R,3S,5R)-5-(6-amino-2-fluoro-9H-purin-9-yl)-2-ethynyl-3-hydroxytetrahydrofuran-2-yl)methyl ethyl carbonate (265 mg, 0.730 mmol) was added. The reaction mixture was stirred at 25° C. for 20 h. LCMS indicated complete reaction. The reaction was diluted with water (30 ml), and the resulting mixture was extracted with EtOAc (3×20 ml). The combined organic layer was washed with brine (20 mL), dried over Na₂SO₄ and evaporated to dryness under vacuum. The residue was purified by RP-HPLC (C18, MeCN/water, 0.1% formic acid) to give the desired product (159 mg, 34%) as a white solid. LCMS (ESI) m/z calcd for C₃₃H₅₀FN₅O₆: 631. Found: 632 (M+1)⁺. ¹H NMR (400 MHz, DMSO-d₆) δ 8.34 (s, 1H), 7.91 (br s, 2H), 6.35 (t, J=6.8 Hz, 1H), 5.69 (t, J=5.6 Hz, 1H), 4.42 (d, J=11.6 Hz, 1H), 4.20 (d, J=11.6 Hz, 1H), 3.79 (s, 1H), 3.15-3.12 (m, 1H), 2.62-2.60 (m, 1H), 2.50-2.31 (m, 4H), 1.60-1.56 (m, 2H), 1.31-1.23 (m, 28H), 1.17 (t, J=7.2 Hz, 3H), 0.85 (t, J=6.4 Hz, 3H).

### Example 25: (2R,3S,5R)-2-ethynyl-5-(2-fluoro-6-heptanamido-9H-purin-9-yl)-2-(hydroxymethyl)tetrahydrofuran-3-yl heptanoate

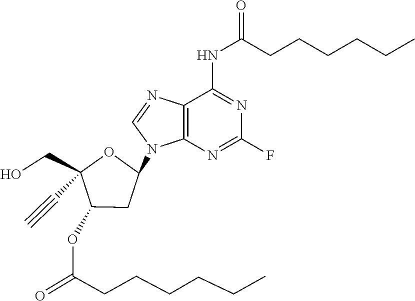 [SMILES: C#C[C@]1(CO)O[C@@H](n2cnc3c(NC(=O)CCCCCC)nc(F)nc32)C[C@@H]1OC(=O)CCCCCC]

The title compound was prepared as described herein for the synthesis of (2R,3S,5R)-5-(6-butyramido-2-fluoro-9H-purin-9-yl)-2-ethynyl-2-(hydroxymethyl)tetrahydrofuran-3-yl butyrate, substituting heptanoic acid for butyric acid. LCMS (ESI) m/z calcd for C₂₆H₃₆FN₅O₅: 517.3. Found: 516.7 (M−1). ¹H NMR (400 MHz, CDCl₃) δ 8.62 (s, 1H), 8.10 (s, 1H), 6.40 (dd, J=5.7, 8.6 Hz, 1H), 5.79 (dd, J=2.6, 6.4 Hz, 1H), 4.66 (dd, J=3.8, 10.5 Hz, 1H), 4.11-3.87 (m, 2H), 3.15 (ddd, J=6.6, 8.5, 13.8 Hz, 1H), 3.03-2.93 (m, 2H), 2.64 (s, 1H), 2.55 (ddd, J=2.6, 5.7, 13.8 Hz, 1H), 2.43 (t, J=7.6 Hz, 2H), 1.82-1.64 (m, 4H), 1.49-1.26 (m, 12H), 0.98-0.85 (m, 6H).

### Example 26: (2R,3S,5R)-5-(6-butyramido-2-fluoro-9H-purin-9-yl)-2-ethynyl-2-(hydroxymethyl)tetrahydrofuran-3-yl butyrate

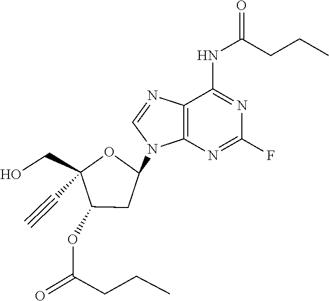 [SMILES: C#C[C@]1(CO)O[C@@H](n2cnc3c(NC(=O)CCC)nc(F)nc32)C[C@@H]1OC(=O)CCC]

A suspension of (2R,3S,5R)-5-(6-amino-2-fluoro-9H-purin-9-yl)-2-(((tert-butyldiphenylsilyl)oxy)methyl)-2-ethynyltetrahydrofuran-3-ol (800 mg, 1.51 mmol) in DCM (15 mL) was treated with butyric acid (0.275 mL, 3.01 mmol), DMAP (184 mg, 1.51 mmol), EDC (865 mg, 4.51 mmol), DIEA (1.31 mL, 7.52 mmol), and stirred at RT for 1.5 h. The reaction was concentrated and the residue subjected to flash chromatography (silica gel, 0-100% EtOAc/DCM) to give a mixture of (2R,3S,5R)-2-(((tert-butyldiphenylsilyl)oxy)methyl)-5-(6-butyramido-2-fluoro-9H-purin-9-yl)-2-ethynyltetrahydrofuran-3-yl butyrate and (2R,3S,5R)-5-(6-amino-2-fluoro-9H-purin-9-yl)-2-(((tert-butyldiphenylsilyl)oxy)methyl)-2-ethynyltetrahydrofuran-3-yl butyrate. The mixture was subjected to TBAF/THF deprotection as described herein followed by flash chromatography [0-100% (3:1 EtOAc:EtOH)/hexanes] to afford the title compound and (2R,3S,5R)-5-(6-amino-2-fluoro-9H-purin-9-yl)-2-ethynyl-2-(hydroxymethyl)tetrahydrofuran-3-yl butyrate, both as white solids. Data for (2R,3S,5R)-5-(6-butyramido-2-fluoro-9H-purin-9-yl)-2-ethynyl-2-(hydroxymethyl)tetrahydrofuran-3-yl butyrate: LCMS (ESI) m/z calcd for C₂₀H₂₄FN₅O₅: 433.2. Found: 434.2 (M+1)⁺. ¹H NMR (400 MHz, CDCl₃) δ 8.82 (s, 1H), 8.14 (s, 1H), 6.41 (dd, J=5.7, 8.6 Hz, 1H), 5.80 (dd, J=2.7, 6.3 Hz, 1H), 4.68 (brs, 1H), 4.08-4.00 (m, 1H), 4.00-3.89 (m, 1H), 3.14 (ddd, J=6.6, 8.5, 13.8 Hz, 1H), 2.97 (t, J=7.3 Hz, 2H), 2.64 (s, 1H), 2.56 (ddd, J=2.6, 5.8, 13.8 Hz, 1H), 2.42 (t, J=7.4 Hz, 2H), 1.88-1.69 (m, 4H), 1.12-0.96 (m, 6H).

### Example 27: (2R,3S,5R)-5-(6-decanamido-2-fluoro-9H-purin-9-yl)-2-ethynyl-2-(hydroxymethyl)tetrahydrofuran-3-yl decanoate

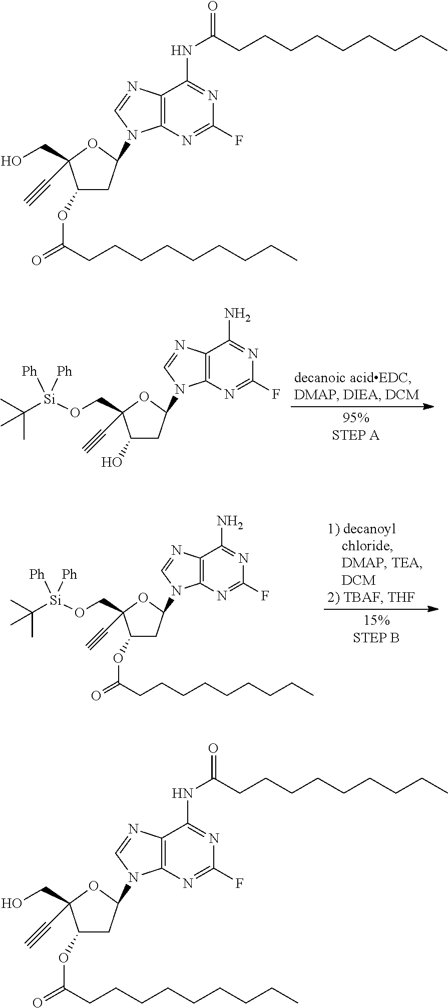

### Step A: (2R,3S,5R)-5-(6-amino-2-fluoro-9H-purin-9-yl)-2-(((tert-butyldiphenylsilyl)oxy)methyl)-2-ethynyltetrahydrofuran-3-yl decanoate

To a suspension of (2R,3S,5R)-5-(6-amino-2-fluoro-9H-purin-9-yl)-2-(((tert-butyldiphenylsilyl)oxy) methyl)-2-ethynyltetrahydrofuran-3-ol (550 mg, 1.024 mmol) in dichloromethane (16 mL) was added decanoic acid (353 mg, 2.05 mmol) followed by DMAP (138 mg, 1.13 mmol), EDC (589 mg, 3.07 mmol) and DIEA (0.894 mL, 5.12 mmol) and the mixture was stirred at ambient temperature for 18 h. The mixture was diluted with DCM and washed with water. The organic phase was dried (Na₂SO₄), concentrated and purified on silica gel (EtOAc/hexanes 0-100%) to provide (2R,3S,5R)-5-(6-amino-2-fluoro-9H-purin-9-yl)-2-(((tert-butyldiphenyl silyl)oxy) methyl)-2-ethynyltetrahydrofuran-3-yl decanoate (670 mg, 95%) as an off-white foam. LCMS (ESI) m/z calcd for C₃₈H₄₈FN₅O₄Si: 685.4. Found: 686.9 (M+1)⁺. ¹H NMR (400 MHz, CDCl₃) δ 8.10 (s, 1H), 7.66 (td, J=8.1, 1.4 Hz, 4H), 7.38-7.58 (m, 6H), 6.48 (t, J=6.4 Hz, 1H), 5.76 (dd, J=6.3, 5.1 Hz, 1H), 3.98-4.23 (m, 2H), 2.73-2.94 (m, 2H), 2.63 (s, 1H), 2.35-2.48 (m, 2H), 1.61-1.78 (m, 2H), 1.21-1.48 (m, 12H), 1.12 (s, 9H), 0.91 (s, 3H).

### Step B: (2R,3S,5R)-5-(6-decanamido-2-fluoro-9H-purin-9-yl)-2-ethynyl-2-(hydroxymethyl)tetrahydrofuran-3-yl decanoate

To a solution of (2R,3S,5R)-5-(6-amino-2-fluoro-9H-purin-9-yl)-2-(((tert-butyldiphenylsilyl)oxy) methyl)-2-ethynyltetrahydrofuran-3-yl decanoate (665 mg, 0.970 mmol) in DCM (13 mL) was added TEA (0.270 mL, 1.939 mmol) and DMAP (118 mg, 0.970 mmol). The solution was cooled to 0° C. and decanoyl chloride (0.302 mL, 1.45 mmol) was added and after 5 min the solution was warmed to RT and stirred for 4.5 h. The mixture was cooled to 0° C. and TEA (0.270 mL, 1.94 mmol) and DMAP (118 mg, 0.970 mmol) was added followed by dropwise addition of decanoyl chloride (0.302 mL, 1.45 mmol). The mixture was diluted with DCM and washed with saturated NaHCO₃/water. The organic phase was dried (Na₂SO₄), concentrated and the residue purified on silica gel (EtOAc/hexanes, 0-100%) to provide (2R,3S,5R)-2-(((tert-butyldiphenylsilyl)oxy)methyl)-5-(6-decanamido-2-fluoro-9H-purin-9-yl)-2-ethynyltetrahydrofuran-3-yl decanoate. This material (220 mg, 0.262 mmol) was dissolved in THF (4 mL). The solution was cooled to 0° C. and was treated with 1M TBAF/THF (0.524 mL, 0.524 mmol). After stirring at RT for 4 h, the solution was treated with acetic acid (0.031 mL, 0.550 mmol), diluted with DCM, and washed with water. The organic phase was dried (Na₂SO₄), concentrated and purified on silica gel (EtOAc/hexanes 0-100%) to provide a clear glass. This residue was dissolved in MeOH and few drops of water. Slow evaporation provided the title compound as an off-white solid in 42% yield (2 steps). LCMS (ESI) m/z calcd for C₃₂H₄₈FN₅O₅: 601.4. Found: 602.4 (M+1)⁺. ¹H NMR (400 MHz, CDCl₃) δ 8.55 (s, 1H), 8.07 (s, 1H), 6.41 (dd, J=8.6, 5.7 Hz, 1H), 5.80 (dd, J=6.4, 2.6 Hz, 1H), 4.66 (dd, J=10.6, 3.9 Hz, 1H), 4.02-4.11 (m, 1H), 3.90-4.00 (m, 1H), 3.16 (ddd, J=13.8, 8.6, 6.7 Hz, 1H), 2.98 (t, J=7.5 Hz, 2H), 2.65 (s, 1H), 2.56 (ddd, J=13.8, 5.7, 2.6 Hz, 1H), 2.44 (t, J=7.5 Hz, 2H), 1.65-1.88 (m, 4H), 1.22-1.51 (m, 24H), 0.85-0.97 (m, 6H).

### Example 28: (2R,3S,5R)-2-ethynyl-5-(2-fluoro-6-tetradecanamido-9H-purin-9-yl)-2-(hydroxymethyl)tetrahydrofuran-3-yl 3-(2-acetoxy-4,6-dimethylphenyl)-3-methylbutanoate

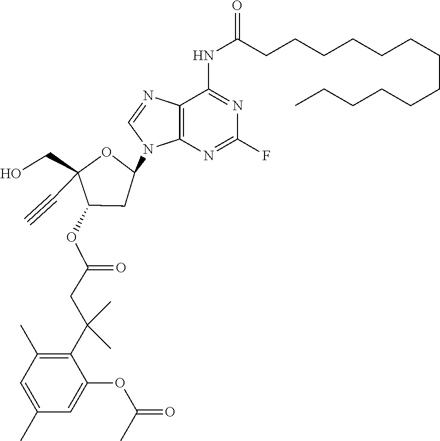 [SMILES: C#C[C@]1(CO)O[C@@H](n2cnc3c(NC(=O)CCCCCCCCCCCCC)nc(F)nc32)C[C@@H]1OC(=O)CC(C)(C)c1c(C)cc(C)cc1OC(C)=O]

The title compound was prepared as described herein for the synthesis of (2R,3S,5R)-5-(6-decanamido-2-fluoro-9H-purin-9-yl)-2-ethynyl-2-(hydroxymethyl)tetrahydrofuran-3-yl decanoate, using 3-(2-acetoxy-4,6-dimethylphenyl)-3-methylbutanoic acid and tetradecanoyl chloride for steps A and B respectively. LCMS (ESI) m/z calcd for C₄₁H₅₆FN₅O₇: 749.4. Found: 750.5 (M+1)⁺. ¹H NMR (400 MHz, DMSO-d₆) δ 10.98 (s, 1H), 8.60 (s, 1H), 6.84 (d, J=2.1 Hz, 1H), 6.62 (d, J=1.9 Hz, 1H), 6.27-6.22 (m, 1H), 5.47 (dd, J=3.9, 6.6 Hz, 2H), 3.67-3.59 (m, 2H), 3.58-3.52 (m, 1H), 3.05 (d, J=15.5 Hz, 1H), 2.96-2.87 (m, 1H), 2.81 (d, J=15.7 Hz, 1H), 2.61-2.53 (m, 4H), 2.29 (s, 3H), 2.25-2.15 (m, 1H), 2.12 (s, 3H), 1.65-1.49 (m, 7H), 1.37-1.13 (m, 22H), 0.91-0.79 (m, 3H).

### Example 29: (2R,3S,5R)-5-(6-butyramido-2-fluoro-9H-purin-9-yl)-2-ethynyl-2-(hydroxymethyl)tetrahydrofuran-3-yl heptadecanoate

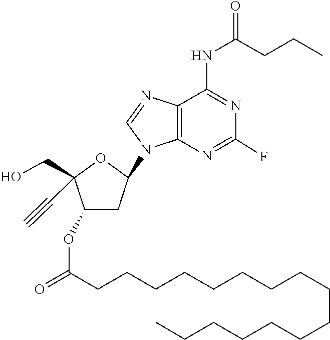 [SMILES: C#C[C@]1(CO)O[C@@H](n2cnc3c(NC(=O)CCC)nc(F)nc32)C[C@@H]1OC(=O)CCCCCCCCCCCCCCCC]

The title compound was prepared as described herein for the synthesis of (2R,3S,5R)-5-(6-decanamido-2-fluoro-9H-purin-9-yl)-2-ethynyl-2-(hydroxymethyl)tetrahydrofuran-3-yl decanoate, using heptadecanoic acid and butyryl chloride in steps A and B respectively. LCMS (ESI) m/z calcd for C₃₃H₅₀FN₅O₅: 615. Found: 616 (M+1)⁺. ¹H NMR (400 MHz, DMSO-d₆) δ 8.85 (s, 1H), 8.14 (s, 1H), 6.43-6.40 (m, 1H), 5.80-5.77 (m, 1H), 4.83 (s, 1H), 4.03 (d, J=12.8 Hz, 1H), 3.94 (d, J=12.4 Hz, 1H), 3.10-3.15 (m, 1H), 2.96 (t, J=7.6 Hz, 2H), 2.63 (s, 1H), 2.53-2.57 (m, 1H), 2.42 (t, J=7.6 Hz, 2H), 1.77-1.82 (m, 2H), 1.65-1.70 (m, 2H), 1.25-1.32 (m, 26H), 1.05 (t, J=7.2 Hz, 3H), 0.89-0.86 (m, 3H).

### Example 30: (2R,3S,5R)-2-ethynyl-5-(2-fluoro-6-octanamido-9H-purin-9-yl)-2-(hydroxymethyl)tetrahydrofuran-3-yl tridecanoate

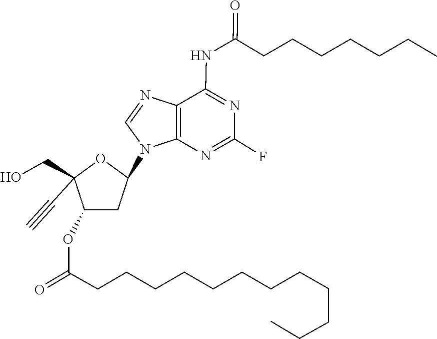 [SMILES: C#C[C@]1(CO)O[C@@H](n2cnc3c(NC(=O)CCCCCCC)nc(F)nc32)C[C@@H]1OC(=O)CCCCCCCCCCCC]

The title compound was prepared as described herein for the synthesis of (2R,3S,5R)-5-(6-decanamido-2-fluoro-9H-purin-9-yl)-2-ethynyl-2-(hydroxymethyl)tetrahydrofuran-3-yl decanoate, using tridecanoic acid and octanoyl chloride in steps A and B respectively. LCMS (ESI) m/z calcd for C₃₃H₅₀FN₅O₅: 615. Found: 616 (M+1)⁺. ¹H NMR (400 MHz, CDCl₃) δ 8.64 (s, 1H), 8.08 (s, 1H), 6.41 (dd, J=8.4, 5.6 Hz, 1H), 5.78 (dd, J=6.0, 2.0 Hz, 1H), 4.05-3.92 (m, 2H), 3.17-3.11 (m, 1H), 2.97 (t, J=7.2 Hz, 2H), 2.63 (s, 1H), 2.56-2.51 (m, 1H), 2.44 (t, J=7.6 Hz, 2H), 1.79-1.72 (m, 2H), 1.70-1.65 (m, 2H), 1.45-1.26 (m, 26H), 0.90-0.86 (m, 6H).

### Example 31: (2R,3S,5R)-2-ethynyl-5-(2-fluoro-6-pentanamido-9H-purin-9-yl)-2-(hydroxymethyl)tetrahydrofuran-3-yl palmitate

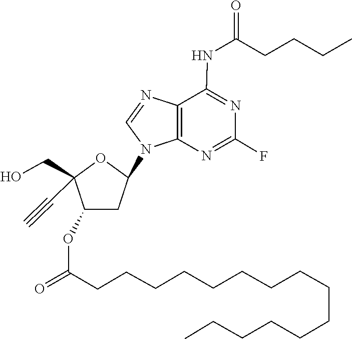 [SMILES: C#C[C@]1(CO)O[C@@H](n2cnc3c(NC(=O)CCCC)nc(F)nc32)C[C@@H]1OC(=O)CCCCCCCCCCCCCCC]

The title compound was prepared as described herein for the synthesis of (2R,3S,5R)-5-(6-decanamido-2-fluoro-9H-purin-9-yl)-2-ethynyl-2-(hydroxymethyl)tetrahydrofuran-3-yl decanoate, using palmitic acid and pentanoyl chloride in steps A and B respectively. LCMS (ESI) m/z calcd for C₃₃H₅₀FN₅O₅: 615. Found: 616 (M+1)⁺. ¹H NMR (400 MHz, CDCl₃) δ 8.94 (s, 1H), 8.16 (s, 1H), 6.41 (dd, J=8.4, 5.6 Hz, 1H), 5.78 (dd, J=6.4, 2.4 Hz, 1H), 4.05-3.92 (m, 2H), 3.16-3.09 (m, 1H), 2.97 (t, J=7.2 Hz, 2H), 2.63 (s, 1H), 2.57-2.52 (m, 1H), 2.42 (t, J=7.6 Hz, 2H), 1.78-1.64 (m, 4H), 1.48-1.25 (m, 26H), 0.96 (t, J=7.2 Hz, 3H), 0.87 (t, J=6.8 Hz, 3H).

### Example 32: (2R,3S,5R)-2-ethynyl-5-(2-fluoro-6-hexanamido-9H-purin-9-yl)-2-(hydroxymethyl)tetrahydrofuran-3-yl pentadecanoate

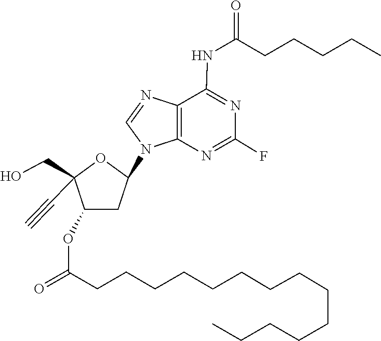 [SMILES: C#C[C@]1(CO)O[C@@H](n2cnc3c(NC(=O)CCCCC)nc(F)nc32)C[C@@H]1OC(=O)CCCCCCCCCCCCCC]

The title compound was prepared as described herein for the synthesis of (2R,3S,5R)-5-(6-decanamido-2-fluoro-9H-purin-9-yl)-2-ethynyl-2-(hydroxymethyl)tetrahydrofuran-3-yl decanoate, using pentadecanoic acid and hexanoyl chloride in steps A and B respectively. LCMS (ESI) m/z calcd for C₃₃H₅₀FN₅O₅: 615. Found: 616 (M+1). ¹H NMR (400 MHz, CDCl₃) δ 8.80 (s, 1H), 8.13 (s, 1H), 6.40 (dd, J=8.4, 5.6 Hz, 1H), 5.78 (dd, J=6.4, 2.4 Hz, 1H), 4.05-3.92 (m, 2H), 3.17-3.10 (m, 1H), 2.96 (t, J=7.6 Hz, 2H), 2.63 (s, 1H), 2.57-2.51 (m, 1H), 2.42 (t, J=7.6 Hz, 2H), 1.80-1.64 (m, 4H), 1.43-1.25 (m, 26H), 0.93-0.86 (m, 6H).

### Example 33: (2R,3S,5R)-2-ethynyl-5-(2-fluoro-6-heptanamido-9H-purin-9-yl)-2-(hydroxymethyl)tetrahydrofuran-3-yl tetradecanoate

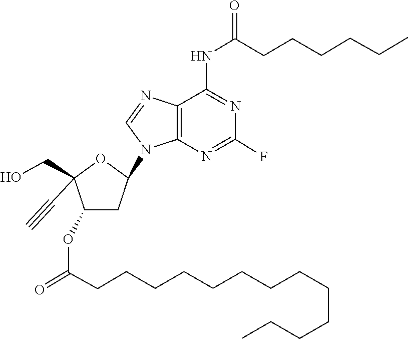 [SMILES: C#C[C@]1(CO)O[C@@H](n2cnc3c(NC(=O)CCCCCC)nc(F)nc32)C[C@@H]1OC(=O)CCCCCCCCCCCCC]

The title compound was prepared as described herein for the synthesis of (2R,3S,5R)-5-(6-decanamido-2-fluoro-9H-purin-9-yl)-2-ethynyl-2-(hydroxymethyl)tetrahydrofuran-3-yl decanoate, using tetradecanoic acid and heptanoyl chloride in steps A and B respectively. LCMS (ESI) m/z calcd for C₃₃H₅₀FN₅O₅: 615, Found: 616 (M+1)⁺. ¹H NMR (400 MHz, CDCl₃) δ 8.68 (s, 1H), 8.12 (s, 1H), 6.40 (dd, J=8.8, 5.6 Hz, 1H), 5.78 (dd, J=6.4, 2.0 Hz, 1H), 4.75 (brs, 1H), 4.05-3.92 (m, 2H), 3.17-3.10 (m, 1H), 2.95 (t, J=7.2 Hz, 2H), 2.63 (s, 1H), 2.57-2.51 (m, 1H), 2.42 (t, J=7.6 Hz, 2H), 1.79-1.71 (m, 2H), 1.70-1.64 (m, 2H), 1.44-1.26 (m, 26H), 0.91-0.86 (m, 6H).

### Evaluating Anti-HIV Activity

PSV Assay

A pseudotyped virus assay was used to assess the potency of each HIV inhibitor. The replication defective pseudovirus (PSV) was produced by co-transfection of a plasmid containing an NL4-3 provirus [containing a mutation in the envelope open reading frame (ORF) and a luciferase reporter gene replacing the nef ORF], and a CMV-promoter expression plasmid containing an ORF for various HIV gp160 envelope clones. The harvested virus was stored at −80 C in small aliquots and the titer of the virus measured to produce a robust signal for antiviral assays. Two replication defective PSVs were used: one containing the wild-type NL4-3 and a second that has a methionine 184 to valine (M184V) substitution in the reverse transcriptase gene. Resistance to reverse transcriptase inhibitors such as lamivudine(3TC) is associated with the M184V mutation (Wainberg et. al. Science 1996: Enhanced fidelity of 3TC-selected mutant HIV-1 reverse transcriptase).

The PSV assay was performed using U373 cells stably transformed to express human CD4, the primary receptor for HIV entry, and either human CXCR4 or human CCR5 which are co-receptors for HIV entry. The below-referenced molecules of interest are serially diluted into tissue culture media to create a dose range of concentrations. This dose-range was applied to U373 cells and the pre-made pseudotyped virus added. The amount of luciferase signal produced after 3 days of culture was used to reflect the level of pseudotyped virus infection. An IC50, or the concentration of inhibitor required to reduce PSV infection by 50% from the infection containing no inhibitor was calculated. Assays to measure cytotoxity were performed in parallel to ensure the antiviral activity observed for an inhibitor was distinguishable from reduced target cell viability. IC₅₀ values were determined from a 10-point dose response curve using 4-fold serial dilution for each compound, which spans a concentration range >1000 fold.

These values are plotted against the molar compound concentrations using the logistic equation: y=((Vmax*x)/(K+x))+Y2 where: Y2=minimum y; Vmax=maximum y; x=compound concentration[M] and K=IC₅₀. The mean IC₅₀ values for each compound, the number of independent assay runs, along with the fold change of the IC₅₀ of the M184V mutant vs WT NL4-3 are presented in Tables 3 and 4.

TABLE 3

| Example | WT IC₅₀ (μM) | M184V IC50 (μM) |
|---|---|---|
| EFdA | 0.0065 | 0.0351 |
| 1 | 0.1055 | 0.5378 |
| 2 | 0.0015 | 0.0078 |
| 3 | 0.0013 | 0.0070 |
| 4 | 0.0041 | 0.0240 |
| 5 | 0.0355 | 0.1682 |
| 6 | 0.6353 | 3.2781 |
| 7 | 0.0071 | 0.0364 |
| 8 | 0.1651 | 0.4601 |
| 9 | 0.0114 | 0.0530 |

TABLE 4

| Example | WT IC₅₀ (μM) | M184V IC50 (μM) |
|---|---|---|
| 10 | 0.0072 | 0.0262 |
| 11 | 0.0029 | 0.0170 |
| 12 | 0.0018 | 0.0107 |
| 13 | 0.2625 | 1.2770 |
| 14 | 0.0114 | 0.0680 |
| 15 | 0.5546 | 3.1160 |
| 16 | 0.0039 | 0.0190 |
| 17 | 0.1454 | 0.5982 |
| 18 | 0.0464 | 0.1679 |
| 19 | 0.0408 | 0.1973 |
| 20 | 0.0277 | 0.1235 |
| 22 | 0.0026 | 0.0093 |
| 23 | 1.0930 | 1.1310 |
| 24 | 1.2660 | 2.1240 |
| 25 | 0.0094 | 0.0439 |
| 26 | 0.1020 | 0.4652 |
| 27 | 0.0068 | 0.0286 |
| 28 | 0.0415 | 0.1532 |

### Antiviral Persistence Assay

The PSV assay was adapted to determine the antiviral persistence of each compound. This assay evaluates the ability of each compound to remain active in cells for two days i.e prevent PSV infection of cells in a dose dependent manner, 48 h after the removal of compound. Duplicate plates of U373 cells were treated with a serial dilution of small molecule inhibitors for 6 h at 37° C. Compounds were removed from cells by washing twice cells with 1×PBS. For baseline group (i.e immediately after washing or 0 h), cells were infected with prepared PSVs and cultured for three days. For experimental group (48 h), the culture medium is added to the washed cells and the plate incubated at 37° C. for 48 h. After two days of culture, the prepared PSVs were added to the cells and the mixture cultured for three days. The amount of luciferase signal produced after culture was used to reflect the level of pseudotyped virus infection in the baseline group (0 h) and experimental group (48 h) for each compound. An IC₅₀, or the concentration of inhibitor required to reduce PSV infection by 50% from the infection containing no inhibitor was calculated. The persistence index, which is the ratio of the IC₅₀ determined at 48 and 0 h is presented in Table 5 as well as the fold change of the persistence index relative to EFdA [(2R,3S,5R)-5-(6-amino-2-fluoro-9H-purin-9-yl)-2-ethynyl-2-(hydroxymethyl)tetrahydrofuran-3-ol].

Statistical analysis and graphing of the data were performed in JMP 13.2.1 (SAS Institute, Cary, N.C.). A four-parameter logistic Hill Model was fit to % Inhibition and log₁₀ concentration values, separately for each compound, time point and run. A pilot experiment included 2 independent experimental runs and a later follow-up experiment included 4 runs. Quality control criteria based on R² and 95% confidence interval ranges of all four parameter estimates were used to exclude curves with poor fits. Using inverse prediction, log₁₀ concentrations were obtained that correspond to 50% Inhibition (log₁₀IC50*) and the log₁₀ persistence index was calculated for each compound and run using the following formula: log₁₀ persistence Index=log₁₀IC50*₄₈ hrs−log₁₀IC50*_{0 hrs}. Next, a linear mixed effects model was fit on log₁₀ persistence index values with a fixed effect for compound and a random effect for experimental run, followed by post hoc contrasts to compare the log₁₀ persistence index of the positive control EFdA to the log₁₀ persistence index of other test compounds. The estimated LSMeans and differences were then back-transformed via 10^{Estimate} to the original scale and reported as persistence index and fold change respectively. Raw p-values were reported. Antiviral persistence data for representative examples and EFdA are shown in Table 5.

Advantageously, various prodrugs of EFdA of the invention show significant increases in antiviral persistence relative to EFdA, as evidenced by their substantially reduced persistence indicies (Table 5).

TABLE 5

| | WT IC₅₀ | WT IC₅₀ | | Fold | |
|---|---|---|---|---|---|
| Exam- | (μM) | (μM) | Persistence | Change | p- |
| ple | at t = 0 h | at t = 48 h | Index | vs EFdA | Value |
| EFdA | 0.0074 | 0.3156 | 42.87 | 1.00 | — |
| 1 | 0.0760 | 2.2020 | 37.4 | 1.15 | 0.8610 |
| 2 | 0.0020 | 0.0360 | 2.75 | 2.33 | 0.1839 |
| 6 | 0.4103 | 2.1847 | 4.7 | 9.12 | 0.0020 |
| 7 | 0.0051 | 0.1271 | 21.4 | 2.00 | 0.3129 |
| 14 | 0.0064 | 0.0729 | 11.4 | 3.76 | 0.0401 |

### Rat Pharmacokinetics Data for Compounds 6 and 21 (Examples 6 and 21)

### Rat Pharmacokinetics for Subcutaneous Dosing of Compound 6

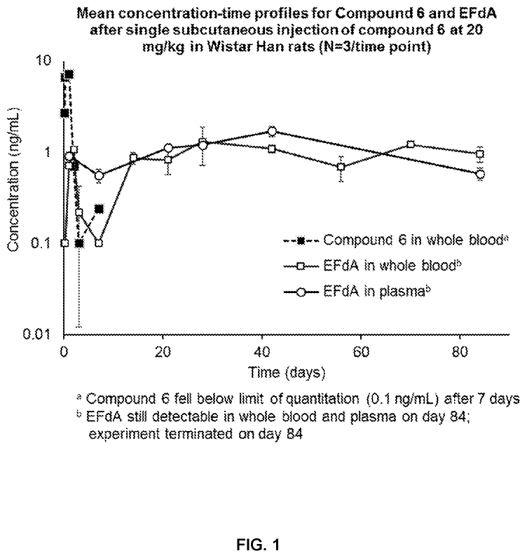

The pharmacokinetics of Compound 6 after a single subcutaneous (SC) injection were evaluated in Male Wistar Han rats. Compound 6 was suspended in a 2% P407, 2% PEG3350, 3.5% Mannitol formulation at 50 mg/mL concentration and sterilized via gamma irradiation. A single dose of 20 mg/kg of Compound 6 was injected (0.4 mL/kg) SC into the lower scapular region (n=3). Blood samples were collected via a lateral tail vein or tail tip amputation at the following time points: Day 0 [120, 360 min], Day 1 [1440 min], Day 2 [2880 min], Days 7, 14, 21, 28, 42, 56, 70, 84. To evaluate Compound 6 and EFdA concentrations, approximately 50 uL of blood was collected into a NaFI/Na2EDTA tube. Exactly 50 uL of blood was then pipetted into a new tube, mixed with 50 uL of 100 mM ammonium acetate pH4, vortexed, immediately frozen on dry ice and stored at −80° C. until analysis. To assess plasma concentrations of EFdA, approximately 200 uL of whole blood was collected into a K2 EDTA tube on days 1, 7, 21, 28, 42, and 84 and stored at 4° C. until analysis. Plasma was separated via centrifugation under standard conditions, frozen on dry ice and stored at −80° C. until analysis. For analysis of Compound 6 and EFdA in whole blood, frozen blood samples were thawed and 30 uL aliquots transferred to separate wells of a 96-well plate. Following addition of 30 uL of diluent, all samples were treated with 200 uL of internal standard (200 ng/mL Warfarin) solution in acetonitrile. The plate was vortexed vigorously for 10 min and then centrifuged for 10 min at 4000 rpm at 15° C. Following centrifugation, 100 uL aliquots of supernatant were transferred to a new 96-well plate containing 100 uL of water in each well. The plate was vortexed for approximately 10 min, and then aliquotes transferred for LC-MS/MS analysis. For analysis of Compound 6 and EFdA in plasma, frozen plasma samples were thawed and 20 uL aliquots transferred to separate wells of a 96-well plate. Following addition of 20 uL of diluent, all samples were treated with 200 uL of internal standard (200 ng/mL Warfarin) solution in acetonitrile. The plate was vortexed vigorously for 10 min and then centrifuged for 10 min at 4000 rpm at 15° C. Following centrifugation, 100 uL aliquots of supernatant were transferred to a new 96-well plate containing 100 uL of water in each well. The plate was vortexed for approximately 10 min, and then aliquotes transferred for LC-MS/MS analysis. LC-MS/MS analysis was performed on the following system: Shimadzu Nexera LC-30AD HPLC pump, Shimadzu Nexera X2 SIL 30ACMP autosampler, SCIEX QTRAP 5500 LC/MS/MS system. Pharmacokinetic parameters were evaluated using Phoenix WinNonLin. The associated concentration-time curves, shown in FIG. 1, demonstrate sustained exposure to EFdA out to 84 days.

### Rat Pharmacokinetics for Intramuscular Dosing of Compounds 6 and 21

The pharmacokinetics of Compounds 6 and 21 after a single intramuscular (IM) injection were evaluated in Male Wistar Han rats. The test compound was suspended in a 2% P407, 2% PEG3350, 3.5% Mannitol formulation at 10 mg/mL concentration. A single dose of 20 mg/kg of test compound was injected (2 mL/kg) IM into the right gastrocnemius muscle (n=3). Blood samples were collected via a lateral tail vein at the following time points: Day 1 [30 min, 1 h, 3 h, 5 h, 7 h], Days 2-5, Days 7, 10, 14, 17, 21, 24, 28, 31, 35, 38, 42, 45, 49, 52, 56, 59, 63, 66 and 70, etc. To evaluate test compound and EFdA concentrations, approximately 150 μL of blood was collected into a NaFL/Na2EDTA tube. Exactly 150 μL of blood was then pipetted into a new tube, mixed with 150 μL of 100 mM ammonium acetate pH4, (some of the prodrugs required mixing with 1.5 μL of FA as stabilizer), vortexed, immediately frozen on dry ice and stored at −80° C. until analysis. For analysis of test compound and EFdA, frozen blood samples were thawed and mixed with 200 μL of internal standard solution (20 ng/mL Glipizide in acetonitrile), vortexed for 10 min at 750 rpm and centrifuged at 6000 rpm for 10 min. The supernatants were then analyzed by UPLC/MS-MS (Triple Quad™ 6500+). Pharmacokinetic parameters were evaluated using a non-compartmental model of the non-compartmental analysis tool, Pharsight Phoenix WinNonlin® 6.4 software.

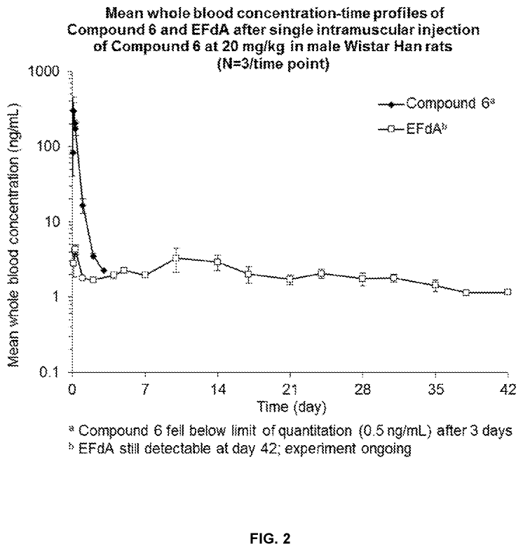

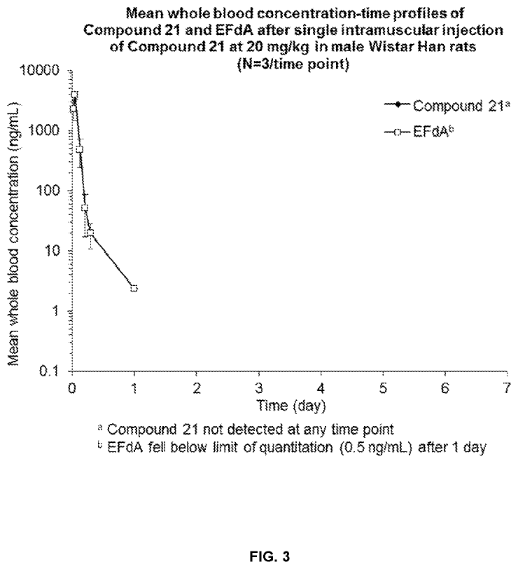

The associated concentration-time curve for Compound 6, shown in FIG. 2, demonstrates sustained exposure to EFdA out to 42 days, which is highly advantageous and unexpected. In contrast, Compound 21 does not afford a sustained release profile, with EFdA levels falling below the limit of quantitation after 1 day (FIG. 3).

1-31. (canceled)

32. A compound of the formula: 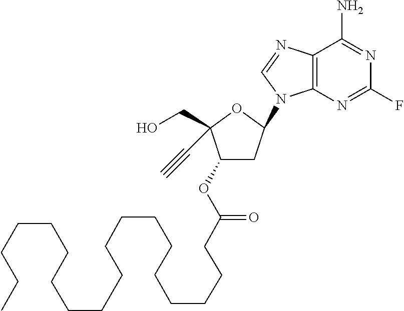
or a pharmaceutically acceptable salt thereof.

33. A pharmaceutical composition comprising a compound according to claim 32, or a pharmaceutically acceptable salt thereof, and a pharmaceutically acceptable excipient.

34. The composition of claim 32, wherein the composition is present in parenteral form.

35. The composition of claim 32, wherein the composition is in a tablet form.

36. A method of treating an HIV infection in a subject comprising administering to the subject a compound of claim 32, or a pharmaceutically acceptable salt thereof.

37. A method of preventing an HIV infection in a subject at risk for developing an HIV infection, comprising administering to the subject a compound of claim 32, or a pharmaceutically acceptable salt thereof.

38. A compound of the formula:

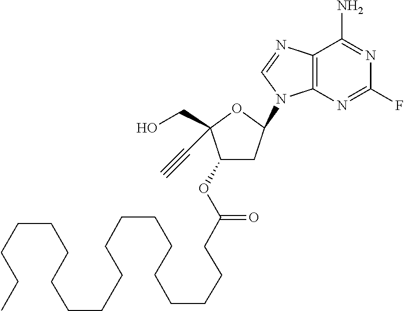

39. A pharmaceutical composition comprising a compound according to claim 38, and a pharmaceutically acceptable excipient.

40. The composition of claim 38, wherein the composition is present in parenteral form.

41. The composition of claim 38, wherein the composition is in a tablet form.

42. A method of treating an HIV infection in a subject comprising administering to the subject a compound of claim 38.

43. A method of preventing an HIV infection in a subject at risk for developing an HIV infection, comprising administering to the subject a compound of claim 38.

44. A combination comprising 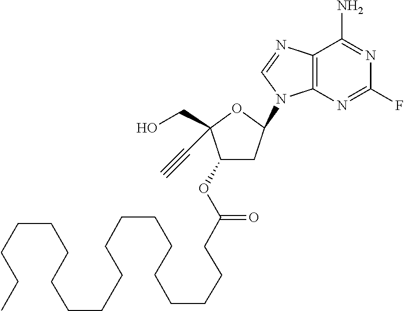
or a pharmaceutically acceptable salt thereof and one or more agents useful against HIV.

45. The combination according to claim 44, wherein the combination is a fixed dose combination.

46. A pharmaceutical composition comprising a combination according to claim 44, and a pharmaceutically acceptable excipient.

47. The composition of claim 44, wherein the composition is present in parenteral form.

48. The composition of claim 44, wherein the composition is in a tablet form.

49. A method of treating an HIV infection in a subject comprising administering to the subject a combination of claim 44.

50. A method of preventing an HIV infection in a subject at risk for developing an HIV infection, comprising administering to the subject a combination of claim 44.
